(19) United States
(12) Patent Application Publication
(10) Pub. No.: US 2023/0063321 A1
(43) Pub. Date: Mar. 2, 2023

(54) Methods for Treating Inflammatory Bowel Diseases with alpha4beta7 Integrin Antagonists

(71) Applicants: Suneel Kumar GUPTA, Sunnyvale, CA (US); Nishit Bachulal MODI, Sunnyvale, CA (US); Protagonist Therapeutics, Inc., Newark, CA (US)

(72) Inventors: Suneel Kumar Gupta, Sunnyvale, CA (US); Nishit Bachulal Modi, Sunnyvale, CA (US); Xiaoli Cheng, Mountain View, CA (US); David Y. Liu, Palo Alto, CA (US); Larry C. Mattheakis, Cupertino, CA (US)

(21) Appl. No.: 17/788,179

(22) PCT Filed: Jan. 8, 2021
(86) PCT No.: PCT/US2021/012842

§ 371 Date: Jun. 22, 2022

Related U.S. Application Data
(60) Provisional application No. 62/959,854, filed on Jan. 10, 2020.

(51) Int. Cl. A61K 47/64; A61K 9/00; A61P 1/04

(52) U.S. Cl. CPC A61K 47/64; A61K 9/0053; A61P 1/04

(57) ABSTRACT

The invention relates to methods of treating inflammatory bowel diseases, including with engineered peptides (e.g. peptide monomers and dimers comprising disulfide or thioether intramolecular bonds) that bind α4β7 integrin. In one aspect, the disclosure provides a method of treating an inflammatory bowel disease (IBD) in a subject in need thereof, comprising administering to the subject an α4β7 integrin antagonist, wherein the antagonist is administered to the patient orally at a dose of about 100 mg to about 500 mg, once or twice daily, wherein the antagonist is a peptide dimer compound comprising two peptides, or a pharmaceutically acceptable salt thereof.

### CROSS REFERENCE TO RELATED APPLICATIONS

This application claims priority to U.S. Provisional Application No. 62/959,854, filed on Jan. 10, 2020; which is herein incorporated by reference in its entirety.

### SEQUENCE LISTING

This application is being filed electronically via EFS-Web and includes an electronically submitted sequence listing in .txt format. The .txt file contains a sequence listing entitled “PRTH_052_01WO_ST25.txt” created on Jan. 8, 2021 and having a size of ˜7 kilobytes. The sequence listing contained in this .txt file is part of the specification and is incorporated herein by reference in its entirety.

### FIELD OF THE INVENTION

The present disclosure relates to methods of treating inflammatory bowel diseases with engineered peptides (e.g. peptide monomers and dimers comprising disulfide or thioether intramolecular bonds) that bind α4β7 integrin.

### BACKGROUND OF THE INVENTION

Integrins are noncovalently associated a/P heterodimeric cell surface receptors involved in numerous cellular processes ranging from cell adhesion and migration to gene regulation (Dubree, et al., Selective α4β7 Integrin Antagonist and Their Potential as Anti-inflammatory Agents, J. Med. Chem. 2002, 45, 3451-3457). Differential expression of integrins can regulate a cell's adhesive properties, allowing different leukocyte populations to be recruited to specific organs in response to different inflammatory signals. If left unchecked, the integrin-mediated adhesion process can lead to chronic inflammation and autoimmune disease.

The α4 integrins, α4β1 and α4β7, play essential roles in lymphocyte migration throughout the gastrointestinal tract. They are expressed on most leukocytes, including B and T lymphocytes, where they mediate cell adhesion via binding to their respective primary ligands, vascular cell adhesion molecule (VCAM), and mucosal addressin cell adhesion molecule 1 (MAdCAMI1), respectively. The proteins differ in binding specificity in that VCAM binds both α4β1 and to a lesser extent α4β7, while MAdCAM1 is highly specific for α4β7. In addition to pairing with the α4 subunit, the β7 subunit also forms a heterodimeric complex with αE subunit to form α4β7, which is primarily expressed on intraepithelial lymphocytes (IEL) in the intestine, lung and genitourinary tract. α4β7 is also expressed on dendritic cells in the gut. The α4β7 heterodimer binds to E-cadherin on the epithelial cells. The IEL cells are thought to provide a mechanism for immune surveillance within the epithelial compartment. Therefore, blocking α4β7 and α4β7 together may be a useful method for treating inflammatory conditions of the intestine.

Inhibitors of specific integrins-ligand interactions have been shown effective as anti-inflammatory agents for the treatment of various autoimmune diseases. For example, monoclonal antibodies displaying high binding affinity for α4β7 have displayed therapeutic benefits for gastrointestinal auto-inflammatory/autoimmune diseases, such as Crohn's disease, and ulcerative colitis (Id). However, these therapies interfered with α4β1 integrin-ligand interactions thereby resulting in dangerous side effects to the patient. Therapies utilizing small molecule antagonists have shown similar side effects in animal models, thereby preventing further development of these techniques. More recently engineered peptides showing high potency and stability, as well as high specifity for α4β7 integrins, have been shown to be effective in the treatment of various immune disorders, including inflammatory bowel disease.

However, there is a need in the art for additional methods of using α4β7 antagonists and other agents for treating inflammatory disorders. Such methods are disclosed herein.

### SUMMARY OF THE INVENTION

The present disclosure provides composition and methods for treating various diseases and conditions associated with α4β7 integrin signaling.

In one aspect, the disclosure provides a method of treating an inflammatory bowel disease (IBD) in a subject in need thereof, comprising administering to the subject an α4β7 integrin antagonist, wherein the antagonist is administered to the patient orally at a dose of about 100 mg to about 500 mg, once or twice daily, wherein the antagonist is a peptide dimer compound comprising two peptides, or a pharmaceutically acceptable salt thereof, wherein each of the two peptides comprises or consists of any of the sequences (optionally with an N-terminal Ac):

(SEQ ID NO: 1)
2-methylbenzoyl-(N-Me-Arg)-Ser-Asp-Thr-Leu-Pen-
Phe(4-tBu)-(3-homo-Glu)-(D-Glu)-(D-Lys)-OH;
(SEQ ID NO: 2)
2-methylbenzoyl-(N-Me-Arg)-Ser-Asp-Thr-Leu-Pen-
Phe(4-tBu)-(P-homo-Glu)-Gly-(D-Lys)-OH;
(SEQ ID NO: 3)
2-methylbenzoyl-(N-Me-Arg)-Ser-Asp-Thr-Leu-Pen-
Phe(4-tBu)-(P-homo-Glu)-Pro-(D-Lys)-OH;
(SEQ ID NO: 4)
2-methylbenzoyl-(N-Me-Arg)-Ser-Asp-Thr-Leu-Pen-
Phe(4-tBu)-(P-homo-Glu)-(D-Pro)-(D-Lys)-OH;
(SEQ ID NO: 5)
2-methylbenzoyl-(N-Me-Arg)-Ser-Asp-Thr-Leu-Pen-
Phe(4-tBu)-(P-homo-Glu)-(D-Lys)-OH;
(SEQ ID NO: 5)
2-methylbenzoyl-(N-Me-Arg)-Ser-Asp-Thr-Leu-Pen-
Phe(4-tBu)-(P-homo-Glu)-(D-Lys)-NH₂;
(SEQ ID NO: 6)
Pen-(N-Me-Arg)-Ser-Asp-Thr-Leu-Pen-Phe(4-tBu)-
(P-homo-Glu)-(D-Lys)-OH;
(SEQ ID NO: 6)
Pen-(N-Me-Arg)-Ser-Asp-Thr-Leu-Pen-Phe(4-tBu)-
(P-homo-Glu)-(D-Lys)-NH₂;
(SEQ ID NO: 7)
Pen-(N-Me-Arg)-Ser-Asp-Thr-Leu-Pen-Phe(4-tBu)-
(P-homo-Glu)-Pro-(D-Lys)-OH;
(SEQ ID NO: 7)
Pen-(N-Me-Arg)-Ser-Asp-Thr-Leu-Pen-Phe(4-tBu)-
(P-homo-Glu)-Pro-(D-Lys)-NH₂;
(SEQ ID NO: 8)
Pen-(N-Me-Arg)-Ser-Asp-Thr-Leu-Pen-Phe(4-tBu)-
(P-homo-Glu)-(D-Pro)-(D-Lys)-OH;
or
(SEQ ID NO: 8)
Pen-(N-Me-Arg)-Ser-Asp-Thr-Leu-Pen-Phe(4-tBu)-
(P-homo-Glu)-(D-Pro)-(D-Lys)-NH₂;

wherein each of the two peptides comprises a thioether bond between the 2-methylbenzoyl and the Pen or a disulfide bond between the two Pens, wherein the two peptides are linked by a linker moiety bound to the D-Lys amino acids of the two peptides, and wherein the linker moiety is diglycolic acid (DIG).

In one embodiment, each of the two peptides consists of the sequence:

(SEQ ID NO: 1)
2-methylbenzoyl-(N-Me-Arg)-Ser-Asp-Thr-Leu-Pen-
Phe(4-tBu)-(3-homo-Glu)-(D-Glu)-(D-Lys)-OH,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In one embodiment, each of the two peptides consists of the sequence:

(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In one embodiment, each of the two peptides consists of the sequence:

(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In one embodiment, each of the two peptides consists of the sequence:

(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In one embodiment, each of the two peptides consists of the sequence:

(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In one embodiment, each of the two peptides consists of the sequence:

(SEQ ID NO: 5)
2-methylbenzoyl-(N-Me-Arg)-Ser-Asp-Thr-Leu-Pen-
Phe(4-tBu)-(β-homo-Glu)-(D-Lys)-OH,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In one embodiment, the peptide dimer compound or pharmaceutically acceptable salt thereof is:

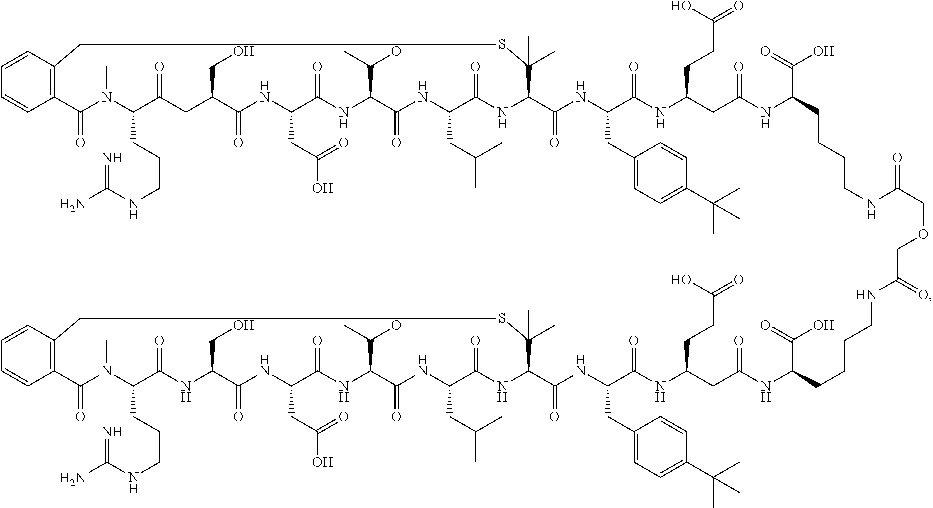 [SMILES: CC(C)C[C@@H]1NC(=O)[C@H](C(C)O)NC(=O)[C@H](CC(=O)O)NC(=O)[C@H](CO)CC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

or a pharmaceutically acceptable salt thereof.

In one embodiment, the peptide dimer compound or pharmaceutically acceptable salt thereof is:

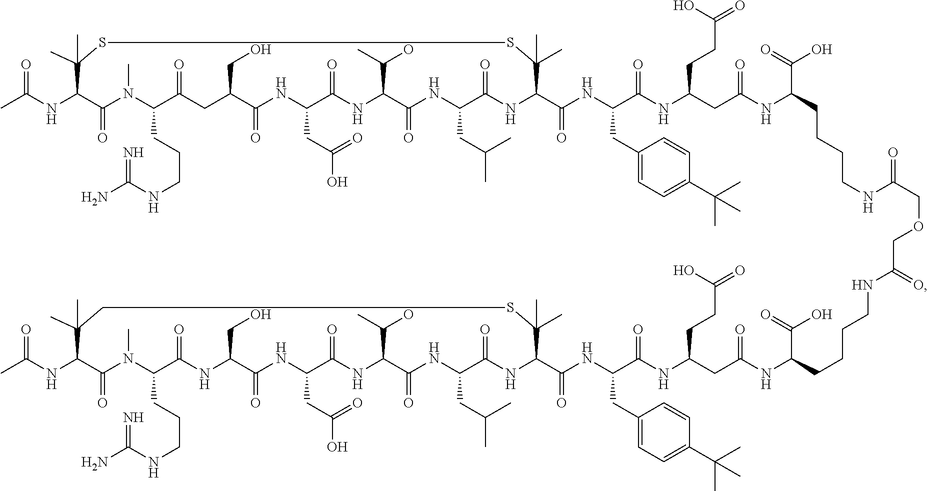 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)CC(=O)[C@H](CCCNC(=N)N)N(C)C(=O)[C@@H](NC(C)=O)C(C)(C)SSC2(C)C)C(=O)O)C(=O)O)C(C)(C)SCC1(C)C]

or a pharmaceutically acceptable salt thereof.

In one embodiment, each of the two peptides comprises or consists of the sequence:

(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In one embodiment, each of the two peptides comprises or consists of the sequence:

(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In one embodiment, each of the two peptides comprises or consists of the sequence:

(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In one embodiment, each of the two peptides comprises or consists of the sequence:

(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In one embodiment, each of the two peptides comprises or consists of the sequence:

(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In one embodiment, each of the two peptides comprises or consists of the sequence:

(SEQ ID NO: 5)
2-methylbenzoyl-(N-Me-Arg)-Ser-Asp-Thr-Leu-Pen-
Phe(4-tBu)-(β-homo-Glu)-(D-Lys)-OH,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).
Any of the peptides disclosed herein may include an N-terminal Ac.

In one embodiment, the peptide dimer compound or pharmaceutically acceptable salt thereof is:

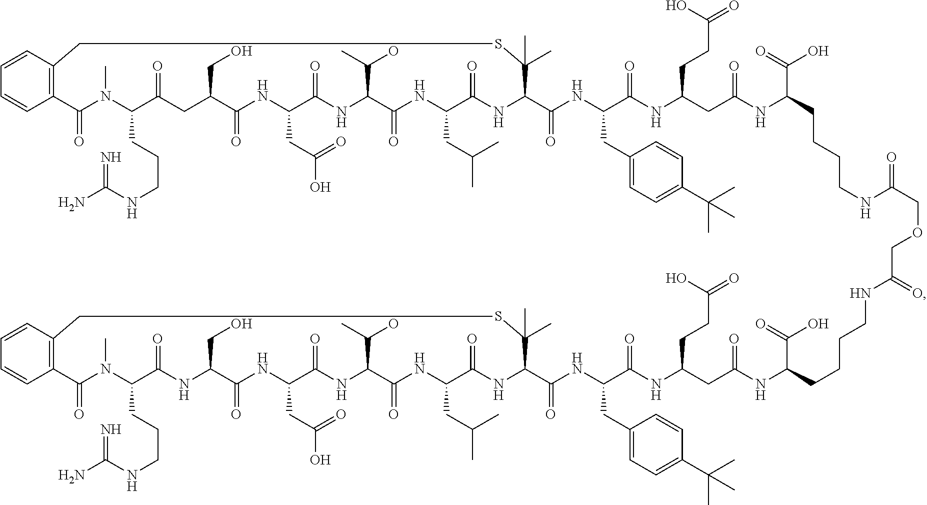 [SMILES: CC(C)C[C@@H]1NC(=O)[C@H](C(C)O)NC(=O)[C@H](CC(=O)O)NC(=O)[C@H](CO)CC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

or a pharmaceutically acceptable salt thereof.

In one embodiment, the peptide dimer compound or pharmaceutically acceptable salt thereof is:

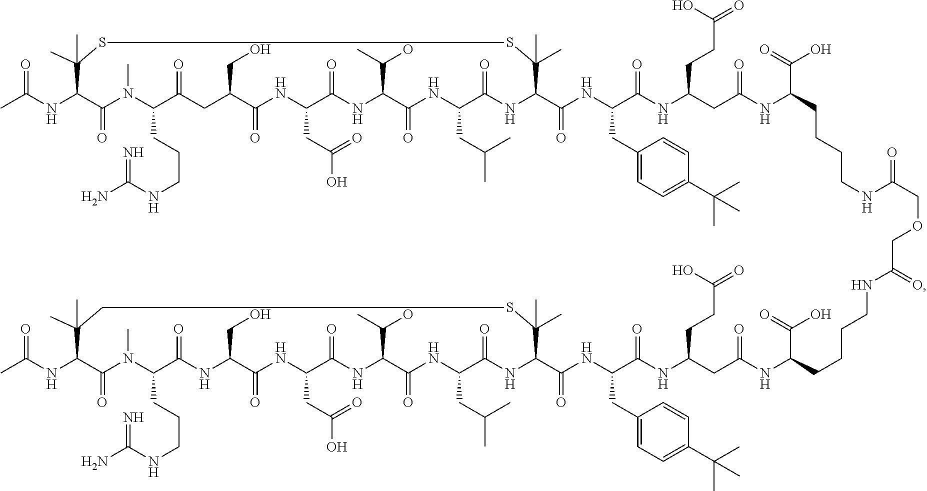 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)CC(=O)[C@H](CCCNC(=N)N)N(C)C(=O)[C@@H](NC(C)=O)C(C)(C)SSC2(C)C)C(=O)O)C(=O)O)C(C)(C)SCC1(C)C]

or a pharmaceutically acceptable salt thereof.

In particular embodiments of the methods disclosed herein, the peptide dimer compound or pharmaceutically acceptable salt thereof is administered to the subject at a dose of about 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, or 500.0 mg. In one embodiment, the peptide dimer compound or pharmaceutically acceptable salt thereof is administered to the subject at a dose of about 150 mg or about 450 mg. In certain embodiments, the dose is administered to the subject twice daily.

In particular embodiments, the pharmaceutically acceptable salt of the peptide dimer compound is an acetate salt.

In particular embodiments of the methods disclosed herein, the dosage administered results in a non-saturating blood receptor occupancy (% RO), optionally when measured at peak blood or serum levels of the antagonist. In some embodiments, the dosage administered results in less than 90% RO, less than 80% RO, less than 70% RO, less than 60% RO, or less than 50% RO, optionally when measured at peak blood or serum levels of the antagonist.

In particular embodiments of the methods disclosed herein, the method inhibits MadCAM1-mediated T cell proliferation in the gastrointestinal tract.

In particular embodiments of the methods disclosed herein, the method reduces cell surface expression of β7 on CD4+ T cells in the gastrointestinal tract.

In particular embodiments of the methods disclosed herein, the method:
i) induces internalization of α4β7 integrin on CD4+ T memory cells;
ii) causes reduced adhesion of CD4+ T memory cells to MAdCAM1 in the gastrointestinal tract; and/or
iii) inhibits homing of T cells to the gastrointestinal tract, optionally to the ileal lamina propia, Peyer's Patches, mesenteric lymph nodes, small intestine, and/or colon.

In particular embodiments of the methods disclosed herein, the IBD is ulcerative colitis.

In particular embodiments of the methods disclosed herein, the IBD is Crohn's disease.

In particular embodiments of the methods disclosed herein, the method results in one or more of the following pharmacokinetic parameters in plasma of the subject.
Cmax (ng/mL) of 1-25;
Tmax (h) of 1-5;
AUCₜ (ng·h/mL) of 10-250
AUC_{inf} (ng·h/mL) of 10-300;
t_{1/2} (h) of 3-10;
AUCₜₐᵤ (ng·h/mL) of 30-130;
Ctrough (ng/mL) of 1-5;
accumulation Cmax (ng·mL) of 0.5-2.5; and
accumulation AUCₜ (ng·h/mL) of 0.5-3.0.

In particular embodiments of the methods disclosed herein, the method results in one or more of the following pharmacodynamic parameters in plasma of the subject.
ROmax (%) of 50-100;
change in receptor expressionₘₐₓ (%) of −20 to −60;
average change in receptor expression (%) of −10 to −55;
steady state ROmax (%) of 80-100;
average RO₀₋₂₄ (% h) of 50-95;
average RO₀₋₁₂ (% h) of 80-95; and
average RO₁₂₋₂₄ (% h) of 70-90.

In another aspect, the disclosure provides a method of treating an inflammatory disease or disorder in a subject in need thereof, comprising administering to the subject an α4β7 integrin antagonist, wherein the antagonist is administered at a dosage that results in a non-saturating blood receptor occupancy (% RO), optionally when measured at peak blood or serum levels of the antagonist. In certain embodiments, the antagonist is administered at a dosage that results in less than 90% blood RO, less than 80% blood RO, less than 70% blood RO, less than 60% blood RO, or less than 50% blood RO, optionally when measured at peak blood or serum levels of the antagonist. In certain embodiments, the antagonist is present in a pharmaceutical composition formulated for a route of administration selected from oral administration, parenteral administration, subcutaneous administration, buccal administration, nasal administration, administration by inhalation, topical administration, and rectal administration. In certain embodiments, the antagonist is administered orally or rectally.

In certain embodiments of any of the methods disclosed, the inflammatory disease or disorder is selected from the group consisting of: Inflammatory Bowel Disease (IBD), adult IBD, pediatric IBD, adolescent IBD, ulcerative colitis, Crohn's disease, Celiac disease (nontropical Sprue), enteropathy associated with seronegative arthropathies, microscopic colitis, collagenous colitis, eosinophilic gastroenteritis, radiotherapy, chemotherapy, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, primary sclerosing cholangitis, human immunodeficiency virus (HIV) infection in the GI tract, eosinophilic asthma, eosinophilic esophagitis, gastritis, colitis, microscopic colitis, and graft versus host disease (GVDH). In particular embodiments, the disease or disorder is an IBD, such as ulcerative colitis or Crohn's disease.

In certain embodiments, the antagonist is a peptide dimer compound comprising two peptides, or a pharmaceutically acceptable salt thereof,

wherein each of the two peptides comprises or consists of any of the sequences (optionally including an N-terminal Ac):

(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH;
(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;
(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;
(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;
(SEQ ID NO: 5)
2-methylbenzoyl-(N-Me-Arg)-Ser-Asp-Thr-Leu-Pen-
Phe(4-tBu)-(β-homo-Glu)-(D-Lys)-OH;
(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂;
(SEQ ID NO: 6)
Pen-(N-Me-Arg)-Ser-Asp-Thr-Leu-Pen-Phe(4-tBu)-
(β-homo-Glu)-(D-Lys)-OH;
(SEQ ID NO: 6)
Pen-(N-Me-Arg)-Ser-Asp-Thr-Leu-Pen-Phe(4-tBu)-
(β-homo-Glu)-(D-Lys)-NH2;
(SEQ ID NO: 7)
Pen-(N-Me-Arg)-Ser-Asp-Thr-Leu-Pen-Phe(4-tBu)-
(β-homo-Glu)-Pro-(D-Lys)-OH;
(SEQ ID NO: 7)
Pen-(N-Me-Arg)-Ser-Asp-Thr-Leu-Pen-Phe(4-tBu)-
(β-homo-Glu)-Pro-(D-Lys)-NH2;
(SEQ ID NO: 8)
Pen-(N-Me-Arg)-Ser-Asp-Thr-Leu-Pen-Phe(4-tBu)-
(β-homo-Glu)-(D-Pro)-(D-Lys)-OH;
or
(SEQ ID NO: 8)
Pen-(N-Me-Arg)-Ser-Asp-Thr-Leu-Pen-Phe(4-tBu)-
(β-homo-Glu)-(D-Pro)-(D-Lys)-NH2;

wherein each of the two peptides comprises a thioether bond between the 2-methylbenzoyl and the Pen; or a disulfide between the two Pens; wherein the two peptides are linked by a linker moiety bound to the D-Lys amino acids of the two peptides, and wherein the linker moiety is diglycolic acid (DIG).

In particular embodiments, each of the two peptides comprises or consists of the sequence:

(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, each of the two peptides comprises or consists of the sequence:

(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, each of the two peptides comprises or consists of the sequence:

(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, each of the two peptides comprises or consists of the sequence:

(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, each of the two peptides comprises or consists of the sequence:

2-methylbenzoyl-(N-Me-Arg)-Ser-Asp-Thr-Leu-Pen-Phe(4-tBu)-(D-homo-Glu)-(D-Lys)-NH2 (SEQ ID NO:5),
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, each of the two peptides comprises or consists of the sequence:

2-methylbenzoyl-(N-Me-Arg)-Ser-Asp-Thr-Leu-Pen-Phe(4-tBu)-(D-homo-Glu)-(D-Lys)-OH (SEQ ID NO:5),
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, the peptide dimer compound or pharmaceutically acceptable salt thereof is:

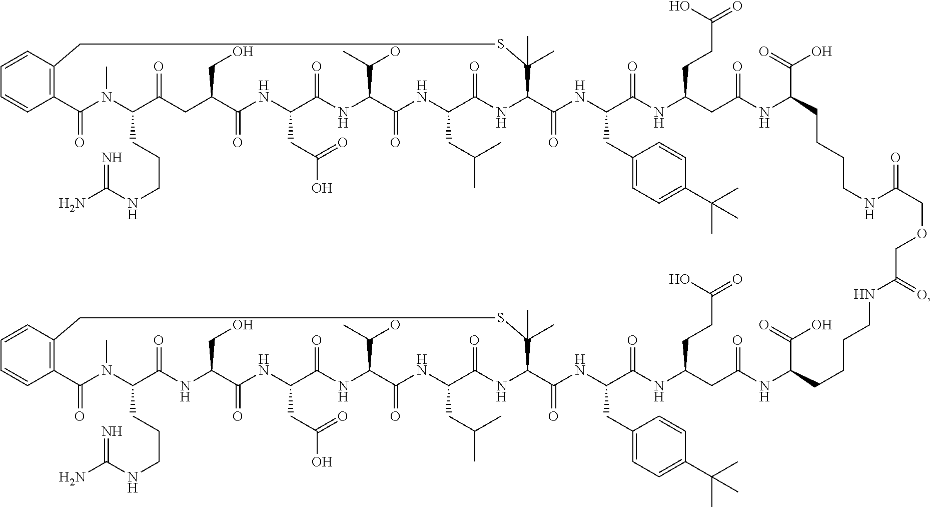 [SMILES: CC(C)C[C@@H]1NC(=O)[C@H](C(C)O)NC(=O)[C@H](CC(=O)O)NC(=O)[C@H](CO)CC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

or a pharmaceutically acceptable salt thereof.

In particular embodiments, the peptide dimer compound or pharmaceutically acceptable salt thereof is:

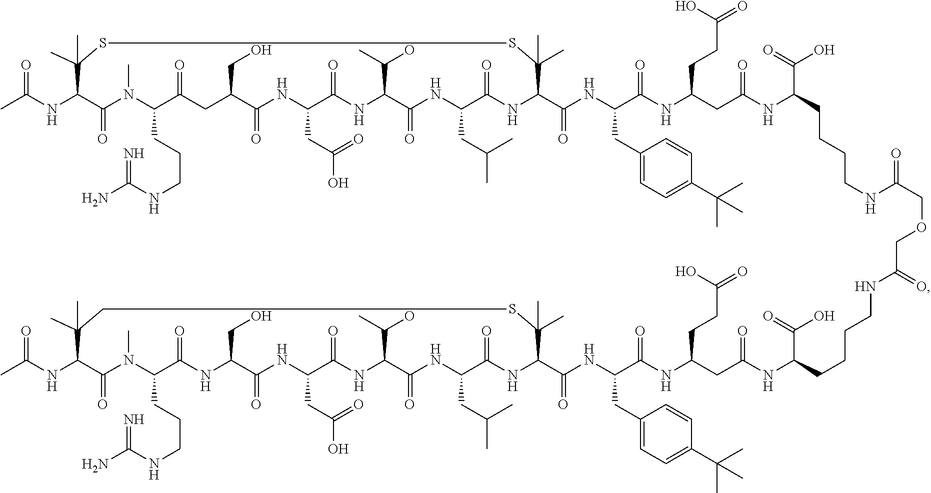 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)CC(=O)[C@H](CCCNC(=N)N)N(C)C(=O)[C@@H](NC(C)=O)C(C)(C)SSC2(C)C)C(=O)O)C(=O)O)C(C)(C)SCC1(C)C]

or a pharmaceutically acceptable salt thereof.

In particular embodiments, each of the two peptides comprises or consists of the sequence:

(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, each of the two peptides comprises or consists of the sequence:

(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, each of the two peptides comprises or consists of the sequence:

(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, each of the two peptides comprises or consists of the sequence:

(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, each of the two peptides comprises or consists of the sequence:

(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, each of the two peptides consists of the sequence:

(SEQ ID NO: 5)
2-methylbenzoyl-(N-Me-Arg)-Ser-Asp-Thr-Leu-Pen-
Phe(4-tBu)-(β-homo-Glu)-(D-Lys)-OH,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In particular embodiments, the peptide dimer compound or pharmaceutically acceptable salt thereof is:

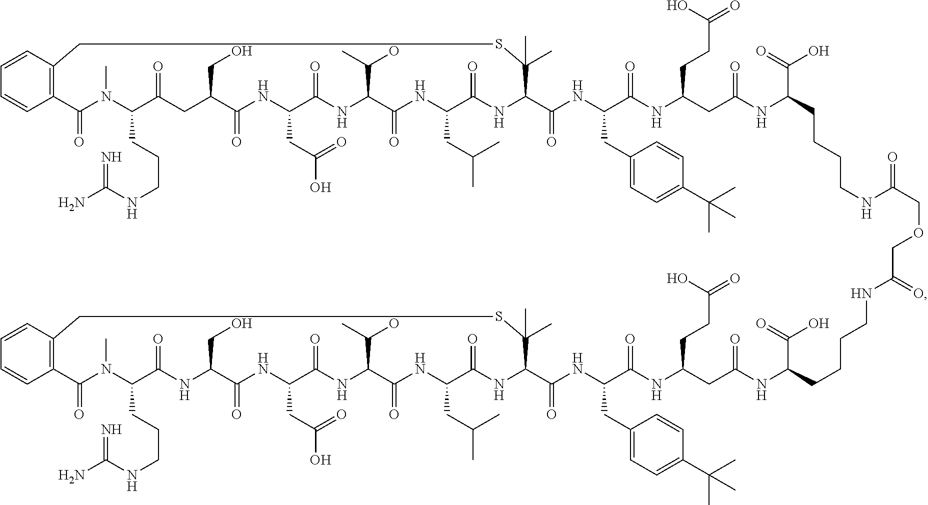 [SMILES: CC(C)C[C@@H]1NC(=O)[C@H](C(C)O)NC(=O)[C@H](CC(=O)O)NC(=O)[C@H](CO)CC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

or a pharmaceutically acceptable salt thereof.

In particular embodiments, the peptide dimer compound or pharmaceutically acceptable salt thereof is:

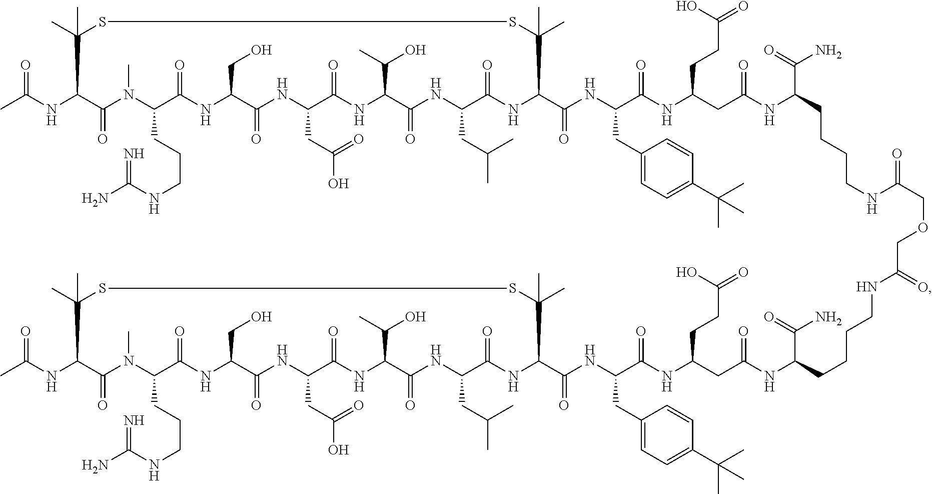 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)[C@@H](NC(C)=O)C(C)(C)SSC2(C)C)C(N)=O)C(N)=O)C(C)(C)SSC1(C)C]

or a pharmaceutically acceptable salt thereof.

In particular embodiments, the peptide dimer compound or pharmaceutically acceptable salt thereof is administered to the subject at a dose of about 5, 6, 7, 8, 9, 10, 12.5, 25.0, 37.5, 50.0, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, or 500.0 mg. In particular embodiments, the dose is administered to the subject once a day or twice a day.

In particular embodiments, the pharmaceutically acceptable salt of the peptide dimer compound is an acetate salt.

In a related aspect, the disclosure provides a pharmaceutical composition comprising a peptide dimer compound or pharmaceutically acceptable salt thereof disclosed in any one of claims 39-58. In particular embodiments, the composition is formulated for oral delivery, optionally wherein the composition comprises an enteric coating. In particular embodiments, the method comprises administering to the subject the pharmaceutical composition disclosed herein.

In particular embodiments of methods and compositions disclosed herein, the antagonist or pharmaceutically acceptable salt thereof inhibits binding of α4β7 integrin to MAdCAM1.

In particular embodiments of methods and compositions disclosed herein, the antagonist or pharmaceutically acceptable salt thereof or the pharmaceutical composition is provided to the subject in need thereof at an interval sufficient to improve or ameliorate the condition. In particular embodiments, the interval is selected from the group consisting of: around the clock, hourly, every four hours, once daily, twice daily, three times daily, four times daily, every other day, weekly, bi-weekly, and monthly. In certain embodiments, the antagonist or pharmaceutically acceptable salt thereof or pharmaceutical composition is provided as an initial does followed by one or more subsequent doses, and the minimum interval between any two doses is a period of less than 1 day, and wherein each of the doses comprises an effective amount of the antagonist. In some embodiments, the effective amount of the antagonist or pharmaceutically acceptable salt thereof or the pharmaceutical composition is sufficient to achieve at least one of the following.

a) about 50% or greater saturation of MAdCAM1 binding sites on α4β7 integrin molecules;
b) about 50% or greater inhibition of α4β7 integrin expression on the cell surface; and
c) about 50% or greater saturation of MAdCAM1 binding sites on α4β7 molecules and about 50% or greater inhibition of α4β7 integrin expression on the cell surface, wherein i) the saturation is maintained for a period consistent with a dosing frequency of no more than twice daily; ii) the inhibition is maintained for a period consistent with a dosing frequency of no more than twice daily; or iii) the saturation and the inhibition are each maintained for a period consistent with a dosing frequency of no more than twice daily.

### BRIEF DESCRIPTION OF THE DRAWINGS

FIG. 1. is a table showing proliferation of T cells in response to the indicated treatments and the inhibition of T cell proliferation by Compound A or Vedolizumab.

FIG. 2 is a table showing CD45RO⁻ naïve and CD45RO⁺ memory T-cells in response to treatment with ati-CD3 or anti-CD3+MAdaCAM.

FIGS. 3A-sB provides tables showing increased β7 expression upon successive cycles of proliferation (FIG. 3A) and reduced surface expression of β7 in undivided CD4⁺ T-cells in the presence of Compound A (rFIG. 3B).

FIG. 4 is a graph showing the reduction in surface β7 expression in five donors upon treatment with Compound A.

FIGS. 5A-C are graphs showing cytokine release following treatment with anti-CD3+MAdCAM1 and inhibition by Compound A for the following cytokines: IFNγ (FIG. 5A), IL-23 (FIG. 5B), and GM-CSF (FIG. 5C).

FIGS. 6A-C are graphs showing cytokine release following treatment with anti-CD3+MAdCAM1 and inhibition by Compound A for the following cytokines: IL-10 (FIG. 6A), IL-5 (FIG. 6B), and TNFα (FIG. 6C).

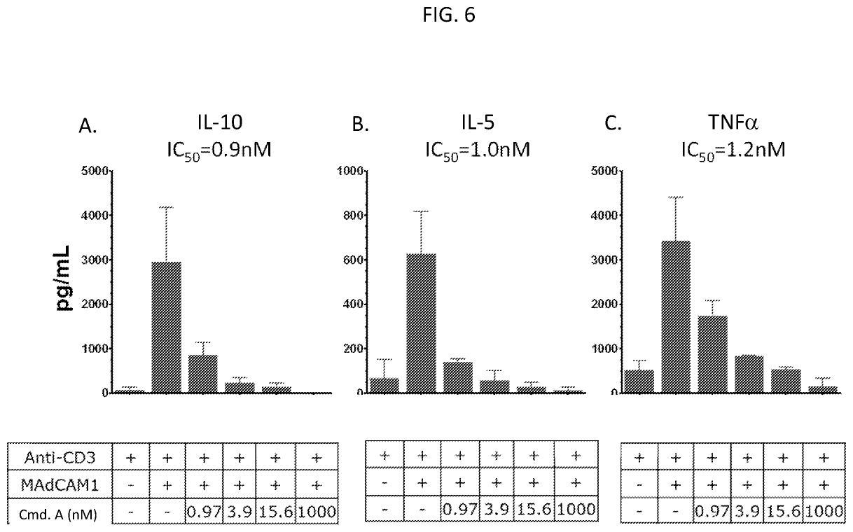

FIG. 7 is a graph showing % receptor occupancy (RO) in whole blood and Peyer's Patches following administration of the indicated doses of Compound A. The table below the graph provides the % RO.

FIG. 8 provides % RO in whole blood and Peyer's Patches for six individual animals treated with the indicated amounts of Compound A (upper panel). % RO on day 14 is provided in the lower panel.

FIG. 9 provides graphs showing the concentration of Compound A in plasma and Peyer's Patches following administration of the indicated doses of Compound A (left panel) and % RO of Compound A in whole blood and Peyer's Patches following administration of the indicated doses of Compound A (right panel).

FIG. 10 provides graphs showing the concentration of Compound A detected in plasma and the indicated tissues at various time points following treatment. The lower panel represents data from the upper panel plotted with an expanded scale.

FIG. 11 is a graph summarizing various pharmacokinetic parameters for Compound A following single PO dose administration at 30 mg/kg in mice.

FIG. 12 is a graph showing the percentage of cultured cells having the indicated surface markers following the indicated treatments. For each cell type, the four bars from left to right correspond to the treatments indicated on the left from top to bottom.

FIG. 13 is a graph showing the percentage of cultured cells having the indicated surface markers following the indicated treatments. For each cell type, the four bars from left to right correspond to the treatments indicated on the left from top to bottom.

FIG. 14 is a graph showing α4β7 cell surface expression on PBMCs treated with Compound C or Compound D. FMO is used as a staining control.

FIG. 15 is a graph showing time-dependent α4β7 cell surface expression on CD4+ T memory cells treated with Compound A at time 0.

FIG. 16 is a graph showing concentration-dependent α4β7 cell surface expression on CD4+ T memory cells treated with the indicated concentration of Compound A.

FIG. 17 is a graph showing concentration-dependent α4β7 cell surface expression on CD4+ T memory cells treated with the indicated concentration of Compound A.

FIG. 18 is a graph showing concentration-dependent reduction in adhesion to MAdCAM1 on CD4+ T memory cells treated with the indicated concentration of Compound A.

FIG. 19 is a graph showing the correlation between % reduction in adhesion to MAdCAM1 and % reduction in α4β7 expression.

FIG. 20 is a graph showing downregulation of α4β7 expression following treatment with Compound A, followed by recovery in α4β7 expression after treatment was terminated.

FIG. 21 is a graph showing mean plasma concentration over time following single doses of the indicated amounts of Compound A.

FIGS. 22A-B are graphs showing receptor occupancy (%)(FIG. 22A) and receptor expression (%)(FIG. 22B) over time following single doses of the indicated amounts of Compound A.

FIGS. 23A-B are graphs showing the mean steady-state plasma concentration of Compound A (FIG. 23A) and receptor occupancy (%)(FIG. 23B) over time following administration of 450 mg Compound A as a liquid solution or immediate release tablet.

FIG. 24 is a graph showing correlation between plasma concentration of Compound A and receptor occupancy (%) following administration of Compound A.

FIG. 25 is a graph showing average receptor occupancy (%) in whole blood and Peyer's Patches following administration of the indicated doses of Compound A. The associated numerical values are provided in the table.

FIG. 26 provides graphs showing dose-dependent concentration of Compound A in plasma (left panel) and Peyer's Patches (right panel) following administration of the indicated doses of Compound A.

FIG. 27 is a table showing receptor occupancy in individual animals following treatment with the indicated dose of Compound A.

### DETAILED DESCRIPTION OF THE INVENTION

Ulcerative colitis is a chronic inflammatory bowel disease (IBD) with a remitting and relapsing course, characterized by bloody diarrhea, abdominal cramps, and fatigue. The pathogenesis is thought to result from inappropriate immune response to gastrointestinal antigens and environmental triggers in genetically susceptible individuals. The highest prevalence is reported in Europe and North America. Ulcerative colitis has a significant negative impact on patient quality of life and presents a high economic burden on health systems.

Inflammatory bowel diseases, such as ulcerative colitis, has been managed with corticosteroids, 5-aminosalicylates, and immunosuppressants, and more recently, with the use of biologics targeted against specific mediators of inflammation. Therapeutic options for the long-term treatment of ulcerative colitis are limited. 5-aminosalicylates such as sulfasalazine, olsalazine, balsalazide and various forms of mesalamine (e.g. Asacol, Pentasa, Lialda, Canasa) are only effective in mild- to moderate disease whereas patients with severe disease may be started on biologics. Several monoclonal antibodies against TNF-α (e.g. infliximab, adalimumab, golimumab, and certolizumab) are now available. Agents targeted against other cytokines involved in the inflammatory response such as ustekinumab against IL-12/IL-23, and tofacitinib, a pan-JAK inhibitor, are now part of the therapeutic options available for inflammatory bowel disease, and several IL-23 and S1P1 inhibitors are also currently under clinical investigation.

In spite of the wide array of therapeutic options, there are still limitations in the treatment of inflammatory bowel diseases and the agents available are not without risk. TNF-α inhibitors are ineffective in approximately ⅕ to ⅓ of the patients and 10-15% of treated patients who show an initial benefit may lose response every year. Cutaneous reactions are also most the most common adverse reactions with anti-TNF treatments. This includes injection site reactions, cutaneous infections, immune-mediated complications such as psoriasis and lupus-like syndrome and rarely skin cancers. Tofacitinib can increase the risk of infection and may increase the risk of thrombosis or thromboembolic events. There is increasing recognition that mitigation of the local inflammatory response may hold promise. Orally administered budesonide and 5-ASAs are effective locally, and various other locally acting agents, including AMT-101, a locally acting oral biologic fusion protein of interleukin 10, and TD-1473, a JAK inhibitor, have shown promise or are undergoing clinical investigation. Local delivery through oral administration may allow higher doses of drug to be delivered to the target site without increasing systemic side effects.

Integrins are heterodimers that function as cell adhesion molecules. The α4 integrins, α4β1 and α4β7, are known to play essential roles in lymphocyte migration throughout the gastrointestinal tract. They are expressed on most leukocytes, including B and T lymphocytes, monocytes, and dendritic cells, where they mediate cell adhesion via binding to their respective primary ligands, namely vascular cell adhesion molecule (VCAM) and mucosal addressin cell adhesion molecule 1 (MAdCAM1). VCAM and MAdCAM1 differ in binding specificity, in that VCAM binds both α4β1 and α4β7, while MAdCAM1 is highly specific for α4β7.

The α4β7 integrin, which is primarily involved in the recruitment of leukocytes to the gastrointestinal (GI) tract, is present on the cell surface of a small population of circulating T and B lymphocytes. Its major ligand, MAdCAM1 is selectively expressed on the endothelium of the intestinal vasculature and is present in increased concentrations in inflamed tissue.

The present disclosure provides methods of treating IDs by inhibiting α4β7 integrin, for example, using a peptide dimer antagonist of α4β7 integrin, including but not limited to any of those disclosed herein. In particular, the disclosure provides oral dosages of α4β7 integrin antagonists effective in treating IBDs, including ulcerative colitis. In addition, the disclosure provides pharmacokinetic and pharmacodynamics parameters of α4β7 integrin antagonists associated with biological activity of the antagonists, such as inhibition of MAdCAM1-mediated T cell proliferation, reduced T cell expression of β7 (and α4β7 integrin), internalization of α4β7 integrin on T cells, reduced homing of T cells into gastrointestinal tract tissue, decreased cytokine release by T cells, reduced adhesion of T cells to MAdCAM1, and reduced gastrointestinal tract inflammation. In particular embodiments the T cells are CD4+ T memory cells.

Furthermore, it was previously believed that the mechanism underlying the use of α4β7 integrin antagonists to treat IBDs involved binding of the antagonist to α4β7 expressed on circulating T cells, which prevents the T cells from binding to MAdCAM1 expressed on GI endothelial cells, thus preventing extravascular migration of the T cells into the inflamed gastrointestinal mucosa of IBD patients. Thus, a goal was to achieve maximum blood receptor occupancy (% RO), e.g., greater than 80% RO, greater than 90% RO, or close to 100% RO, in order to block binding and migration of the T cells into the inflamed gastrointestinal mucosa.

In contrast, the present inventors have identified an alternative mechanism by which α4β7 integrin antagonists inhibit inflammation within inflamed tissue, such as inflamed gastrointestinal mucosa, by exerting a local effect. As disclosed in the accompanying Examples, α4β7 integrin antagonist, when present in the inflamed tissue, are able to inhibiting MAdCAM1-mediated CD4+ T cell proliferation and cytokine production that occurs through direct binding and stimulation of α4β7 integrin. It is demonstrated herein that this local effect does not require blood receptor occupancy saturation, but instead, oral administration of a sub-saturating dose of the antagonist is sufficient to achieve a therapeutic effect, e.g., endoscopic improvement or histological improvement. Thus, the disclosure provide, inter alia, methods of treating IBDs that comprise orally providing to a subject a sub-saturating blood receptor occupancy amount of an α4β7 integrin antagonist, including but not limited to the peptide dimer compounds disclosed herein.

In certain aspects, the present disclosure provides methods of using α4β7 antagonist thioether peptide monomers and dimers as anti-inflammatory and/or immunosuppressive agents, e.g., for use in treating a condition that is associated with a biological function of α4β7 or on cells or tissues expressing MAdCAMI1.

Aspects of the invention relate to cyclized, disulfide or thioether peptidic compounds exhibiting integrin antagonist activity, namely, exhibiting high specificity for α4β7 integrin. In certain embodiments, each peptide of the present invention comprises a downstream natural or unnatural amino acid and an upstream modified amino acid or aromatic group that are capable of bridging to form a cyclized structure through a disulfide or thioether bond. Peptides of the present invention demonstrate increased stability when administered orally as a therapeutic agent.

In a further related embodiment, the present invention provides a method for treating or preventing a disease or condition that is associated with a biological function of integrin α4β7, the method comprising providing to a subject in need thereof an effective amount of a peptide molecule of the invention or a pharmaceutical composition of the invention. In certain embodiments, the disease or condition is an inflammatory bowel disease. In particular embodiments, the inflammatory bowel disease is ulcerative colitis or Crohn's disease. In particular embodiments, the peptide molecule inhibits binding of α4β7 to MAdCAM1. In certain embodiments, the peptide molecule or the pharmaceutical composition is provided to the subject in need thereof at an interval sufficient to ameliorate the condition. In certain embodiments, the interval is selected from the group consisting of around the clock, hourly, every four hours, once daily, twice daily, three times daily, four times daily, every other day, weekly, bi-weekly, and monthly. In particular embodiments, the peptide molecule or pharmaceutical composition is provided as an initial does followed by one or more subsequent doses, and the minimum interval between any two doses is a period of less than 1 day, and wherein each of the doses comprises an effective amount of the peptide molecule. In particular embodiments, the effective amount of the peptide molecule or the pharmaceutical composition is sufficient to achieve at least one of the following: a) about 50% or greater saturation of MAdCAM1 binding sites on α4β7 integrin molecules; b) about 50% or greater inhibition of α4β7 integrin expression on the cell surface; and c) about 50% or greater saturation of MAdCAM1 binding sites on α4β7 molecules and about 50% or greater inhibition of α4β7 integrin expression on the cell surface, wherein i) the saturation is maintained for a period consistent with a dosing frequency of no more than twice daily; ii) the inhibition is maintained for a period consistent with a dosing frequency of no more than twice daily; or iii) the saturation and the inhibition are each maintained for a period consistent with a dosing frequency of no more than twice daily. In certain embodiments, the peptide molecule is administered orally, parenterally, or topically.

### Definitions

As used herein, the singular forms “a,” “and” and “the” include plural references unless the context clearly dictates otherwise.

When the term “comprising” is used herein, it is understood that the present invention also includes the same embodiments wherein the term “comprising” is substituted with “consisting essentially of” or “consisting of.”

As used in the present specification the following terms have the meanings indicated:

The term “peptide,” as used herein, refers broadly to a structure comprising a sequence of two or more amino acids joined together by peptide bonds. In particular embodiments, it refers to a sequence of two or more amino acids joined together by peptide bonds. It should be understood that this term does not connote a specific length of a polymer of amino acids, nor is it intended to imply or distinguish whether the polypeptide is produced using recombinant techniques, chemical or enzymatic synthesis, or is naturally occurring. The term “peptide”, as used generically herein, includes both peptide monomers and peptide dimers.

The term “monomer” as used herein may also be referred to as “peptide monomer,” “peptide monomer molecule,” or “monomer peptide.” The term “monomer” indicates a single sequence of two or more amino acids joined together by peptide bonds.

The term “dimer,” as used herein, refers broadly to a peptide comprising two monomer peptide subunits (e.g., thioether monomer peptides) that are linked at their respective C- or N-terminuses. Dimers of the present invention may include homodimers or heterodimers that function as integrin antagonists. The term “dimer” may also be referred to herein to as a “peptide dimer,” “peptide dimer molecule,” “dimer peptide,” or “dimer compound.” The term “monomer peptide subunit” may also be referred to herein as “monomer subunit,” “peptide monomer subunit,” “peptide subunit,” “peptide dimer subunit,” “dimer subunit,” “monomeric subunit,” or “subunit of a peptide dimer.”

The term “thioether,” as used herein, refers to a cyclized, covalent bond formed between an upstream amino acid or aromatic acid group, and a downstream sulfur-containing amino acid, or isostere thereof, i.e., a C—S bond.

The term “linker,” as used herein, refers broadly to a chemical structure that is capable of linking together two thioether monomer subunits to form a dimer.

The term “L-amino acid,” as used herein, refers to the “L” isomeric form of a peptide, and conversely the term “D-amino acid” refers to the “D” isomeric form of a peptide. The amino acid residues described herein are preferred to be in the “L” isomeric form, however, residues in the “D” isomeric form can be substituted for any L-amino acid residue, as long as the desired functional is retained by the peptide.

Unless otherwise indicated, the term “NH₂,” as used herein, refers to the free amino group present at the amino terminus of a polypeptide. The term “OH,” as used herein, refers to the free carboxy group present at the carboxy terminus of a peptide. Further, the term “Ac,” as used herein, refers to Acetyl protection through acylation of the N-terminus of a polypeptide. Where indicated, “NH₂” refers to a free amino group side chain of an amino acid. Where indicated, the term “Ac,” as used herein refers to acylation of an amino acid with NH₂ group.

The term “carboxy,” as used herein, refers to —CO₂H.

The term “isotere” or “isostere replacement,” as used herein, refers to any amino acid or other analog moiety having chemical and/or structural properties similar to a specified amino acid. In particular embodiments, an “isostere” or “suitable isostere” of an amino acid is another amino acid of the same class, wherein amino acids belong to the following classes based on the propensity of the side chain to be in contact with polar solvent like water: hydrophobic (low propensity to be in contact with water), polar or charged (energetically favorable contact with water). The charged amino acid residues include lysine (+), arginine (+), aspartate (−) and glutamate (−). Polar amino acids include serine, threonine, asparagine, glutamine, histidine and tyrosine. The hydrophobic amino acids include alanine, valine, leucine, isoleucine, proline, phenylalanine, tryptophane, cysteine and methionine. The amino acid glycine does not have a side chain and is hard to assign to one of the above classes. However, glycine is often found at the surface of proteins, often within loops, providing high flexibility to these regions, and an isostere may have a similar feature. Proline has the opposite effect, providing rigidity to the protein structure by imposing certain torsion angles on the segment of the polypeptide chain.

The term “cyclized,” as used herein, refers to a reaction in which one part of a polypeptide molecule becomes linked to another part of the polypeptide molecule to form a closed ring, such as by forming a disulfide or thioether bond. In particular embodiments, peptide monomers and monomer subunits of peptide dimers of the present invention are cyclized via an intramolecular disulfide or thioether bond.

The term “receptor,” as used herein, refers to chemical groups of molecules on the cell surface or in the cell interior that have an affinity for a specific chemical group or molecule. Binding between peptide molecules and targeted integrins can provide useful diagnostic tools.

The term “integrin-related diseases,” as used herein, refer to indications that manifest as a result of integrin binding, and which may be treated through the administration of an integrin antagonist.

The term “pharmaceutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

The term “N(alpha)Methylation”, as used herein, describes the methylation of the alpha amine of an amino acid, also generally termed as an N-methylation.

The term “acylating organic compounds,” as used herein refers to various compounds with carboxylic acid functionality, which may be used to acylate the C- and/or N-termini of a peptide molecule. Non-limiting examples of acylating organic compounds include cyclopropylacetic acid, 4-Fluorobenzoic acid, 4-fluorophenylacetic acid, 3-Phenylpropionic acid, Succinic acid, Glutaric acid, Cyclopentane carboxylic acid, glutaric acid, succinic acid, 3,3,3-trifluoropropeonic acid, 3-Fluoromethylbutyric acid.

All peptide sequences are written according to the generally accepted convention whereby the α-N-terminal amino acid residue is on the left and the α-C-terminal is on the right. As used herein, the term “α-N-terminal” refers to the free α-amino group of an amino acid in a peptide, and the term “α-C-terminal” refers to the free α-carboxylic acid terminus of an amino acid in a peptide.

The term “amino acid” or “any amino acid” as used here refers to any and all amino acids, including naturally occurring amino acids (e.g., α-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids are pyrrolysine (found in methanogenic organisms and other eukaryotes), selenocysteine (present in many noneukaryotes as well as most eukaryotes), and N-formylmethionine (encoded by the start codon AUG in bacteria, mitochondria and chloroplasts). “Unnatural” or “non-natural” amino acids are non-proteinogenic amino acids (i.e., those not naturally encoded or found in the genetic code) that either occur naturally or are chemically synthesized. Over 140 natural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

Generally, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. Some abbreviations useful in describing the invention are defined below in the following Table 1.

TABLE 1
Abbreviations

| Abbreviation | Definition |
|---|---|
| DIG | DIGlycolic acid (Linker) |
| Dap | Diaminopropionic acid |
| Dab | Diaminobutyric acid |
| Pen | Penicillamine |
| Sar | Sarcosine |
| Cit | Citroline |
| Cav | Cavanine |
| 4-Guan | 4-Guanidine-Phenylalanine |
| N-Me-Arg; N(alpha)Methylation | N-Methyl-Arginine |
| Ac- | Acetyl |
| 2-Nal | 2-Napthylalanine |
| 1-Nal | 1-Napthylalanine |
| Bip | Biphenylalanine |
| O—Me-Tyr | Tyrosine (O-Methyl) |
| N-Me-Lys | N-Methyl-Lysine |
| N-Me-Lys (Ac) | N-Me-Acetyl (ε) Lysine |
| Ala (3,3 diphenyle) | 3,3 diphenyl alanine |
| NH₂ | Free Amine |
| CONH₂ | Amide |
| COOH | Acid |
| Phe (4-F) | 4-Fluoro-Phenylanine |
| PEG13 | Bifunctional PEG linker with 13 PolyEthylene |
| | Glycol units |
| PEG25 | Bifunctional PEG linker with 25 PolyEthylene |
| | Glycol units |
| PEG1K | Bifunctional PEG linker with PolyEthylene |
| | Glycol Mol wt of 1000 Da |
| PEG2K | Bifunctional PEG linker with PolyEthylene |
| | Glycol Mol wt of 2000 Da |
| PEG3.4K | Bifunctional PEG linker with PolyEthylene |
| | Glycol Mol wt of 3400 Da |
| PEG5K | Bifunctional PEG linker with PolyEthylene |
| | Glycol Mol wt of 5000 Da |
| IDA | β-Ala-Iminodiacetic acid (Linker) |
| IDA-Palm | β-Ala (Palmityl)-Iminodiacetic acid |
| HPhe | Homo Phenylalanine |
| Ahx | Aminohexanoic acid |
| DIG-OH | Glycolic monoacid |
| Triazine | Amino propyl Triazine di-acid |
| Boc-Triazine | Boc-Triazine di-acid |
| Trifluorobutyric acid | Acylated with 4,4,4-Trifluorobutyric acid |
| 2-Methly-trifluorobutyric acid | acylated with 2-methy-4,4,4-Butyric acid |
| Trifluorpentanoic acid | Acylated with 5,5,5-Trifluoropentnoic acid |
| 1,4- Phenylenediacetic acid | para- Phenylenediacetic acid (Linker) |
| 1,3 - Phenylenediacetic acid | meta - Phenylenediacetic acid (Linker) |
| DTT | Dithiothreotol |
| Nle | Norleucine |
| β-HTrp | β-homoTrypophane |
| β-HPhe | β-homophenylalanine |
| Phe(4-CF₃) | 4-Trifluoromethyl Phenylalanine |
| β-Glu | 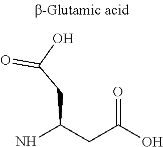 |
| β-HGlu beta-Homo-Glu | 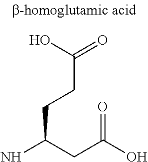 [SMILES: N[C@@H](CCC(=O)O)CC(=O)O] |
| 2-2-Indane | 2-Aminoindane-2-carboxylic acid |
| 1-1-Indane | 1-Aminoindane-1-carboxylic acid |
| Cpa | Cyclopentyl alanine |
| Orn | Ornithine |
| Aoc | 2-Amono octonoic acid |
| Cba | Cyclibutyl alanine |
| HCha | homocyclohexyl Alanine |
| Cyclobutyl | Cyclobutylalanine |
| β-HPhe, B-H-K | β-homophenylalanine |
| HLeu, homo-Leu, hK, | Homoleucine |
| Gla | Gama-Carboxy-Glutamic acid |
| Tic | 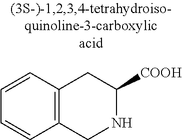 [SMILES: O=C(O)[C@@H]1Cc2ccccc2CN1] |
| Phe(4CF3) | L-Phe(4CF₃)—OH |
| | Phe(4-trifluoromethyl |
| | 3-(4-trifluoromethyl-phenyl)propionic acid |
| Phe(2,4-diCl) | (S)- 2-amino-3-(2,4-dichlorophenyl)propionic acid |
| Phe(3,4-diCl) | (S)- 2-amino-3-(3,4-dichlorophenyl)propionic acid |
| Pen(═O) | Penicillamine sulfoxide |
| Aic | aminoindan-2-carboxylic acid |
| Phe(2-carbomyl) | L-2-carbamoylphenylalanine |
| Phe(3-carbomyl) | L-3-carbamoylphenylalanine |
| Phe(4-COOH) | (4-carboxy-tert-butyl)-L-phenylalanine |
| Phe(4-Ome) | (S)-4-methoxyphenylalanine |
| Phe(4tBu) | (S) -2-amino-3-(4-tertbutyl-phenyl)propionic acid |
| Phe(4F) | 4-fluoro-L-phenylalanine |
| Glu(OMe) | L-glutamic acid g-methyl ester |
| alpha-bromobutyryl | 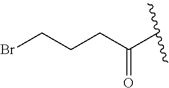 [SMILES: *C(=O)CCCBr] |
| alpha-bromopropenyl; Propionyl | 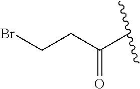 [SMILES: *C(=O)CCBr] |
| alpha-bromoisobutyryl | 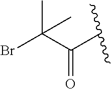 [SMILES: *C(=O)C(C)(C)Br] |
| alpha-H-E; alpha-hGlu | 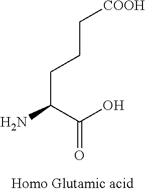 [SMILES: N[C@@H](CCCC(=O)O)C(=O)O] |
| F(2-Me) | 2-Methyl Phenylalanine |
| 4-Benzyl | 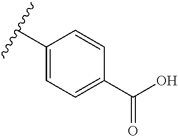 [SMILES: *c1ccc(C(=O)O)cc1] |
| 2-Benzyl | 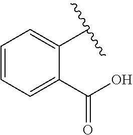 [SMILES: *c1ccccc1C(=O)O] |
| 3-Benzyl | 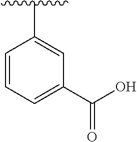 [SMILES: *c1cccc(C(=O)O)c1] |
| erythro-b-F-S | 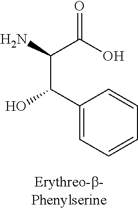 [SMILES: N[C@@H](C(=O)O)[C@@H](O)c1ccccc1] |
| Threo-b-F-S | 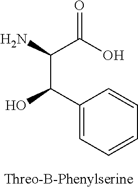 [SMILES: N[C@@H](C(=O)O)[C@H](O)c1ccccc1] |
| F(2-CF3) | 2-Trifluoromethyl-Phenylalanine |
| F(CF3) | 4-Trifluoromethyl-Phenylalinine |
| F(4-Me); 4-Me—F | 4-Methyl Phenylalanine |
| F(3-Me) | 3-Methyl Phenylalanine |
| Alpha-hGlu | HomoGlutamc acid |
| ATC | 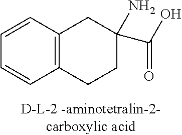 [SMILES: NC1(C(=O)O)CCc2ccccc2C1] |
| BPA | 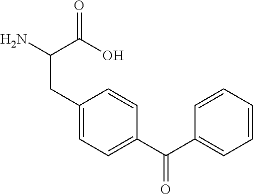 [SMILES: NC(Cc1ccc(C(=O)c2ccccc2)cc1)C(=O)O] |
| b-Me—F | 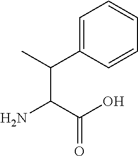 |
| β-dimethyl—F | 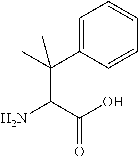 |
| 2-Chloro Benzoyl | 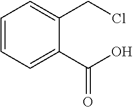 [SMILES: O=C(O)c1ccccc1CCl] |
| N-Me-E | N-Methyl Glutamic acid |
| k(Ac) | Nε-Acety -D-Lysine |
| k(PEG8) | PEG8 conjugated (Nε)-D-Lys |
| N-Me-k(Ac) | N-methyl Nε-Acetyl -D-Lysine |
| N-Me-K(Ac) | N-methyl Nε-Acetyl -Lysine |
| F(4-tBu); F(4tBu) | 4-tButyl-Phenylalanine |
| C(thioether propane) | S—CH2—CH2—CH2—S |
| l(D-L) | D-leucine |
| β-azido-Ala—OH | β-azido-Alanine |
| Aoc | 8-amino-octanoic acid |

### Peptide Antagonists

The present invention relates generally to cyclic peptides, e.g., disulfide and thioether peptides, that have been shown to have integrin antagonist activity. In particular, the present invention relates to various peptides that form cyclized structures through intramolecule bonds, e.g., disulfide or thioether bonds, e.g., intramolecular disulfide or thioether bonds. While the disclosure provided herein is generally directed to peptides having disulfide or thiether intramolecular bonds, it is understood that other cyclic peptide antagonists of a4b7 integrin, including those comprising intramolecular bonds of a different nature, and also cyclic peptide antagonists of α4β7 integrin comprising bonds between two peptide monomer subunits, may also be used to practice the methods disclosed herein. Certain embodiments relate to disulfide or thioether peptide monomers with integrin antagonist activity. Some embodiments relate to disulfide or thioether peptide dimers with integrin antagonist activity comprising hetero- or homo-monomer thioether peptide subunits, wherein the disulfide or thioether peptide subunits are linked at either their C- or N-terminuses. The cyclized structure of the peptides, peptide monomers or peptide subunits have been shown to increase the potency, selectivity, and stability of the peptide molecules, as discussed below. In some embodiments, dimerizing the peptide monomer increases potency, selectivity, and/or stability compared to a non-dimerized peptide. Illustrative peptides and genuses thereof that may be used according to the methods disclosed herein are provided in the following patent application publications, each of which is incorporated by reference in its entirety: PCT Application Publication Nos. WO 2014/059213, WO 2014/165448, WO 2014/165449, WO 2015/176035, WO 2016/054411, and WO 2016/054445.

In some instances, the monomer peptides further comprise C- and/or N-termini that comprise free amine (or both C- and N-termini that comprise free amine). Similarly, a peptide dimer may comprise one or more C- or N-termini that comprise a free amine. Thus, a user may modify either terminal end to include a modifying group such as a PEGylation, e.g., a small PEGylation (e.g. PEG4-PEG13). A user may further modify either terminal end through acylation. For example, in some instances at least one of the N- and C-terminus of a peptide molecule is acylated with an acylating organic compound selected from the group consisting of 2-Me-Trifluorobutyl, Trifluoropentyl, Acetyl, Octonyl, Butyl, Pentyl, Hexyl, Palmityl, Trifluoromethyl butyric, cyclopentane carboxylic, cyclopropylacetic, 4-fluorobenzoic, 4-fluorophenyl acetic, 3-Phenylpropionic acid. In some instances, peptide molecules of the instant invention comprise both a free carboxy terminal and a free amino terminal, whereby a user may selectively modify the peptide to achieve a desired modification. It is further understood that the C-terminal residues of the thioether peptides, e.g., thioether monomers, disclosed herein are amides or acids, unless otherwise indicated. One having skill in the art will therefore appreciate that the thioether peptides of the instant invention may be selectively modified, as desired.

With respect to peptide dimers, it is understood that monomer subunits are dimerized to form peptide dimer molecules, e.g., the monomer subunits are joined or dimerized by a suitable linker moiety, as defined herein. Some of the monomer subunits are shown having C- and N-termini that both comprise free amine. Thus, a user may modify either terminal end of the monomer subunit to eliminate either the C- or N-terminal free amine, thereby permitting dimerization at the remaining free amine. Thus, some of the monomer subunits comprise both a free carboxy or amide at C-terminal and a free amino terminal, whereby a user may selectively modify the subunit to achieve dimerization at a desired terminus. One having skill in the art will therefore appreciate that the monomer subunits of the instant invention may be selectively modified to achieve a single, specific amine for a desired dimerization.

It is further understood that the C-terminal residues of the monomer subunits disclosed herein comprises —OH or —NH₂, unless otherwise indicated. Further, it is understood that dimerization at the C-terminal may be facilitated by using a suitable amino acid with a side chain having amine functionality, as is generally understood in the art. In particular embodiments, a linker binds to functional amine groups in the C-terminal amino acid of each of the peptide monomer subunits to form a dimer. Regarding the N-terminal residues, it is generally understood that dimerization may be achieved through the free amine of the terminal residue, or may be achieved by using a suitable amino acid side chain having a free amine, as is generally understood in the art.

The peptide monomers and dimers of the instant invention, or peptide subunits thereof, may further comprise one or more terminal modifying groups. In at least one embodiment, a terminal end of a peptide is modified to include a terminal modifying group selected from the non-limiting group consisting of DIG, PEG4, PEG13, PEG25, PEG1K, PEG2K, PEG4K, PEG5K, Polyethylene glycol having molecular weight from 400 Da to 40,000 Da, PEG having a molecular weight of 40,000 Da to 80,000 Da, IDA, ADA, Glutaric acid, Succinic acid, Isophthalic acid, 1,3-phenylenediacetic acid, 1,4-phenylenediacetic acid, 1,2-phenylenediacetic acid, AADA, and suitable aliphatics, aromatics, and heteroaromatics.

In some embodiments of the peptide dimers, peptide dimer subunits or peptide monomers described herein, the N-terminus further comprises a suitable linker moiety or other modifying group. In some embodiments of peptide monomers described herein, the N-terminus may further be acylated.

Non-limiting examples of terminal modifying groups are provided in Table 2.

TABLE 2
Illustrative Terminal Modifying Groups

| Abbreviation | Description | Structure |
|---|---|---|
| DIG | DIGlycolic acid, | 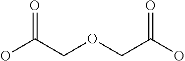 |
| PEG4 | Bifunctional PEG linker with 4 PolyEthylene Glycol units | 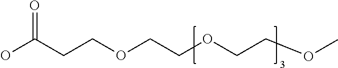 |
| PEG13 | PEG with 13 PolyEthylene Glycol units | 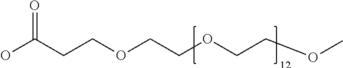 |
| PEG25 | PEG with 25 PolyEthylene Glycol units | 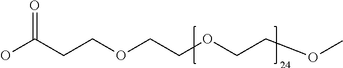 |
| PEG1K | PolyEthylene Glycol Mol | |
| | wt of 1000 Da | |
| PEG2K | PolyEthylene Glycol Mol | |
| | wt of 2000 Da | |
| PEG3.4K | PolyEthylene Glycol Mol | |
| | wt of 3400 Da | |
| PEG5K | PolyEthylene Glycol Mol | |
| | wt of 5000 Da | |
| DIG | DIGlycolic acid, | 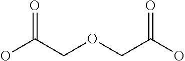 |
| IDA | β-Ala-Iminodiacetic acid | 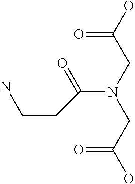 [SMILES: NCCC(=O)N(CC(=O)O)CC(=O)O] |
| Boc-IDA | Boc-β-Ala-Iminodiacetic acid | 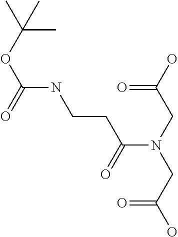 [SMILES: CC(C)(C)OC(=O)NCCC(=O)N(CC(=O)O)CC(=O)O] |
| Ac-IDA | Acetyl-β-Ala-Iminodiacetic acid | 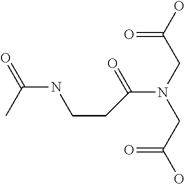 [SMILES: CC(=O)NCCC(=O)N(CC(=O)O)CC(=O)O] |
| GTA | Glutaric acid | 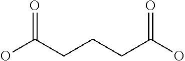 |
| PMA | Pemilic acid | 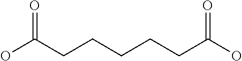 [SMILES: O=C(O)CCCCCC(=O)O] |
| AZA | Azelaic acid | 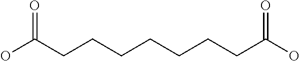 [SMILES: O=C(O)CCCCCCCC(=O)O] |
| DDA | Dodecanedioic acid | 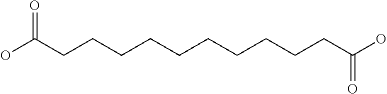 [SMILES: O=C(O)CCCCCCCCCCC(=O)O] |
| ADA | Amino diacetic acid | 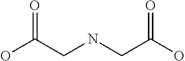 |
| AADA | n-Acetyl amino acetic acid | 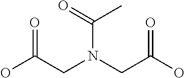 |
| PEG4-Biotin | PEG4-Biotin (Product number 10199, QuantaBioDesign) | 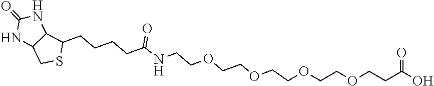 [SMILES: O=C(O)CCOCCOCCOCCOCCNC(=O)CCCCC1SCC2NC(=O)NC21] |

The linker moieties of the instant invention may include any structure, length, and/or size that is compatible with the teachings herein. In at least one embodiment, a linker moiety is selected from the non-limiting group consisting of DIG, PEG4, PEG4-biotin, PEG13, PEG25, PEG1K, PEG2K, PEG3.4K, PEG4K, PEG5K, IDA, ADA, Boc-IDA, Glutaric acid, Isophthalic acid, 1,3-phenylenediacetic acid, 1,4-phenylenediacetic acid, 1,2-phenylenediacetic acid, Triazine, Boc-Triazine, IDA-biotin, PEG4-Biotin, AADA, suitable aliphatics, aromatics, heteroaromatics, and polyethylene glycol based linkers having a molecular weight from approximately 400 Da to approximately 40,000 Da or approximately 40,000 Da to approximately 80,000 Da.

When the linker is IDA, ADA or any linker with free amine it can be acylated with acylating organic compound selected from the group consisting of 2-me-Trifluorobutyl, Trifluoropentyl, Acetyl, Octonyl, Butyl, Pentyl, Hexyl, Palmityl, Lauryl, Oleoyl, Lauryl, Trifluoromethyl butyric, cyclopentane carboxylic, cyclopropylacetic, 4-fluorobenzoic, 4-fluorophenyl acetic, 3-Phenylpropionic, tetrahedro-2H-pyran-4carboxylic, succinic acid, and glutaric acid, straight chain aliphatic acids with 10 to 20 carbon units, cholic acid and other bile acids. In some instances small PEG (PEG4-PEG13), Glu, or Asp is used as spacer before acylations.

In certain embodiments, the linker connects two monomeric subunits by connecting two sulfur containing C- or N-terminal amino acids. In some embodiments, the two sulfur containing amino acids are connected by a linker comprising a di-halide, an aliphatic chain, or a PEG. In certain embodiments, the linker connects two monomeric subunits by connecting sulfur containing C-terminal amino acids at the C-terminus of each monomer subunit. In some embodiments, the two sulfur containing amino acids are connected by a linker comprising homobifunctional maleimide crosslinkers, di-halide, 1,2-Bis(bronornonethyl)benzene, 1,2-Bis(chloromomethyl)benzene, 1,3-Bis(bromomomethyl)benzene, 1,3-Bis(chloromomethyl)benzene, 1,4-Bis(bromomomethyl)benzene, 1,4-Bis(chloromomethyl)benzene, 3,3′-bis-bromomethyl-biphenyl, or 2,2′-bis-bromomethyl-biphenyl. Particular haloacetyl crosslinkers contain an iodoacetyl or a bromoacetyl group. These homobifunctional linkers may contain spacers comprising PEG or an aliphatic chain.

Non-limiting examples of suitable linker moieties are provided in Table 3.

TABLE 3
Illustrative Linker Moieties

| Abbrivation | Discription | Structure |
|---|---|---|
| DIG | DIGlycolic acid, | 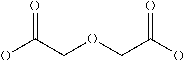 |
| PEG4 | Bifunctional PEG linker with 4 PolyEthylene Glycol units | 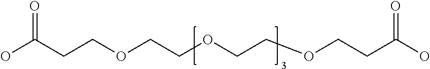 |
| PEG13 | Bifunctional PEG linker with 13 PolyEthylene Glycol units | 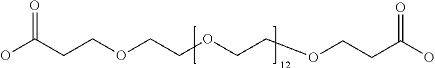 |
| PEG25 | Bifunctional PEG linker with 25 PolyEthylene Glycol units | 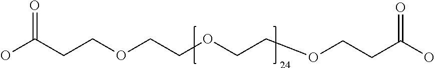 |
| PEG1K | Bifunctional PEG linker with PolyEthylene | |
| | Glycol Mol wt of 1000 Da | |
| PEG2K | Bifunctional PEG linker with PolyEthylene | |
| | Glycol Mol wt of 2000 Da | |
| PEG3.4K | Bifunctional PEG linker with PolyEthylene | |
| | Glycol Mol wt of 3400 Da | |
| PEG5K | Bifunctional PEG linker with PolyEthylene | |
| | Glycol Mol wt of 5000 Da | |
| DIG | DIGlycolic acid, | 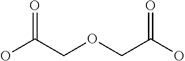 |
| IDA | β-Ala-lminodiacetic acid | 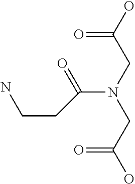 [SMILES: NCCC(=O)N(CC(=O)O)CC(=O)O] |
| Boc-IDA | Boc-β-Ala-Iminodiacetic acid | 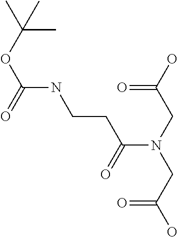 [SMILES: CC(C)(C)OC(=O)NCCC(=O)N(CC(=O)O)CC(=O)O] |
| Ac-IDA | Ac-β-Ala-Iminodiacetic acid | 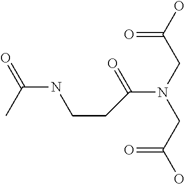 [SMILES: CC(=O)NCCC(=O)N(CC(=O)O)CC(=O)O] |
| IDA-Palm | Palmityl-β-Ala-Iminodiacetic acid | 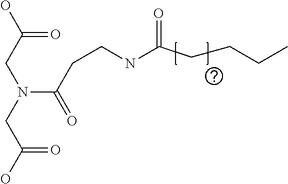 |
| GTA | Glutaric acid | 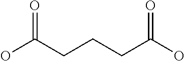 |
| PMA | Pemilic acid | 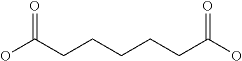 [SMILES: O=C(O)CCCCCC(=O)O] |
| AZA | Azelaic acid | 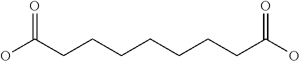 [SMILES: O=C(O)CCCCCCCC(=O)O] |
| DDA | Dodecanedioic acid | 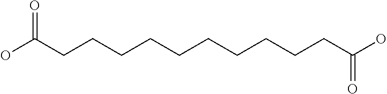 [SMILES: O=C(O)CCCCCCCCCCC(=O)O] |
| IPA | Isopthalic aicd | 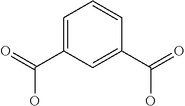 [SMILES: O=C(O)c1cccc(C(=O)O)c1] |
| 1,3-PDA | 1,3- Phenylenediacetic acid | 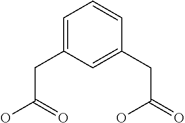 [SMILES: O=C(O)Cc1cccc(CC(=O)O)c1] |
| 1,4-PDA | 1,4- Phenylenediacetic acid | 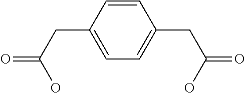 [SMILES: O=C(O)Cc1ccc(CC(=O)O)cc1] |
| 1,2-PDA | 1,2 - Phenylenediacetic acid | 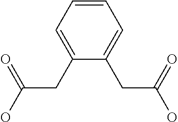 [SMILES: O=C(O)Cc1ccccc1CC(=O)O] |
| Triazine | Amino propyl Triazine di-acid | 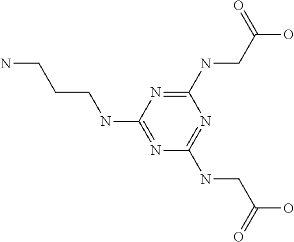 [SMILES: NCCCNc1nc(NCC(=O)O)nc(NCC(=O)O)n1] |
| Boc- Triazine | Boc-Triazine di-acid | 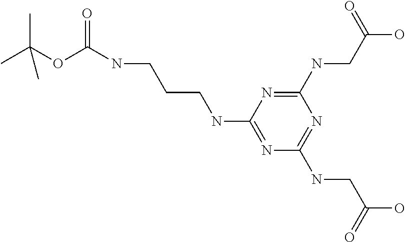 [SMILES: CC(C)(C)OC(=O)NCCCNc1nc(NCC(=O)O)nc(NCC(=O)O)n1] |
| ADA | Amino diacetic acid | 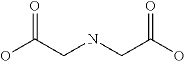 |
| AADA | n-Acetyl amino acetic acid | 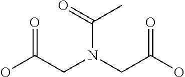 |
| PEG4- Biotin | PEG4-Biotin (Product number 10199, QuantaBioDesign) | 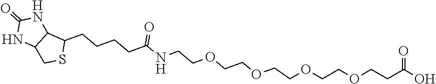 [SMILES: O=C(O)CCOCCOCCOCCOCCNC(=O)CCCCC1SCC2NC(=O)NC21] |
| 1,4 BMB | 1,4-Bis(halo-momethyl)benzene | 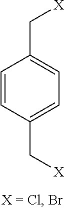 |
| 1,2 BMB | 1,2-Bis(halo-momethyl)benzene | 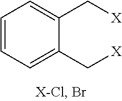 |
| 1,3 BMB | 1,3-Bis(halo-momethyl)benzene, | 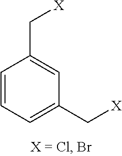 |
| 1,3 BMBip | 3,3′-Bis-Halomethyl-Biphenyl | 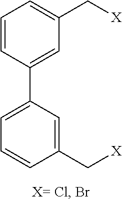 |
| IDA-Biotin | N-Biotin-β-Ala-Iminodiacetic acid | 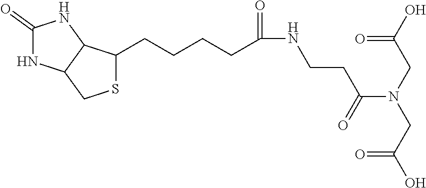 [SMILES: O=C(O)CN(CC(=O)O)C(=O)CCNC(=O)CCCCC1SCC2NC(=O)NC21] |
| 2,2 BMBip | 2,2′-Bis-Halomethyl-Biphenyl | 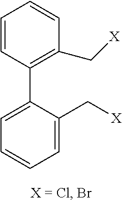 |
| BMal | Bis-Mal-dPEG | 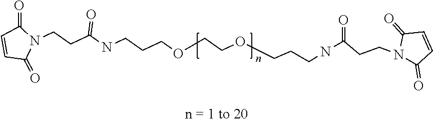 |

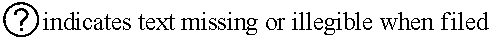

One of skill in the art will appreciate that certain amino acids and other chemical moieties are modified when bound to another molecule. For example, an amino acid side chain may be modified when it forms an intramolecular bridge with another amino acid side chain. In addition, when Homo-Ser-Cl binds to an amino acid such as Cys or Pen via a thioether bond, the Cl moiety is released. Accordingly, as used herein, reference to an amino acid or modified amino acid, such as Homo-Ser-Cl, present in a peptide dimer of the present invention (e.g., at position Xaa⁴ or position Xaa¹⁰) is meant to include the form of such amino acid or modified amino acid present in the peptide both before and after forming the intramolecular bond.

In particular embodiments, methods disclosed herein are practiced using any of the following peptide antagonists of α4β7 integrin, although it is understood that the methods disclosed herein may be practiced using other peptide antagonists, including those disclosed in the PCT applications incorporated by reference herein.

In some embodiments, the peptide antagonist is a peptide dimer compound comprising two peptides, or a pharmaceutically acceptable salt thereof, wherein each of the two peptides comprises or consists of any of the sequences:

(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH;
(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;
(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;
(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;
(SEQ ID NO: 5)
2-methylbenzoyl-(N-Me-Arg)-Ser-Asp-Thr-Leu-Pen-
Phe(4-tBu)-(β-homo-Glu)-(D-Lys)-OH;
or
(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂;
(SEQ ID NO: 6)
Pen-(N-Me-Arg)-Ser-Asp-Thr-Leu-Pen-Phe(4-tBu)-
(β-homo-Glu)-(D-Lys)-OH;
(SEQ ID NO: 6)
Pen-(N-Me-Arg)-Ser-Asp-Thr-Leu-Pen-Phe(4-tBu)-
(β-homo-Glu)-(D-Lys)-NH2;
(SEQ ID NO: 7)
Pen-(N-Me-Arg)-Ser-Asp-Thr-Leu-Pen-Phe(4-tBu)-
(β-homo-Glu)-Pro-(D-Lys)-OH;
(SEQ ID NO: 7)
Pen-(N-Me-Arg)-Ser-Asp-Thr-Leu-Pen-Phe(4-tBu)-
(β-homo-Glu)-Pro-(D-Lys)-NH2;
(SEQ ID NO: 8)
Pen-(N-Me-Arg)-Ser-Asp-Thr-Leu-Pen-Phe(4-tBu)-
(β-homo-Glu)-(D-Pro)-(D-Lys)-OH;
or
(SEQ ID NO: 8)
Pen-(N-Me-Arg)-Ser-Asp-Thr-Leu-Pen-Phe(4-tBu)-
(β-homo-Glu)-(D-Pro)-(D-Lys)-NH2;

wherein each of the two peptides comprises a thioether bond between the 2-methylbenzoyl and the Pen; or a disulfide between the two Pens; wherein the two peptides are linked by a linker moiety bound to the D-Lys amino acids of the two peptides, and wherein the linker moiety is diglycolic acid (DIG). The peptides may also include an N-terminal Ac.

In particular embodiments of any of the peptide antagonists or pharmaceutically acceptable salts thereof, the pharmaceutically acceptable salt of the peptide dimer compound is an acetate salt.

In certain embodiments, each of the two peptides consists of the sequence:

(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, each of the two peptides consists of the sequence:

(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, each of the two peptides consists of the sequence:

(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, each of the two peptides comprises or consists of the sequence:

(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, each of the two peptides comprises or consists of the sequence.

(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, each of the two peptides comprises or consists of the sequence:

(SEQ ID NO: 5)
2-methylbenzoyl-(N-Me-Arg)-Ser-Asp-Thr-Leu-Pen-
Phe(4-tBu)-(β-homo-Glu)-(D-Lys)-OH,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, the peptide dimer compound or pharmaceutically acceptable salt thereof is:

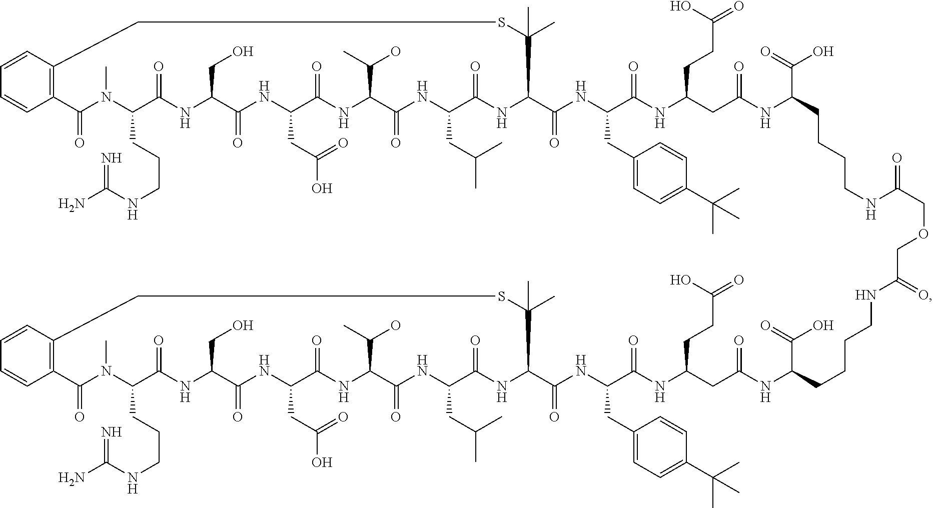 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

or a pharmaceutically acceptable salt thereof.

In certain embodiments, the peptide dimer compound or pharmaceutically acceptable salt thereof is:

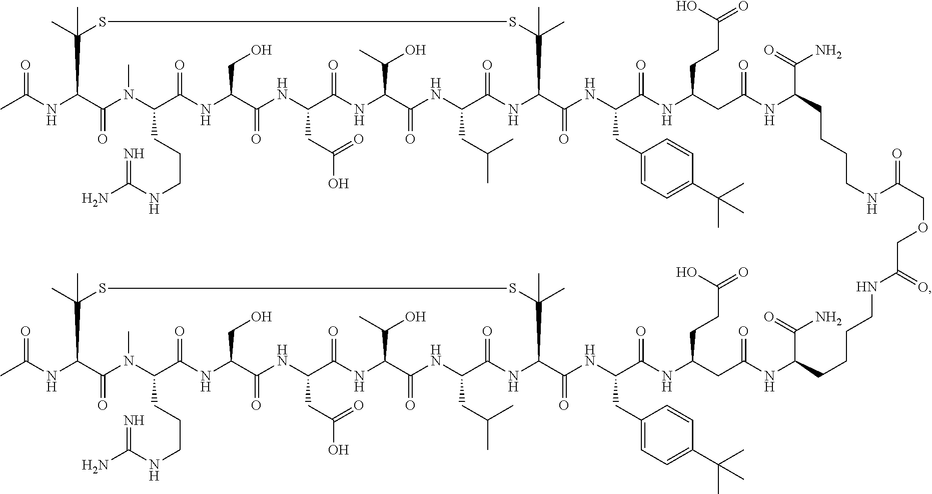 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)[C@@H](NC(C)=O)C(C)(C)SSC2(C)C)C(N)=O)C(N)=O)C(C)(C)SSC1(C)C]

or a pharmaceutically acceptable salt thereof.

In certain embodiments, each of the two peptides comprises or consists of the sequence: 2-methylbenzoyl-(N-Me-Arg)-Ser-Asp-Thr-Leu-Pen-Phe(4-tBlu)-(β-homo-Glu)-(D-Glu)-(D-Lys)-OH (SEQ ID NO: 1),

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, each of the two peptides comprises or consists of the sequence:

(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, each of the two peptides comprises or consists of the sequence:

(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, each of the two peptides comprises or consists of the sequence:

(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, each of the two peptides comprises or consists of the sequence:

(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂;

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, each of the two peptides comprises or consists of the sequence:

(SEQ ID NO: 5)
2-methylbenzoyl-(N-Me-Arg)-Ser-Asp-Thr-Leu-Pen-
Phe(4-tBu)-(β-homo-Glu)-(D-Lys)-OH,

wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

In certain embodiments, the peptide dimer compound or pharmaceutically acceptable salt thereof is:

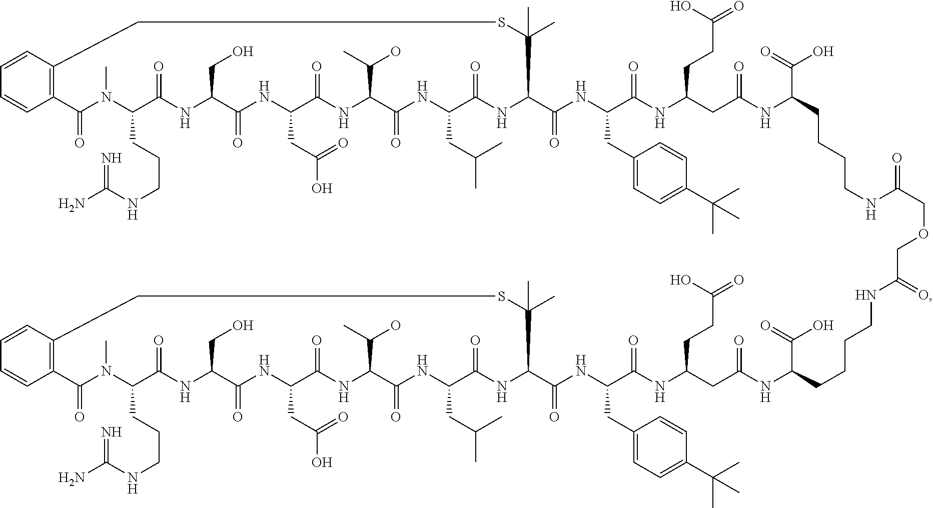 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

or a pharmaceutically acceptable salt thereof.

In certain embodiments, the peptide dimer compound or pharmaceutically acceptable salt thereof is:

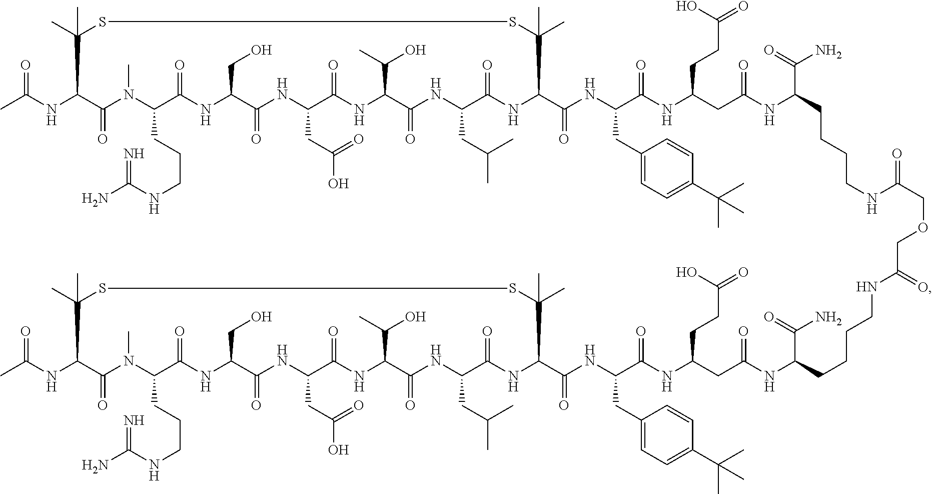 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)[C@@H](NC(C)=O)C(C)(C)SSC2(C)C)C(N)=O)C(N)=O)C(C)(C)SSC1(C)C]

or a pharmaceutically acceptable salt thereof.

In particular embodiments, the peptide dimer compound is Compound A or Compound B, as described in the accompanying Examples.

Peptide Biological Activity

In certain embodiments, the peptide molecules disclosed herein have increased affinity for α4β7 binding, increased selectivity against α4β1, and increased stability in simulated intestinal fluid (SIF) as well as in gastric environment under reduced conditions. These novel antagonist molecules demonstrate high binding affinity with α4β7, thereby preventing binding between α4β7 and the MAdCAM1 ligand. Accordingly, these peptide molecules have shown to be effective in eliminating and/or reducing the inflammation process in various experiments.

The peptide monomer and dimer molecules bind or associate with the α4β7 integrin to disrupt or block binding between α4β7 and the MAdCAM1 ligand. In certain embodiments, peptide dimer and monomer molecules of the present invention inhibit or reduce binding between α4β7 and the MAdCAM1 ligand. In certain embodiments, a peptide of the present invention reduces binding of α4β7 and the MAdCAM1 ligand by at least 20%, at least 30%, at least 40%, at least 50%, at least 60%, at least 70%, at least 80%, or at least 90% as compared to a negative control peptide. Methods of determining binding are known in the art and described herein, and include ELISA assays, for example.

In certain embodiments, a peptide monomer or dimer molecule has an IC50 of <500 nM, <250 nM, <100 nM, <50 nM, <25 nM, or <10 nM. Methods of determining activity are known in the art and include any of those described in the accompanying Examples.

In some embodiments, a peptide monomer or dimer molecule has a half-life of greater than 180 minutes when exposed to simulated intestinal fluids (SIF). Some implementations further provide a peptide monomer or dimer molecule comprising a half-life from approximately 1 minute to approximately 180 minutes. Similarly these peptides are stable to gastric environment under reduced conditions with half-life >120 min when tested in DTT (Dithiothreitol) assay.

In certain embodiments, a peptide monomer or dimer molecule has increased stability, increased gastrointestinal stability, and/or increased stability in stimulated intestinal fluid (SIF), as compared to a control peptide. In particular embodiments, a control peptide is a peptide having the identical or a highly related amino acid sequence (e.g., >90% sequence identity) as the peptide monomer or dimer molecule, but which does not form a cyclized structure through a thioether bond. In some embodiments relating to dimer molecules, the control peptide is not dimerized. In particular embodiments, the only difference between the peptide monomer or dimer molecule and the control peptide is that the peptide comprises one or more amino acid substitutions that introduce one or more amino acid residues into the peptide, wherein the introduced residue(s) forms a thioether bond with another residue in the peptide.

Methods of determining the stability of a peptide are known in the art. In certain embodiments, the stability of a peptide (e.g. a peptide monomer or dimer as described herein) is determined using an SIF assay, e.g., as described in the accompanying Examples. In particular embodiments, a peptide monomer or dimer molecule of the present invention has a half-life under a given set of conditions (e.g., temperature) of greater than 1 minute, greater than 10 minutes, greater than 20 minutes, greater than 30 minutes, greater than 60 minutes, greater than 90 minutes, greater than 120 minutes, greater than 3 hours, or greater than four hours when exposed to SIF. In certain embodiments, the temperature is about 25° C., about 4° C., or about 37° C., and the pH is a physiological pH, or a pH about 7.4.

In some embodiments, the half-life is measured in vitro using any suitable method known in the art, e.g., in some embodiments, the stability of a peptide monomer or dimer molecule of the present invention is determined by incubating the peptide with pre-warmed human serum (Sigma) at 37° C. Samples are taken at various time points, typically up to 24 hours, and the stability of the sample is analyzed by separating the peptide monomer or dimer from the serum proteins and then analyzing for the presence of the peptide monomer or dimer of interest using LC-MS.

In certain embodiments, peptide dimer or monomer molecules inhibit or reduce α4β7-mediated inflammation. In related embodiments, peptide monomers or dimers of the present invention inhibit or reduce α4β7-mediated secretion or release of one or more cytokines (including any disclosed herein) by T cells, e.g., T cells in the GI mucosa responding to MAdCAM1. Methods of determining inhibition of cytokine secretion and inhibition of signaling molecules are known in the art.

In certain embodiments, peptide monomer or dimer molecules demonstrate increased binding selectivity. In certain instances, peptide monomers or dimers binds to α4β7 with at least a two-fold, three-fold, five-fold, or ten-fold greater affinity than the monomers or dimers bind to α4β1.

In some embodiments, the peptide monomer or dimer molecules demonstrate increased potency as a result of substituting various natural amino acyl residues with N-methylated analog residues. In particular embodiments, potency is measured as IC50 of binding to α4β7, e.g., determined as described herein, while in some embodiments, potency indicates functional activity, e.g., according to a cell adhesion assay.

In particular embodiments, any of these superior characteristics of the peptides of the present invention are measured as compared to a control peptide.

Methods of Manufacture

The peptides (e.g. peptide monomers or peptide dimers) of the present invention may be synthesized by techniques that are known to those skilled in the art, e.g., as disclosed in PCT Application Publication Nos. WO 2014/059213, WO 2014/165448, WO 2014/165449, WO 2015/176035, WO 2016/054411, or WO 2016/054445. Such techniques include the use of commercially available robotic protein synthesizers (e.g. Symphony multiplex peptide synthesizer from Protein Technologies). In some embodiments, novel peptide monomers or dimer subunits are synthesized and purified using techniques described herein.

Methods of Treatment and Pharmaceutical Compositions

In some embodiments, the present invention provides methods for treating an individual or subject afflicted with a condition or indication characterized by α4β7 integrin binding, e.g., to MAdCAM1, wherein the methods comprise providing or administering to the individual or subject an integrin antagonist, e.g., a peptide molecule, described herein. In particular embodiments, subjects or individuals are mammals, e.g., humans or non-human mammals, such as a dog, cat or horse. It is understood that the integrin antagonist may be present in a pharmaceutical composition, e.g., any of those disclosed herein. It is further understood that other agents that inhibit disrupt α4β7 integrin or MAdCAM1 signaling, or α4β7 integrin binding, e.g., to MAdCAM1, may be used as alternatives to the antagonists disclosed herein.

In certain embodiments of the disclosed methods, the method reduces cell surface expression of 37 on CD4+ T cells in the gastrointestinal tract.

In certain embodiments of the disclosed methods, the method inhibits MadCAM1-mediated T cell proliferation in the gastrointestinal tract.

In certain embodiments of the disclosed methods, the method reduces cell surface expression of 37 on CD4+ T cells in the gastrointestinal tract.

In certain embodiments of the disclosed methods, the method induces internalization of α4β7 integrin on CD4+ T memory cells.

In certain embodiments of the disclosed methods, the method causes reduced adhesion of CD4+ T memory cells to MAdCAM1 in the gastrointestinal tract.

In certain embodiments of the disclosed methods, the method inhibits homing of T cells to the gastrointestinal tract, optionally to the ileal lamina propia and/or Peyer's Patches.

In certain embodiments of the disclosed methods, the method is used to treat an IBD, optionally wherein the IBD is ulcerative colitis or Crohn's disease.

In certain embodiments of the disclosed methods, the method results in one or more of the following pharmacokinetic parameters in plasma of the subject:
Cmax (ng/mL) of 1-25, optionally 4-12;
Tmax (h) of 1-5, optionally 2-4;
AUCₜ (ng·h/mL) of 10-250, optionally 50-150;
AUC_{inf} (ng·h/mL) of 10-300, optionally 30-250;
t_{1/2} (h) of 3-10, optionally 4-10;
AUCₜₐᵤ (ng·h/mL) of 30-130;
Ctrough (ng/mL) of 1-5;
accumulation Cmax (ng·mL) of 0.5-2.5, optionally 2-3; and
accumulation AUCₜ (ng·h/mL) of 0.5-3.0.
   In particular embodiments of these methods, the method comprises providing an antagonist disclosed herein, optionally Compound A or Compound A, orally, at a dose of about 150 mg twice a day or about 450 mg twice a day.

In certain embodiments of the disclosed methods, the method results in one or more of the following pharmacodynamic parameters in plasma of the subject:
ROmax (%) of 50-100, optionally 90-100;
average RO (%) of 50-95, optionally 65-95;
change in receptor expressionₘₐₓ (%) of −20 to −60, optionally −35 to −60;
average change in receptor expression (%) of −10 to −55, optionally −25 to −55;
steady state ROmax (%) of 80-100;
average RO₀₋₂₄ (% h) of 75-90 or 50-95, optionally 65-95;
average RO₀₋₁₂ (% h) of 80-95; and
average RO₁₂₋₂₄ (% h) of 70-90.
   In particular embodiments of these methods, the method comprises providing an antagonist disclosed herein, optionally Compound A or Compound A, orally, at a dose of about 150 mg twice a day or about 450 mg twice a day.

In particular embodiments of the methods disclosed herein, the subject is provided with a dose or amount of the α4β7 integrin antagonist (or other agent) that does not saturate blood receptors, e.g., α4β7 integrin receptors, on circulating T cells. Thus, the dose or amount is one that results in sub-saturated blood receptor occupancy (% RO). In particular embodiments, the dose results in a % RO of less than 90%, less than 80%, less than 70%, less than 60%, less than 50%, less than 40%, less than 30%, less than 20%, or less than 10%. In certain embodiments, the % RO is less than 50% or less than 40%. % RO may be measured at drug levels or at maximum % RO. In certain embodiments, maximum RO is measured at about four hours post dose, whereas trough levels occur at about 24 hours post-dose. In certain embodiments, the method is practiced using a peptide dimer compound disclosed herein, e.g., Compound A or Compound B. In particular embodiments, the dose is provided orally or locally, e.g., rectally. In particular embodiments, the subject is provided with this dose once or twice a day.

In certain embodiments of the methods disclosed herein, the subject is provided with a dose or amount of the α4β7 integrin antagonist (or other agent) that achieves high antagonist levels and/or occupancy of T cell α4β7 in the gastrointestinal tissue. In particular embodiments, the dose results in occupancy of T cell α4β7 in the GI mucosa of at least 95%, at least 90%, at least 80%, at least 70%, at least 60%, at least 50%, at least 40%, or at least 30%. In certain embodiments, the method is practiced using a peptide dimer compound disclosed herein, e.g., Compound A or Compound B. In particular embodiments, the dose is provided orally or locally, e.g., rectally. In particular embodiments, the subject is provided with this dose once or twice a day.

In certain embodiments of the methods disclosed herein, the subject is provided with a dose or amount of the α4β7 integrin antagonist (or other agent) that achieves a ratio of % RO in the blood/% RO in Peyer's Patches (or other GI tissue) of less than 1.0, less than 0.9, less than 0.8, less than 0.7, less than 0.6, or less than 0.5.

In particular embodiments, the subject is provided with a dose or amount of about any of 5, 6, 7, 8, 9, 10, 12.5, 25.0, 37.5, 50.0, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, or 500.0 mg. In some embodiments, the subject is provided with a dose of about any of 12.5, 25.0, 37.5, 50.0, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 350.0, 400.0, 450.0, or 500.0 mg. In some embodiments, the subject is provided with a dose of about any of 70, 75, 80, 85, 90, 95, 100, 105, 110, 115, 120, 125, or 130 mg. In some embodiments, the subject is provided with a dose of about any of 85, 90, 95, 100, 105, 110, or 115 mg. In some embodiments, the subject is provided with a dose of about any of 95, 100, or 105 mg. In some embodiments, the subject is provided with a dose of about 100 mg. In some embodiments, the subject is provided with a dose ranging from about 100 mg to about 500 mg, optionally once daily or twice daily. In some embodiments, the subject is provided with a dose ranging from about 200 mg to about 1000 mg, optionally taken as a once daily dose or as divided doses (e.g., half the amount) twice daily. In some embodiments, the subject is provided with a dose ranging from about 100 mg to about 1500 mg per day, optionally taken as a once daily dose or as divided doses (e.g., half the amount) twice daily. In some embodiments, the subject is provided with a dose ranging from about 100 mg to about 1500 mg, once daily or twice daily. In some embodiments, the subject is provided with a dose of about any of 100, 150, 200, 250, 300, 250, 400, 450, or 500 mg once or twice daily. In some embodiments, the subject is provided with a dose of about any of 200, 300, 400, 500, 600, 700, 800, 900, or 1000 mg daily, optionally taken as a once daily dose or as divided doses (e.g., half the amount) twice daily. In some embodiments is provided with about 450 mg or about 150 mg, optionally twice a day. In particular embodiments, the subject is provided with anyboutt of these doses twice a day, optionally orally. In particular embodiments, the subject is provided with this dose once or twice a day. In some embodiments, this dose is divided and half is administered twice a day. In certain embodiments, the dose comprises a peptide dimer compound disclosed herein, e.g., Compound A or Compound B. In particular embodiments, the dose is provided orally or locally, e.g., rectally, optionally to treat an IBD, such as ulcerative colitis.

In particular embodiments of any of the methods disclosed herein, the subject is provided with a dose or amount of about any of 5, 6, 7, 8, 9, 10, 12.5, 25.0, 37.5, 50.0, 62.5, 75, 87.5, or 100.0 mg, optionally twice a day. In some embodiments, the subject is provided with a dose of about 6, 7, 8, 9, 10, 12.5, 25.0, or 37.5 mg. In some embodiments, the subject is provided with a dose ranging from about 5 mg to about 130 mg. In some embodiments, the subject is provided with a dose ranging from about 5 mg to about 50 mg. In some embodiments, the subject is provided with a dose ranging from about 5 mg to about 12.5 mg. In some embodiments, the subject is provided with a dose of about 8 mg. in some embodiments, the subject is provided with a dose of about 150 mg twice daily or a dose of about 450 mg twice daily. In particular embodiments, the subject is provided with any of these doses twice a day, optionally orally. In particular embodiments, the subject is provided with any of these doses once or twice a day. In particular embodiments, it is provided twice a day. In some embodiments, the subject is provided with a dose of about 8 mg. In some embodiments, the subject is provided with a dose of about 150 mg twice daily or a dose of about 450 mg twice daily. In certain embodiments, the dose comprises a peptide dimer compound disclosed herein, e.g., Compound A or Compound B. In particular embodiments, the dose is provided orally or locally, e.g., rectally, e.g., by suppository. In some embodiments, the subject is provided orally with a dose of about 150 mg twice daily or a dose of about 450 mg twice daily of Compound A or Compound B twice daily, optionally to treat an IBD, such as ulcerative colitis.

In certain embodiment of methods disclosed herein, the method is for treating an individual or subject afflicted with an inflammatory disease or disorder. In particular embodiments, the condition is an inflammatory condition of the gastrointestinal system. In certain embodiments, the subject is administered or provided with a dose or amount of an α4β7 integrin antagonist that results in sub-saturated blood receptor occupancy (% RO). In certain embodiments, the method is practiced using a peptide dimer compound disclosed herein, e.g., Compound A or Compound B. In particular embodiments, the dose is provided orally or locally, e.g., rectally. In particular embodiments, the subject is provided with this dose once or twice a day.

In certain embodiments, the disease or disorder is selected from the group consisting of: Inflammatory Bowel Disease (IBD), adult IBD, pediatric IBD, adolescent IBD, ulcerative colitis, Crohn's disease, Celiac disease (nontropical Sprue), enteropathy associated with seronegative arthropathies, microscopic colitis, collagenous colitis, eosinophilic gastroenteritis, radiotherapy, chemotherapy, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, primary sclerosing cholangitis, human immunodeficiency virus (HIV) infection in the GI tract, eosinophilic asthma, eosinophilic esophagitis, gastritis, colitis, microscopic colitis, and graft versus host disease (GVDH). In particular embodiments, the disease or disorder is an IBD. In some embodiments, the IBD is ulcerative colitis. In some embodiments, the IBD is Crohn's disease. In some embodiments, the subject is provided Compound A or Compound B orally to treat ulcerative colitis or Crohn's disease.

In certain embodiments, the disclosure provides a method of treating an IBD in a subject in need thereof, comprising orally administering to the subject a peptide dimer compound disclosed herein, e.g., Compound A or Compound B, wherein the compound is administered at a dosage that results in sub-saturating blood receptor occupancy, e.g., less than 50% RO. In certain embodiments, the IBD is ulcerative colitis or Crohn's disease. In particular embodiments, the subject is provided with a dose or amount of about any of 5, 6, 7, 8, 9, 10, 12.5, 25.0, 37.5, 50.0, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, or 500.0 mg. In some embodiments, the subject is provided with a dose of about any of 12.5, 25.0, 37.5, 50.0, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 350.0, 400.0, 450.0, or 500.0 mg. In some embodiments, the subject is provided with a dose of about any of 70, 75, 80, 85, 90, 95, 100, 105, 110, 115, 120, 125, or 130 mg. In some embodiments, the subject is provided with a dose of about any of 85, 90, 95, 100, 105, 110, or 115 mg. In some embodiments, the subject is provided with a dose of about any of 95, 100, or 105 mg. In some embodiments, the subject is provided with a dose of about 100 mg. In some embodiments, the subject is provided with a dose ranging from about 100 mg to about 500 mg, optionally once daily or twice daily. In some embodiments, the subject is provided with a dose ranging from about 200 mg to about 1000 mg, optionally taken as a once daily dose or as divided doses (e.g., half the amount) twice daily. In some embodiments, the subject is provided with a dose ranging from about 100 mg to about 1500 mg per day, optionally taken as a once daily dose or as divided doses (e.g., half the amount) twice daily. In some embodiments, the subject is provided with a dose ranging from about 100 mg to about 1500 mg, once daily or twice daily. In some embodiments, the subject is provided with a dose of about any of 100, 150, 200, 250, 300, 250, 400, 450, or 500 mg once or twice daily. In some embodiments, the subject is provided with a dose of about any of 200, 300, 400, 500, 600, 700, 800, 900, or 1000 mg daily, optionally taken as a once daily dose or as divided doses (e.g., half the amount) twice daily. In some embodiments is provided with about 450 mg or about 150 mg, optionally twice a day. In some embodiments, the subject is provided a dose of about 150 mg twice daily or a dose of about 450 mg twice daily of Compound A or Compound B orally to treat ulcerative colitis (UC) or Crohn's disease. In certain embodiments, the method is used to treat a subject for ulcerative colitis. In particular embodiments, the subject has moderate to severe active UC. IN certain embodiments, subjects have a biopsy-confirmed diagnosis of UC. In certain embodiments, the subject meets one or more (or all) of the inclusion criteria disclosed in the Examples, and does not meet one or more (or any) of the exclusion criteria disclosed in the Examples.

In certain embodiments, the disclosure provides a method of treating an IBD (e.g., ulcerative colitis or Crohn's disease) in a subject in need thereof, comprising orally administering to the subject a peptide dimer compound disclosed herein, e.g., Compound A or Compound B, wherein the compound is administered at a dosage that results in one or more of the following pharmacokinetic parameters being met in plasma of the subject:
Cmax (ng/mL) of 1-25, optionally 4-12;
Tmax (h) of 1-5, optionally 2-4;
AUCₜ (ng·h/mL) of 10-250, optionally 50-150;
AUC_{inf} (ng·h/mL) of 10-300, optionally 30-250;
t_{1/2} (h) of 3-10, optionally 4-10;
AUCₜₐᵤ (ng·h/mL) of 30-130;
Ctrough (ng/mL) of 1-5;
accumulation Cmax (ng·mL) of 0.5-2.5, optionally 2-3; and
accumulation AUCₜ (ng·h/mL) of 0.5-3.0.
   In particular embodiments of these methods, the IBD is ulcerative colitis or Crohn's disease. In particular embodiments, the pharmacokinetic parameter is met within 1 hour, within 2 hours, within 3 hours, within 4 hours, within 6 hours, within 8 hours, or within 12 hours of administration. In particular embodiments, the pharmacokinetic parameter is maintained for at least 1 hour, at least 2 hours, at least 3 hours, at least 4 hours, at least 6 hours, at least 8 hours, or at least 12 hours following administration.

In certain embodiments, the disclosure provides a method of treating an IBD in a subject in need thereof, comprising orally administering to the subject a peptide dimer compound disclosed herein, e.g., Compound A or Compound B, wherein the compound is administered at a dosage that results in one or more of the following pharmacodynamic parameters in plasma of the subject:
ROmax (%) of 50-100, optionally 90-100;
average RO (%) of 50-95, optionally 65-95;
change in receptor expressionₘₐₓ (%) of −20 to −60, optionally −35 to −60;
average change in receptor expression (%) of −10 to −55, optionally −25 to −55;
steady state ROmax (%) of 80-100;
average RO₀₋₂₄ (% h) of 75-90;
average RO₀₋₁₂ (% h) of 80-95; and
average RO₁₂₋₂₄ (% h) of 70-90.

In particular embodiments of these methods, the IBD is ulcerative colitis or Crohn's disease. In particular embodiments, the pharmacodynamic parameter is met within 1 hour, within 2 hours, within 3 hours, within 4 hours, within 6 hours, within 8 hours, or within 12 hours of administration. In particular embodiments, the pharmacodynamic parameter is maintained for at least 1 hour, at least 2 hours, at least 3 hours, at least 4 hours, at least 6 hours, at least 8 hours, or at least 12 hours following administration.

In certain embodiments, methods disclosed herein reduce the activity (partially or fully) of α4β7 in the subject. In certain embodiments, the methods reduce the proliferation of T cells comprising the α4β7 integrin, for example, the proliferation of T cells present in gastrointestinal tissue, e.g., gastrointestinal mucosa, of the subject. In further embodiments, the methods inhibit the generation or release of cytokines by T cells in the subject, e.g., T cells in gastrointestinal tissue of the subject, e.g., β7+ T cells. In particular embodiments, the methods reduce the generation or release of any of the cytokines disclosed in the accompanying figures, e.g., IFNgamma, interleukin-6 (IL-6), IL-8, IL-12/23p40, IL-15, IL-16, IL-13, vascular endothelial growth factor (VEGF), granulocyte-macrophage colony-stimulating factor (GM-CSF), tumor necrosis factor alpha (TNFalpha), or tumor necrosis factor beta (TNFbeta). In certain embodiments, the methods disclosed herein inhibit the generation or release of cytokines by T cells, whose release is promoted by binding to mucosal vascular addressin cell adhesion molecule 1 (MAdCAMI1), in the subject, e.g., in gastrointestinal tissue, such as gastrointestinal mucosa. In particular embodiments, the T cells are CD45RO− naïve or CD45RO+ memory T-cells. In certain embodiments, the T cells are β7⁺.

In a further related embodiments, the present invention includes a method for treating a subject, e.g., a mammal or human, afflicted with a condition that is associated with a biological function α4β7, comprising providing or administering to the subject a peptide molecule described herein in an amount sufficient to inhibit (partially or fully) the biological function of α4β7 in tissues expressing MAdCAMI1, e.g., gastrointestinal tissue, such as the gastrointestinal mucosa. In particular embodiments, the subject is provided with an effective amount of the peptide monomer or peptide dimer sufficient to at least partially inhibit the biological function of α4β7 in a tissue expressing MAdCAM1. In certain embodiments, the condition is inflammatory bowel disease.

In additional embodiments, the invention includes a method of treating or preventing a disease or condition in a subject in need thereof, comprising providing or administering to the subject, e.g., a mammal, an effective amount of a peptide dimer or peptide monomer described herein, wherein the disease or condition is selected from the group consisting of Inflammatory Bowel Disease (IBD) (including adult IBD, pediatric IBD and adolescent IBD), ulcerative colitis, Crohn's disease, Celiac disease (nontropicalSprue), enteropathy associated with seronegative arthropathies, microscopic colitis, collagenous colitis, eosinophilic gastroenteritis, radiotherapy, chemotherapy, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, primary sclerosing cholangitis, human immunodeficiency virus (HIV) infection in the GI tract, eosinophilic asthma, eosinophilic esophagitis, gastritis, colitis, microscopic colitis and graft versus host disease (GVDH) (including intestinal GVDH). In particular embodiments of any of the methods of treatment described herein, the subject has been diagnosed with or is considered to be at risk of developing one of these diseases or conditions.

In particular embodiments of any of the methods of treatment described herein, the peptide molecule (or pharmaceutical composition comprising the peptide molecule) is administered to the individual by a form of administration selected from the group consisting of oral, intravenous, peritoneal, intradermal, subcutaneous, intramuscular, intrathecal, inhalation, vaporization, nebulization, sublingual, buccal, parenteral, rectal, vaginal, and topical.

In particular embodiments, the disclosure provides a unit dosage form of a peptide dimer compound disclosed herein, comprising about any of 5, 6, 7, 8, 9, 10, 12.5, 25.0, 37.5, 50.0, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, or 500.0 mg. In some embodiments, the unit dosage form comprises about any of 12.5, 25.0, 37.5, 50.0, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 350.0, 400.0, 450.0, or 500.0 mg. In some embodiments, the unit dosage form comprises about any of 70, 75, 80, 85, 90, 95, 100, 105, 110, 115, 120, 125, or 130 mg. In some embodiments, the unit dosage form comprises about any of 85, 90, 95, 100, 105, 110, or 115 mg. In some embodiments, the unit dosage form comprises about any of 95, 100, or 105 mg. In some embodiments, the unit dosage form comprises about 100 mg. In some embodiments, the unit dosage form comprises from about 100 to 500 mg. In some embodiments, the unit dosage form comprises about any of 100, 150, 200, 250, 300, 250, 400, 450, or 500 mg. In some embodiments, unit dosage form comprises about 450 mg or about 150 mg. In particular embodiments, the unit dosage form comprises a pharmaceutical composition comprising the peptide dimer compound, e.g., any of those disclosed herein. In particular embodiments, it is formulated for oral administration, e.g., as a tablet. In certain embodiments, it is formulated for rectal administration, e.g., as a suppository. In some embodiments, the unit dosage form comprises about 450 mg or about 150 mg of Compound A or Compound B (or a pharmaceutically acceptable salt thereof). In particular embodiments, the unit dosage form comprises a pharmaceutical composition comprising the peptide dimer compound, e.g., any of those disclosed herein.

In particular embodiments, the peptide molecules of the present invention are present in a pharmaceutical composition further comprising one or more pharmaceutically acceptable diluents, carriers, or excipients. In particular embodiments, they are formulated as a liquid or solid. In particular embodiments, they are formulated as a tablet or capsule, or as a liquid suspension. Some embodiments of the present invention further provide a method for treating an individual with an α4β7 integrin antagonist peptide molecule of the present invention that is suspended in a sustained-release matrix. A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. On particular biodegradable matrix is a matrix of one of either polylactide, polyglycolide, or polylactide co-glycolide (co-polymers of lactic acid and glycolic acid).

In some aspects, the invention provides a pharmaceutical composition for oral delivery. The various embodiments and peptide molecule compositions of the instant invention may be prepared for oral administration according to any of the methods, techniques, and/or delivery vehicles described herein. Further, one having skill in the art will appreciate that the peptide molecule compositions of the instant invention may be modified or integrated into a system or delivery vehicle that is not disclosed herein, yet is well known in the art and compatible for use in oral delivery of small peptide molecules.

Oral dosage forms or unit doses compatible for use with the peptides of the present invention may include a mixture of peptide active drug components, and nondrug components or excipients, as well as other non-reusable materials that may be considered either as an ingredient or packaging. Oral compositions may include at least one of a liquid, a solid, and a semi-solid dosage forms. In some embodiments, an oral dosage form is provided comprising an effective amount of a peptide molecule described herein, wherein the dosage form comprises at least one of a pill, a tablet, a capsule, a gel, a paste, a drink, and a syrup. In some instances, an oral dosage form is provided that is designed and configured to achieve delayed release of the thioether peptide molecule in the small intestine of the subject.

In one embodiment, an oral pharmaceutical composition comprising a peptide of the present invention comprises an enteric coating that is designed to delay release of the peptide molecule in the small intestine. In some instances it is preferred that a pharmaceutical composition of the instant invention comprise an enteric coat that is soluble in gastric juice at a pH of about 5.0 or higher. In at least one embodiment, a pharmaceutical composition is provided comprising an enteric coating comprising a polymer having dissociable carboxylic groups, such as derivatives of cellulose, including hydroxypropylmethyl cellulose phthalate, cellulose acetate phthalate and cellulose acetate trimellitate and similar derivatives of cellulose and other carbohydrate polymers.

In one embodiment, a pharmaceutical composition comprising a peptide molecule described herein is provided in an enteric coating, the enteric coating being designed to protect and release the pharmaceutical composition in a controlled manner within the lower gastrointestinal system of a subject, and to avoid systemic side effects. In addition to enteric coatings, the peptide molecules of the instant invention may be encapsulated, coated, engaged or otherwise associated within any compatible oral drug delivery system or component. For example, in some embodiments a peptide molecule of the present invention is provided in a lipid carrier system comprising at least one of polymeric hydrogels, nanoparticles, microspheres, micelles, and other lipid systems.

To overcome peptide degradation in the small intestine, some implementations of the present invention comprise a hydrogel polymer carrier system in which a peptide molecule in accordance with the present invention is contained, whereby the hydrogel polymer protect the peptide from proteolysis in the small intestine. The peptide molecules of the present invention may further be formulated for compatible use with a carrier system that is designed to increase the dissolution kinetics and enhance intestinal absorption of the peptides. These methods include the use of liposomes, micelles and nanoparticles to increase GI tract permeation of peptides.

Various bioresponsive systems may also be combined with one or more thioether peptide molecules of the present invention to provide a pharmaceutical agent for oral delivery. In some embodiments, a peptide molecule of the instant invention is used in combination with a bioresponsive system, such as hydrogels and mucoadhesive polymers with hydrogen bonding groups (e.g., PEG, poly(methacrylic) acid [PMAA], cellulose, Eudragit®, chitosan and alginate) to provide a therapeutic agent for oral administration. Other embodiments include a method for optimizing or prolonging drug residence time for a peptide molecule disclosed herein, wherein the surface of the peptide molecule is modified to comprise mucoadhesive properties through hydrogen bonds, polymers with linked mucins or/and hydrophobic interactions. These modified peptide molecules may demonstrate increase drug residence time within the subject, in accordance with a desired feature of the invention. Moreover, targeted mucoadhesive systems may specifically bind to receptors at the enterocytes and M-cell surfaces, thereby further increasing the uptake of particles containing the peptide molecules.

Other embodiments comprise a method for oral delivery of a peptide molecule described herein wherein the peptide molecule is used in combination with permeation enhancers that promote the transport of the peptides across the intestinal mucosa by increasing paracellular or transcellular permeation. For example, in one embodiment a permeation enhancer is combined with a peptide molecule described herein, wherein the permeation enhancer comprises at least one of a long-chain fatty acid, a bile salt, an amphiphilic surfactant, and a chelating agent. In one embodiment, a permeation enhancer comprising sodium N-[(hydroxybenzoyl)amino] caprylate is used to form a weak noncovalent association with the peptide molecule of the instant invention, wherein the permeation enhancer favors membrane transport and further dissociation once reaching the blood circulation. In another embodiment, a peptide molecule is conjugated to oligoarginine, thereby increasing cellular penetration of the peptide into various cell types. Further, in at least one embodiment a noncovalent bond is provided between a peptide molecule described herein and a permeation enhancer selected from the group consisting of a cyclodextrin (CD) and a dendrimers, wherein the permeation enhancer reduces peptide aggregation and increasing stability and solubility for the peptide molecule.

When used in at least one of the treatments or delivery systems described herein, a therapeutically effective amount of one of the peptide molecules of the present invention may be employed in pure form or, where such forms exist, in pharmaceutically acceptable salt form. As used herein, a “therapeutically effective amount” of the compound of the invention is meant to describe a sufficient amount of the peptide molecule to treat an integrin-related disease, (for example, to reduce inflammation associated with IBD) at a desired benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific compound employed; e) the duration of the treatment; f) drugs used in combination or coincidental with the specific compound employed, and like factors well known in the medical arts.

Alternatively, a compound of the present invention may be administered as pharmaceutical compositions containing the peptide molecule of interest in combination with one or more pharmaceutically acceptable excipients. A pharmaceutically acceptable carrier or excipient refers to a non-toxic solid, semi-solid or liquid filler, diluent, encapsulating material or formulation auxiliary of any type. The compositions may be administered parenterally, intracisternally, intravaginally, intraperitoneally, intrarectally, topically (as by powders, ointments, drops, suppository, or transdermal patch), rectally, or buccally. The term “parenteral” as used herein refers to modes of administration which include intravenous, intramuscular, intraperitoneal, intrasternal, subcutaneous, intradermal and intraarticular injection and infusion.

Compositions for rectal or vaginal administration are preferably suppositories which may be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Total daily dose of the compositions of the invention to be administered to a human or other mammal host in single or divided doses may be in amounts, for example, from 0.0001 to 300 mg/kg body weight daily and more usually 1 to 300 mg/kg body weight.

### EXAMPLES

### Example 1

### Compound A Blocks MAdCAM1-Mediated CD4+ T Cell Proliferation

Compound A, an oral gastrointestinal (GI)-restricted peptide antagonist of α4β7 integrin, is being developed for the treatment of Inflammatory Bowel Disease (IBD). Blockade of α4β7 binding to mucosal addressin cell adhesion molecule-1 (MAdCAM1) is thought to treat IBD by preventing extravascular migration of blood T-cells into the inflamed GI mucosa. The following experiment was conducted to further explore the mechanism by which Compound A reduces GI inflammation. In particular, the potential of a local GI-acting function of α4β7 was assessed by evaluating the ability of Compound A to inhibit MAdCAM1-mediated CD4⁺ T-cell proliferation and cytokine production.

### Compound A:

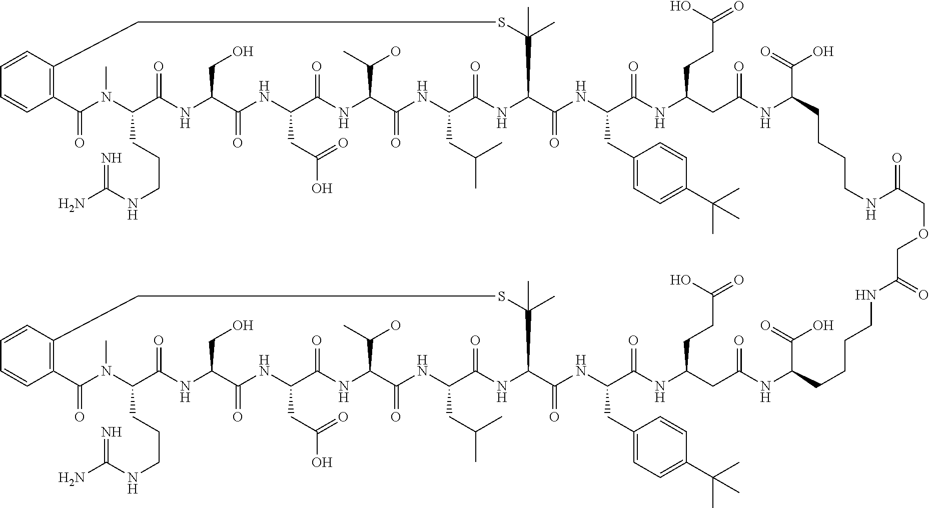 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

((2-Benzyl)-(N-Me-R)-Ser-Asp-Thr-Leu-Pen-(Phe(4-tBlu))-(β-homo-Glu)-(D-Lys)-OH)₂ (SEQ ID NO: 5) and linker-DIG diglycolic acid.

PBMCs were purified from healthy human donors and enrichment of CD4+ T cells was performed. Primary CD4⁺ T-cells were labeled fluorescently and incubated with plate bound anti-CD3 alone or together with MAdCAM1 with or without inhibitors (or negative control): Compound A (1 uM), an inactive analog as negative control (1 uM), or vedolizumab (500 ng/mL) for three days. Phenotype, distribution of T-helper (Th) subsets and % RO analyses were conducted by flow cytometry of freshly stained live samples.

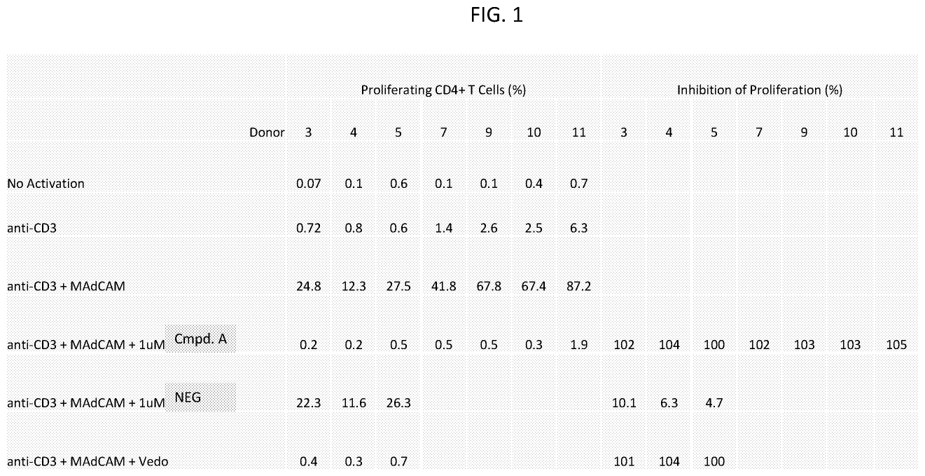

MAdCAM1 combined with anti-CD3 markedly enhanced proliferation of CD4⁺ T-cells compared to anti-CD3 alone (n=7, 12-87%) (FIG. 1) after 3 days of incubation. Compound A completely abolished MAdCAM1-mediated proliferation (FIG. 1). The level of inhibition was similar to inhibition by vedolizumab (FIG. 1). Blockade was not observed with the inactive analog (negative control; NEG) indicating dependency on Compound A binding to α4β7. Inhibition by Compound A was dependent on the activity of Compound A. Inhibition by Compound A was concentration dependent. The mean IC₅₀ from four independent human donors was 4.4 nM (Table 4).

TABLE 4
IC50 from Four Donors

| N = 3 Donor | IC50 (nM) |
|---|---|
| Donor 7 | 1.6 |
| Donor 8 | 3.7 |
| Donor 9 | 5.2 |
| Donor 11 | 8.0 |
| Average | 3.5 |
| Standard Deviation | 1.8 |

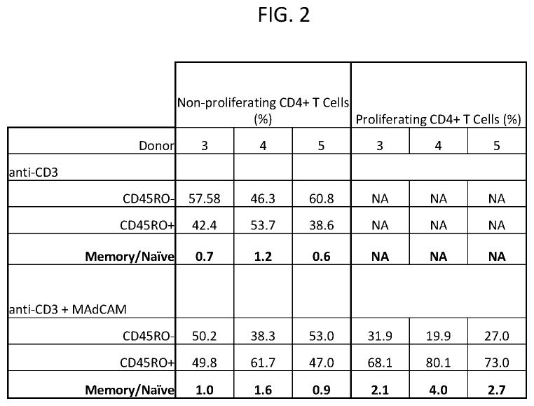

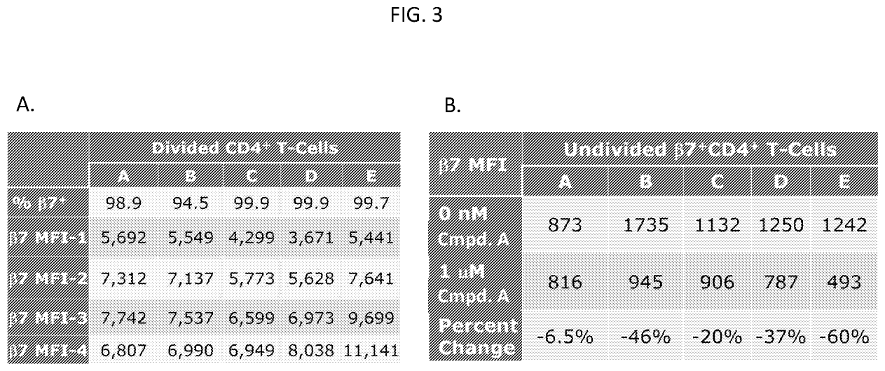

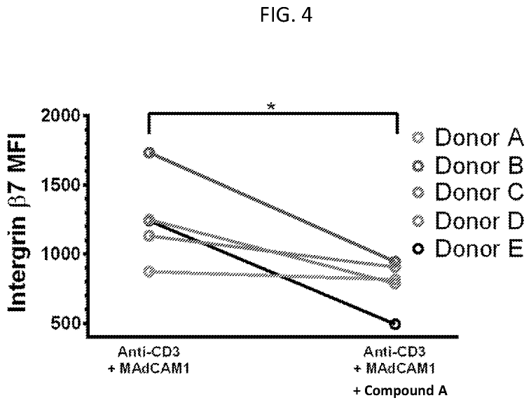

Immune phenotyping revealed that proliferation occurred in both CD45RO⁻ naïve and CD45RO⁺ memory T-cells and shifted naïve T cells toward a memory cell phenotype (FIG. 2). The proliferation was restricted to the β7⁺ population with successive cycles of proliferation showing increased β7 expression (FIG. 3A). Surface expression of β7 was lowered in undivided CD4⁺ T-cells in the presence of Compound A, indicative of internalization by Compound A (FIG. 3B and FIG. 4; tested in 5 donors). Amongst the proliferated memory T-cells, the percentage of the IFNγ-producing Th1 subset was higher than IL-17A-producing Th17 and IL-4-producing Th2 subsets (Table 5).

TABLE 5
Characteristics of Proliferated CD4+ T-cells

| | CD4+ T-cells | | |
|---|---|---|---|
| Human Donor ID | Th1-IFNγ | Th2-IL-4 | Th17-IL-17A |
| No activation | 7.4% | 0.3% | 0.5% |
| Anti-CD3 | 47% | 2.2% | 2.8% |
| Anti-CD3 + MAdCAM1 | 15% | 0.6% | 1.1% |
| Anti-CD3 + MAdCAM1 + | 15% | 0.7% | 0.6% |
| 1 uM Compound A | | | |

The α4β7-MAdCAM1 interaction promoted β7⁺CD4⁺ T-cell proliferation and cytokine release, which may contribute to chronic inflammatory responses occurring in the diseased gut of IBD patients independent of T-cell trafficking. Compound A inhibition of MAdCAM1-mediated signaling through α4β7 supports the potential therapeutic advantages for an oral GI-restricted approach, whereby Compound A is delivered locally and directly blocks α4β7 function in the GI.

### Example 2

### Compound A Blocks MAdCAM1-Mediated Cytokine Production

The following experiment was conducted to further explore the mechanism by which Compound A reduces GI inflammation. In particular, cytokine profiling was conducted on T cells isolated from normal, healthy donors.

PBMCs were purified from three healthy human donors (Donors 7, 10, and 11), and enrichment of CD4+ T cells was performed. Primary CD4⁺ T-cells were labeled fluorescently and incubated with plate bound anti-CD3 alone, plate bound anti-CD3 together with MAdCAM1, or plate bound anti-CD3 together with MAdCAM1 and various amounts of Compound A. Supernatant cytokine levels were quantified by MSD or luminex platform multiplex assays for anti-CD3 alone, and anti-CD3+MAdCAM1 in the presence of varying concentrations of Compound A.

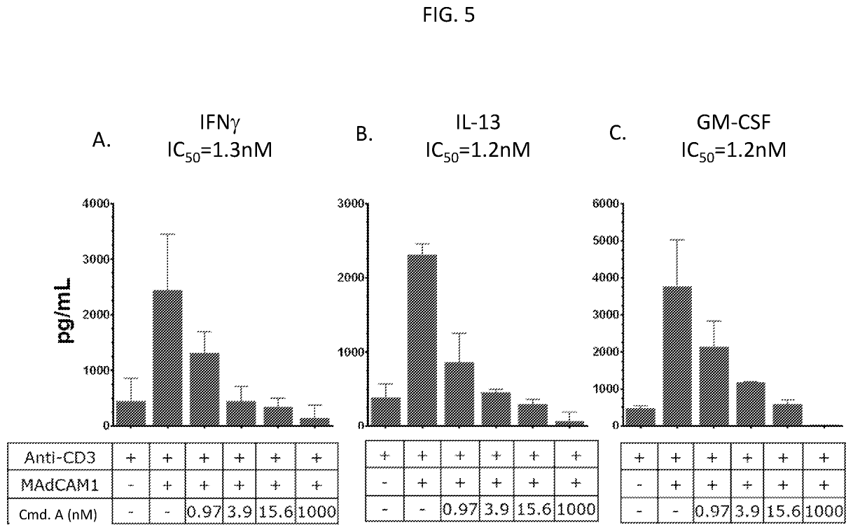

Multiplex profiling identified several cytokines, including IFNγ, IL-5, IL-6, IL-10, IL-13, GM-CSF and TNFα, whose release were promoted by MAdCAM1 (FIGS. 5A-C and 6A-C). MAdCAM1-mediated cytokine production was inhibited by Compound A in a concentration-dependent manner. Concentration-dependent and full inhibition of MAdCAM1-mediated production of specific cytokines by Compound A is shown in FIGS. 5A-C and 6A-C. The α4β7-MAdCAM1 interaction promoted β7⁺CD4⁺ T-cell proliferation and cytokine release, which may contribute to chronic inflammatory responses occurring in the diseased gut of IBD patients independent of T-cell trafficking. Compound A inhibition of MAdCAM1-mediated signaling through α4β7 supports the therapeutic advantages for an oral GI-restricted approach, whereby Compound A is delivered locally and directly blocks α4β7 function in the GI.

### Example 3

### Receptor Occupancy in Mice

The following experiment was conducted to examine receptor occupancy in whole blood and Peyer's patches in mice dosed with the Compound A analog, Compound B.

### Compound B:

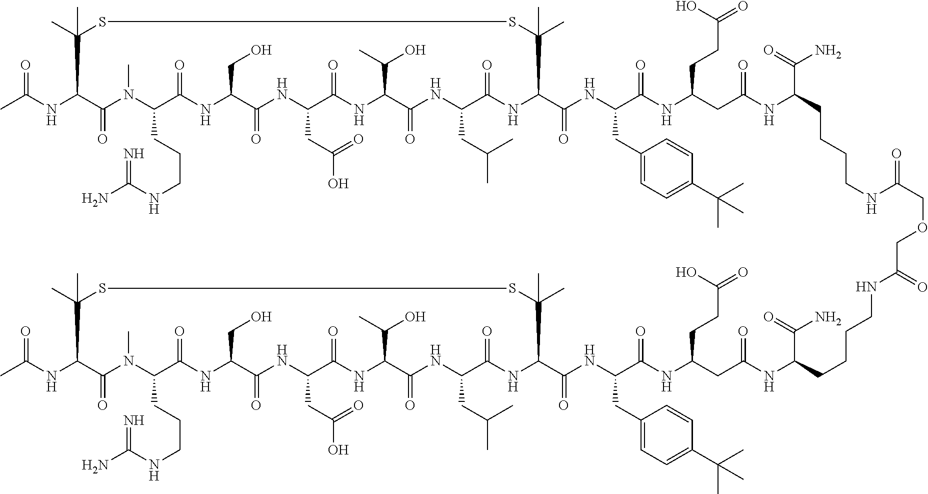 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)[C@@H](NC(C)=O)C(C)(C)SSC2(C)C)C(N)=O)C(N)=O)C(C)(C)SSC1(C)C] [0267] (Ac-Pen-(N-Me-Arg)-Ser-Asp-Thr-Leu-Pen-(Phe(4-tBu))-(β-homo-Glu)-(D-Lys)-NH₂)₂ (SEQ ID NO. 6) with linker=DIG (diglycolic acid)

Three groups of female, C57BL/6 mice (N=6 per group) were treated orally with either vehicle (Group 1), Compound B (3 mg/kg, P0, QD; Group 2), or Compound B (30 mg/kg, PO, QD; Group 3). One hour following dosing, mice were euthanized and whole blood/plasma and Peyer's patches were collected. Peyer's patches were dispersed in 1 mL RPMI medium with 2% FBS without washing step. Whole blood and single cell suspension of Peyer's patches (100 uL out of 1 mL total) were submitted for flow cytometry to determine α4β7 receptor occupancy. % RO=(1−(% positive test sample/% positive median vehicle control))*100. Plasma and Peyer's patches dispersed in single cell suspension (500 uL of 1 mL total) were also analyzed for drug exposure.

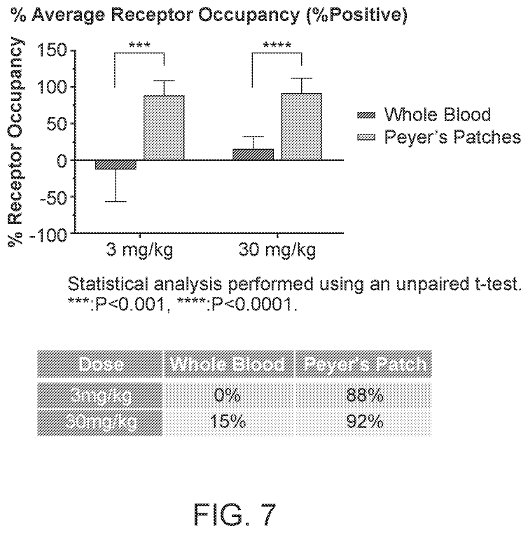

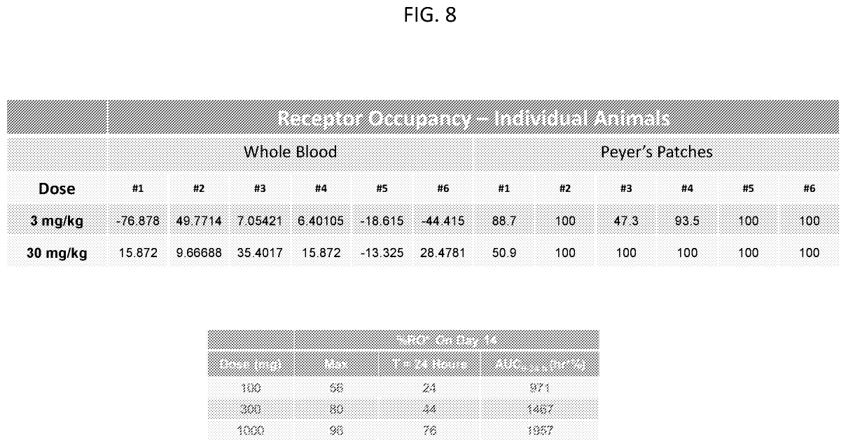

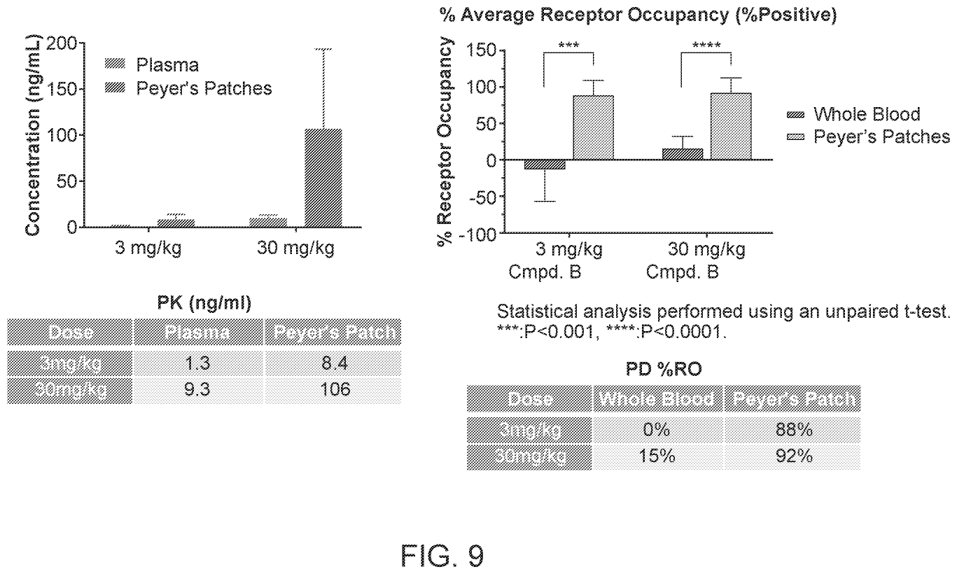

There was a significantly higher receptor occupancy in Peyer's patches compared to whole blood in both dose groups (FIG. 7). The levels of receptor occupancy in whole blood or Peyer's patches between the dose groups were comparable. There was compete (100%) receptor occupancy in Peyer's patches in some animals in both the 3 and 30 mg/kg doses (FIG. 8, top). % RO for various doses of Compound B is shown in FIG. 8, bottom. PK and PD data is shown in FIG. 9 with Compound B doses shown.

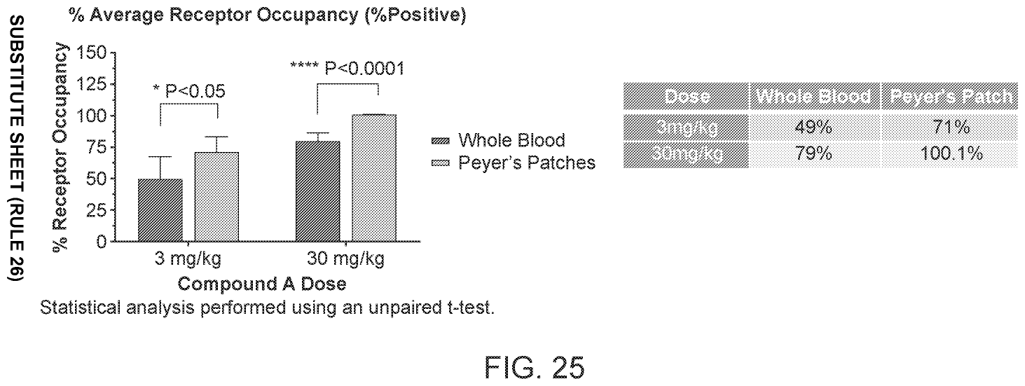

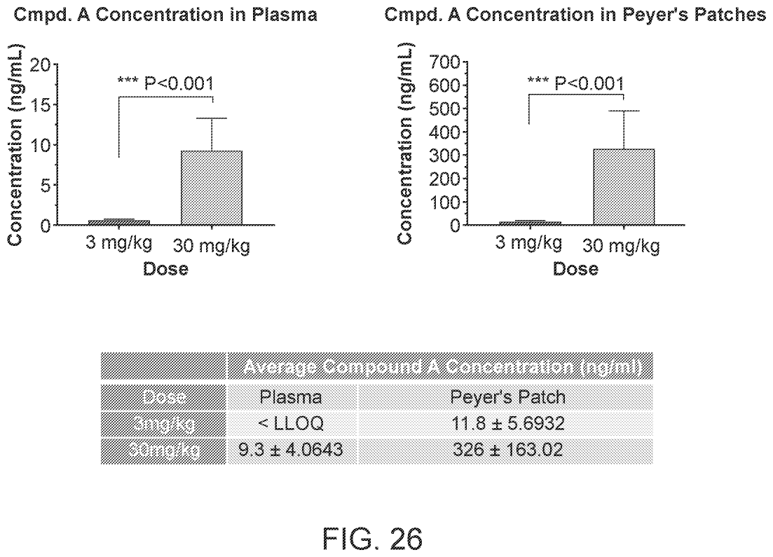

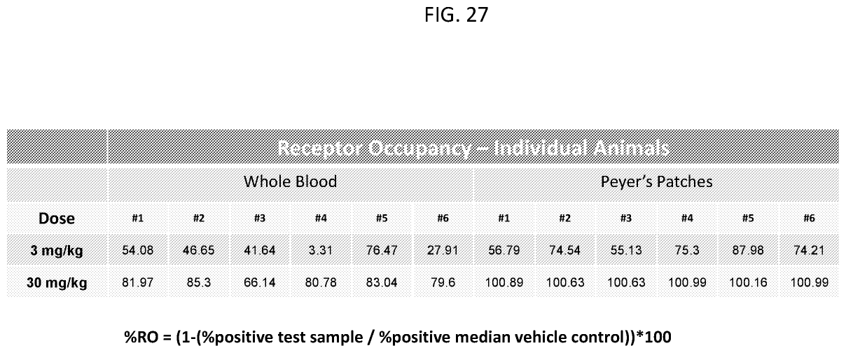

A similar experiment was performed using Compound A. There was a significantly higher receptor occupancy in Peyer's Patches compared to whole blood in both dose groups. There was a significantly higher receptor occupancy in whole blood (P<0.01) and Peyer's Patches (P<0.001) at the 30 mg/kg dose when compared to the 3 mg/kg dose (FIG. 25). The effect was the same observed with Compound B, although quantitatively not as great, potentially since Compound A has greater activity than Compound B, but the same doses were used. There was also a significant dose-dependent increase in Compound A concentration in both plasma and Peyer's Patches (FIG. 26). Compound A concentration was significantly higher in Peyer's Patches than plasma at both dose levels. As shown in FIG. 27, there was a dose dependent increase in receptor occupancy in the whole blood and Peyer's Patches of animals dosed with Compound A. There was complete (100%) receptor occupancy in Peyer's Patches in animals at the 30 mg/kg dose.

### Example 4

### Compound A Tissue Exposure in Mice

This study was performed to determine plasma, Peyer's patch (PP), and mesenteric lymph nodes (MLN), small intestine, and colon tissue exposure of Compound A after PO administration in healthy C57BL/6 female mice.

Twelve treatment-naïve C57BL/6 female mice were assigned to the study. Animals were fasted overnight and administered with a single dose of 30 mg/kg Compound A by oral gavage (PO) at a dose volume of 10 mL/kg. At 1, 3, and 6 hours (h) post-dose, 4 mice/time point were subjected to terminal bleeds and euthanized; Peyer's patches (PPs), mesenteric lymph nodes (MLNs), small intestine, and colon were collected from each animal. The blood was processed to plasma; plasma and the tissue samples were submitted for pharmacokinetic (PK) analysis of Compound A levels using a qualified liquid chromatography tandem mass spectrometry (LC-MS/MS) method.

The concentrations of Compound A in plasma and tissues were analyzed using a qualified liquid chromatography tandem mass spectrometry (LC-MS/MS) method. Processed plasma and tissue samples were analyzed on an AB/MDS Sciex API 4000 mass spectrometer. Positive ions were monitored in the multiple reaction-monitoring (MRM) mode. Quantitation was by peak area ratio.

PK data analysis was carried out using non-compartmental analysis (NCA) in Phoenix WinNonlin 8.1 (Certara USA Inc.). All concentration values below the lower limit of quantitation were treated as zero for the pharmacokinetic analysis. The maximum concentration (Cₘₐₓ) and the apparent time to Cₘₐₓ (tₘₐₓ) were obtained by observation. The area under the concentration versus time curve (AUC) was obtained by the linear trapezoidal method. All concentration data and PK parameters were reported at up to three significant figures if the value was greater than one, and at up to three decimal places if the value was less than one. Time parameters were reported at up to two decimal places. The concentration data were plotted using Excel (Microsoft).

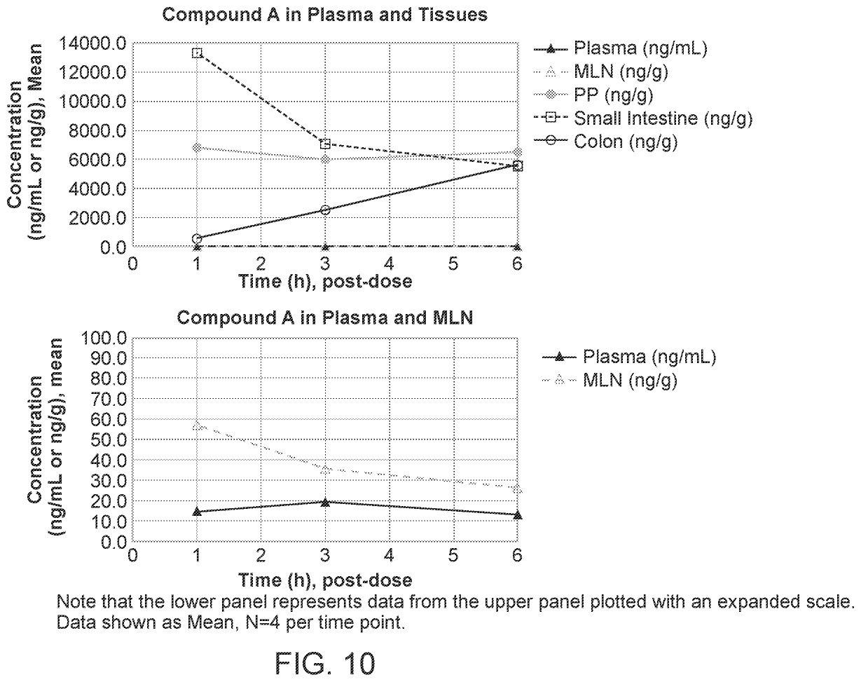

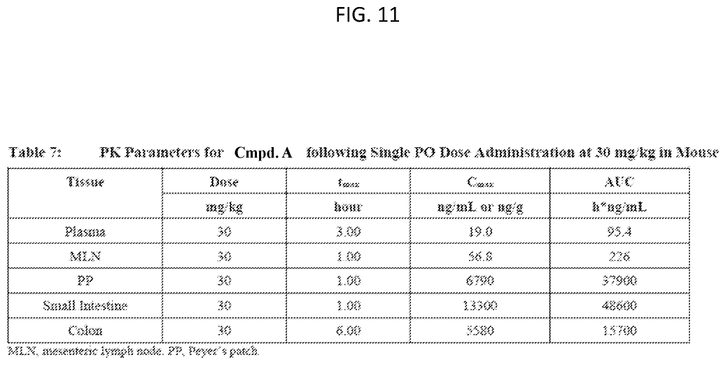

The mean values of plasma and tissue concentrations of Compound Ain each animals are plotted in FIG. 10. The resulting PK parameters are provided in FIG. 11. Following a single PO administration at 30 mg/kg, peak Compound A exposure was observed at 1 h in MLNs, PPs, and small intestine, at 3 h in plasma, and at 6 h in colon. The mean Cmax values were largest in the small intestine (13300 ng/g), with approximately two-fold lower mean values seen in PPs and colon. These gastrointestinal levels were much greater (by a factor of 100 or more) than those in plasma (19.0 ng/mL) and MLNs (56.8 ng/g). Similarly, mean AUC values in the small intestine, PPs, and colon (48600, 37900, and 15700 ng/g, respectively) were much larger (by a factor of 60 or more) than those in plasma (95.4 ng/mL) and MHLNs (226 ng/g). These results demonstrated that Compound A has limited plasma and lymph node exposure when dosed PO in otherwise healthy female mice. Dose analysis (data not shown) demonstrated that the dosing solutions was 97.6% of the nominal concentration.

### Example 5

### Compound A Inhibits Gut Homing of Cultured T-Cells

T cells cultured in the presence of all-trans-retinoic acid (ATRA) upregulate gut homing receptors CCR9, integrin α4 (α4) and integrin β7 (β7), and preferentially home to gut tissue (ileal lamina propria and Peyer's patches). The goal of this study was to analyze gut homing of T cells cultured in the presence of Compound A.

Purified CD3+ cells were isolated from B6.SJL (CD45.1+) donor mice and cultured in the presence of anti-CD3/anti-CD28 beads and IL-2 to induce T cell activation and proliferation. In some culture conditions, Compound A and/or ATRA were added. To track the cells in vivo, ATRA− and ATRA+ cells were labeled with CMFDA and CTFR respectively. These labeled cells were then co-injected into C57BL/6 (CD45.2+) recipient mice. There were 4 groups of recipient mice in the study:
Vehicle (negative control)
Anti-VLA-4 (treated in vivo with anti-VLA-4, the positive control)
Compound A, 100 nM (test, in culture)
Compound A, 1000 nM (test, in culture)

The proportion of ATRA− and ATRA+ cells in spleen, Peyer's patches (PP), and ileal lamina propria (LP) of the recipient mice was measured by flow cytometry to evaluate cell homing.

Cells cultured in the presence of ATRA+/DMSO (“ATRA+/DMSO cells”) had a higher proportion of cells expressing gut homing receptors CCR9 and integrins α4 and β7 than cell cultured in the absence of ATRA (“ATRA− cells”), as expected. ATRA+/Compound A cells had a lower proportion of integrin β7+ cells than ATRA+/DMSO cells. Spleens of the Vehicle group mice had a greater proportion of ATRA− than ATRA+ cells, LP of those mice had a greater proportion of ATRA+ than ATRA− cells, confirming that ATRA+ cells preferentially homed into the gut, as expected for this group. Compared to vehicle-treated mice, anti-VLA-4 treated mice had an approximately 10-fold lower proportion of ATRA+ cells in LP and approximately 2-fold lower proportion of ATRA+ cells in PP. These results confirmed that the anti-VLA-4 treatment reduced gut homing of ATRA+ cells, as expected for this positive control. The Compound A, 1000 nM group had significantly smaller proportions of CD45.1+ cells in spleens and in LP than the Vehicle group. In addition, both Compound A groups had smaller proportions of ATRA+ cells in LP compared to the Vehicle, and the reduction was close to statistically significant for the 1000 nM group.

### Methods:

Eighteen (18) B6.SJL (CD45.1+) donor mice were acclimated for 3 to 9 weeks before the start of the study and were 11 to 16 weeks old at culture setup (Day 0). On Day 0, spleen and lymph node cells were isolated from donor mice and pooled. CD3+ cells were enriched using STEMCELL Technologies kit catalog number 19851. Purity of enriched cells was confirmed by flow cytometry.

Approximately 44% of the cells were then cultured at 1.5×10⁶/mL in the presence of anti-CD3/CD28 beads (Dynabeads, ThermoFisher 11453D), with a 1:1 cell to bead ratio (ATRA−, per Table 4 below). The remaining cells were cultured under the same conditions, except that the cell concentration was 2×10⁶/mL, and either Compound A or DMSO were added the cultures. All-trans retinoic acid (ATRA) was then added to these cultures at a concentration of 0.1 μM. Table 6 below summarizes the culture conditions.

TABLE 6
Culture conditions

| | | | Test | | |
|---|---|---|---|---|---|
| | Proportion | Test | compound | | |
| Culture condition | of cells used | compound | concentration | | ATRA |
| ATRA− | 44% | — | — | | No |
| ATRA+/DMSO | 28% | Vehicle | — | | Yes |
| | | (DMSO) | | | |
| ATRA+/Compound A, | 14% | Compound A | 100 | nM | Yes |
| 100 nM | | | | | |
| ATRA+/Compound A, | 14% | Compound A | 1000 | nM | Yes |
| 1000 nM | | | | | |

On Day, 1 IL-2 was added to all cultures to reach a concentration of 30 U/mL.

From Day 2 to Day 4, cultures were expanded as needed, by adding fresh media while maintaining the following concentrations:
IL-2 at 30 U/mL for all cultures
ATRA at 0.1 μM for ATRA+ cultures
0.1% DMSO for ATRA+ cultures
Compound A as listed in Table 4

On Day 5, the cells from each culture were stained and analyzed by flow cytometry using the reagents listed in Table 7.

TABLE 7
Flow cytometry panel for evaluation
of gut homing receptor expression

| Stain | Marker |
|---|---|
| AF-700 | CD4 |
| FITC | CD44 |
| PerCP-Cy5.5 | CD62L |
| PE | PS/2 (α4, CD49d) |
| APC | FIB504 (β7) BioLegend |
| PE Cy7 | CCR9 |
| APC-Cy7 | Live cells |

Thirty-eight (38) C57BL/6 (CD45.2+) recipient mice were acclimated for 9 weeks before the start of the study (Day 0) and were 16 weeks old at cell transfer (Day 5). On Day 4, recipient mice were assigned to groups in a balanced manner to achieve similar average weight across the groups.

On Day 5, after removal of CD3/CD28 beads from cell cultures with a magnet, ATRA+ cells were labeled with CFTR, and ATRA− cells were labeled with CMFDA. Cells from each culture condition were then counted. For each group, ATRA− cells and cells from one of the ATRA+ culture conditions were mixed at a 1:1 ratio per Table 8 below and transferred into the recipient mice. Approximately 13 million of each type of cells (total of 26 million cells) were injected i.v. into each mouse.

TABLE 8
Treatment regimen

| | | | | In vivo | |
|---|---|---|---|---|---|
| Group | # mice | Group name | Transferred cells | treatment | Purpose |
| 1 | 10 | Vehicle | ATRA+/DMSO | — | Negative |
| | | | | | control |
| 2 | 8 | Anti-VLA-4 | ATRA+/DMSO | Anti- | Positive |
| | | | | VLA-4 | control |
| 3 | 10 | Compound A, | ATRA+/Compound A | — | Test |
| | | 100 nM | 100 nM | | |
| 4 | 10 | Compound A, | ATRA+/Compound A | — | Test |
| | | 1000 nM | 1000 nM | | |

Mice in Group 2 were dosed with anti-VLA-4 once, on Day 5 prior to cell transfer. Anti-VLA-4 (PS/2) antibodies were purchased from BioXCell and were kept at −80 C until needed. Antibodies were diluted to a final concentration of 1 mg/mL with sterile PBS and dosed at 10 mg/kg, intraperitoneally. No in vivo treatment was administered to other groups.

Twenty (20) to 22 hours after cell transfer all mice were euthanized, and their blood, spleen, Peyer's patches and small intestines collected. Approximately 50 μL of plasma was isolated from the blood of each mouse and sored on dry ince until further analysis.

Cells from the following tissues were isolated from each mouse for flow cytometric analysis:
Spleen
Peyer's patches
Ileal lamina propria
   Isolated cells were counted and stained with anti-CD45.1 antibodies and live/dead stain. Cells were then acquired for flow cytometric analysis and the proportions of CD45.1+, ATRA+ and ATRA− cells in each tissue determined.

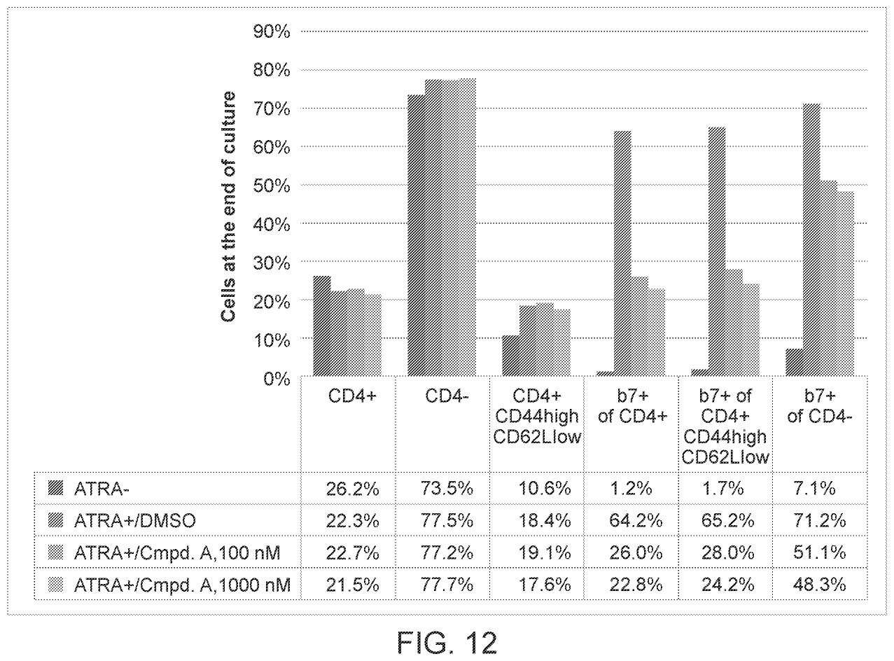

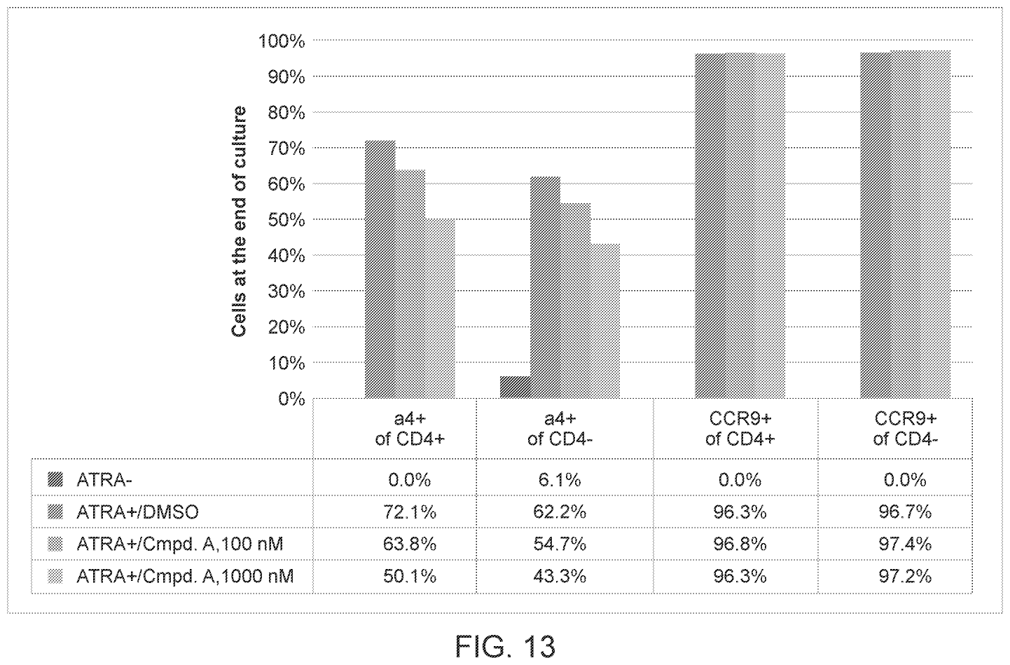

At the end of the culture period, flow cytometric analysis showed that a much smaller proportion of ATRA+/DMSO cells than ATRA− cells expressed gut homing receptors CCR9 and integrins α4 and β7 (FIGS. 12 and 13). These findings confirmed that cells cultured in the presence of ATRA upregulated gut homing receptors as expected. ATRA+/Compound A cultures had a smaller proportion of integrin β7+ cells than the ATRA+/DMSO cultures (FIG. 12). Expression of integrin α4 was also lower in the ATRA+/Compound A cultures compared to the ATRA+/DMSO cultures, but the expression of CCR9 did not appear to be affected (FIG. 13). These results indicated that Compound A either inhibited upregulation, downregulated expression or interfered with a detection of integrins β7 and α4. Results of tissue homing analysis are shown in Tables 9-13.

TABLE 9
Total number of cells isolated (×10³)

| | Total number of cells isolated (×10³) | | |
|---|---|---|---|
| Group | Spleen | Peyer's patches | Ileal LP |
| Vehicle | 119308 +/− 27168 | 1628 +/− 618 | 1560 +/− 641 |
| Anti-VLA-4 | 137208 +/− 17709 | 335 +/− 368 | 892 +/− 409 |
| p value | 0.1743 | 0.0004* | 0.0395* |
| Compound A, | 100650 +/− 10215 | 673 +/− 295 | 2005 +/− 1276 |
| 100 nM | | | |
| p value | 0.0571 | 0.0003 | 0.3372 |
| Compound A, | 90617 +/− 13418 | 775 +/− 360 | 1730 +/− 1023 |
| 1000 nM | | | |
| p value | 0.0078* | 0.0014* | 0.6612 |

*p < 0.05 vs. Vehicle

TABLE 10
CD45.1+ cells/10³ live cells

CD45.1+ cells/10³ live cells +/− SD

| Group | Spleen | Peyer's patches | Ileal LP |
|---|---|---|---|
| Vehicle | 12.7 +/− 2.2 | 0.6 +/− 0.1 | 5.9 +/− 1.9 |
| Anti-VLA-4 | 14.0 +/− 1.7 | 0.7 +/− 0.3 | 1.0 +/− 0.5 |
| p value | 0.2407 | 0.3859 | <0.0001* |
| Compound A, | 12.1 +/− 1.4 | 0.6 +/− 0.3 | 4.6 +/− 1.8 |
| 100 nM | | | |
| p value | 0.4878 | 0.5628 | 0.1463 |
| Compound A, | 10.6 +/− 1.9 | 0.6 +/− 0.1 | 3.8 +/− 1.7 |
| 1000 nM | | | |
| p value | 0.0333* | 0.4282 | 0.0192* |

*p < 0.05 vs. Vehicle

TABLE 11
ATRA+ cells and ATRA− cells/10³ live cells in spleen

| | ATRA+/10³ live | | ATRA−/10³ live | |
|---|---|---|---|---|
| Group | cells +/− SD | p value | cells +/− SD | p value |
| Vehicle | 2.9 +/− 0.7 | | 9.5 +/− 1.5 | |
| Anti-VLA-4 | 3.7 +/− 0.5 | 0.0343* | 10.1 +/− 1.2 | 0.0343* |
| Compound A, | 2.9 +/− 0.3 | 0.9734 | 9.0 +/− 1.1 | 0.3824 |
| 100 nM | | | | |
| Compound A, | 2.4 +/− 0.5 | 0.9734 | 8.0 +/− 1.4 | 0.0347* |
| 1000 nM | | | | |

*p < 0.05 vs. Vehicle

TABLE 12
ATRA+ and ATRA− cells/10³ live cells in Peyer's patches

| | ATRA+/10³ live | | ATRA−/10³ live | |
|---|---|---|---|---|
| Group | cells +/− SD | p value | cells +/− SD | p value |
| Vehicle | 0.2 +/− 0.0 | | 0.2 +/− 0.0 | |
| Anti-VLA-4 | 0.1 +/− 0.1 | 0.0003* | 0.4 +/− 0.2 | 0.0096* |
| Compound A, | 0.3 +/− 0.2 | 0.1800 | 0.2 +/− 0.2 | 0.6378 |
| 100 nM | | | | |
| Compound A, | 0.3 +/− 0.1 | 0.2762 | 0.2 +/− 0.1 | 0.4493 |
| 1000 nM | | | | |

*p < 0.05 vs. Vehicle

TABLE 13
ATRA+ and ATRA− cells/10³ live cells in Ileal LP

| | ATRA+/10³ live | | ATRA−/10³ live | |
|---|---|---|---|---|
| Group | cells +/− SD | p value | cells +/− SD | p value |
| Vehicle | 5.1 +/− 1.7 | | 0.7 +/− 0.5 | |
| Anti-VLA-4 | 0.5 +/− 0.5 | 0.0000* | 0.4 +/− 0.2 | 0.2937 |
| Compound A, | 4.3 +/− 1.7 | 0.2887 | 0.3 +/− 0.1 | 0.0278* |
| 100 nM | | | | |
| Compound A, | 3.5 +/− 1.8 | 0.0512** | 0.2 +/− 0.1 | 0.0151* |
| 1000 nM | | | | |

*p < 0.05 vs. Vehicle
**p < 0.10

The numbers of cells isolated from spleens, Peyer's patches, and ileal lamina propria (LP) in the Vehicle group were as expected (Table 9). Also, the proportions of CD45.1+ cells isolated from those tissues were as expected for this model (Table 10).

Spleens from the vehicle group had a greater proportion of ATRA− than ATRA+ cells (Table 11), while lamina propria (LP) had a greater proportion of ATRA+ than ATRA− cells (Table 13), confirming that ATRA+ cells preferentially homed into the gut, as expected for this group.

The anti-VLA-4 group had approximately 1/10^{th} the proportion of ATRA+ cells in LP and approximately ½ the proportion of ATRA+ cells in PP compared to the Vehicle mice (Tables 13 and 12), confirming that the treatment reduced gut homing of ATRA+ cells, as expected for this positive control.

The anti-VLA-4 group had significantly fewer cells isolated from Peyer's patches and ileal lamina propria than the Vehicle group (Table 9). This is typically observed in anti-VLA-4 treated mice, especially for Peyer's patches.

The proportion of ATRA+ cells in spleens of this group was significantly higher than in the Vehicle group. This is often observed in anti-VLA-4 treated mice, and may be due to ATRA+ cells being blocked from homing into gut and as a result accumulating in spleens.

Proportions of ATRA+ cells were found to be smaller in LP of mice from the Compound A groups than in the Vehicle group. This reduction was dose dependent and close to statistically significant for the cells treated with 1000 nM Compound A.

The Compound A, 1000 nM group had significantly fewer cells isolated from spleens than the Vehicle group, while both the 100 nM and the 1000 nM groups had significantly fewer cells isolated from Peyer's patches (Table 9).

The Compound A, 1000 nM group also had significantly smaller proportions of CD45.1+ cells in spleens and LP than the Vehicle group (Table 10).

Overall, these results suggest that cells treated with Compound A impaired homing into gut tissue.

### Example 6

### Compound A Inhibits Upregulation of Integrin β7

A flow cytometry-based in vitro assay was used to assess the internalization activity of peptide Compound A. The study showed that Compound A specifically causes internalization of α4β7 in human primary cells in a time- and dose-dependent manner. Compound A also causes reduction of α4β7 expression, consequently leading to decreased adhesion to MAdCAM1 by CD4⁺ T memory cells; a mean maximal 39% reduction was observed in α4β7 expression, resulting in a mean maximal 37% decrease in adhesion to MAdCAM1. Furthermore, expression of Compound A recovered to control levels after 5 days of additional incubation after removal of Compound A.

### Methods

Blood samples from human donors were obtained from the Stanford Blood Center (Stanford, Calif.) under an RB-approved research protocol. Blood was drawn into BD Vacutainer sodium heparin blood collection tubes (BD Biosciences, Cat #362753). Peripheral blood mononuclear cells (PBMCs) were isolated from blood using a SepMate-50 tube and LymphoPrep per the manufacturer's protocol. CD4⁺ T memory cells were enriched after PBMC isolation, using an EasySep kit (StemCell Technologies) per the manufacturer's protocol.

To determine specificity, human PBMCs were incubated with either 100 nM Compound C (an analog of Compound A), Compound D (an inactive triple mutant peptide analog of Compound A), or no peptide for 24 h at 37° C. in complete culture medium. After incubation, an aliquot of cells from each reaction was stained for α4β7 expression.

To determine time- and dose-dependence, purified human CD4⁺ T memory cells were incubated either with 10 nM Compound A for a range of different times (0, 1, 2, 4, 6, 24, 28, 30, and 48 h) or with different concentrations of Compound A (0, 0.01, 0.1, 1, and 10 nM) for 24 h at 37° C. in complete culture medium. After incubation, an aliquot of cells from each reaction was stained for α4β7 expression.

To determine the effects on α4β7 expression and MAdCAM1 adhesion, purified human CD4⁺ T memory cells were incubated with various concentration of Compound A (0, 0.01, 0.1, 1, and 10 nM) for 2 h at 37° C. in complete culture medium. After incubation, cells were washed extensively to remove excess peptide. For each reaction, an aliquot of cells was stained for α4β7 expression, while a separate aliquot was tested for adhesion to MAdCAM1.

To determine recovery following washout, human PBMCs were incubated with 10 nM Compound A for 24 h at 37° C. in complete culture medium (without MnCl₂). An aliquot of cells were collected before and 24 h post peptide addition and stained for α4β7 expression. Afterwards, cells were extensively washed to remove excess peptide and incubated in fresh complete culture medium (without MnCl₂) for an additional seven days. On day 1, 2, 4, 5, and 7 days after peptide washout, aliquots were stained for α4β7 expression.

Following peptide incubation, an aliquot of each reaction was stained for surface expression of α4β7 in preparation for flow cytometry. Cells were stained at 4° C. for 30 minutes, washed twice in DPBS containing 0.5% BSA (PBS/BSA), incubated with streptavidin BV421 (diluted 1:1000) at 4° C. for 30 minutes, washed twice in PBS/BSA, and then resuspended in PBS/BSA for analysis. Where relevant, a “fluorescence minus one” (FMO) sample was used as the staining control.

Samples were analyzed by flow cytometry on a BD (Franklin Lakes, N.J.) FACSVerse flow cytometer equipped with the following lasers: 405 nm (violet), 488 nm (blue), 561 nm (yellow-green), and 640 nm (red). CD4⁺ T memory cells were identified as CD4⁺, CD45RA⁺, CD197⁺ lymphocytes. α4β7 expression within CD4⁺ T memory cells was identified based on staining with the Vedolizumab BV421 complex; staining was analyzed using BD FACSuite software, version 1.0.5. Where applicable, values were normalized to no-peptide controls and expressed as percentages to permit assessment of changes in the indicated parameter. Data were plotted and analyzed using Prism software (version 7; GraphPad, La Jolla, Calif.).

### Results

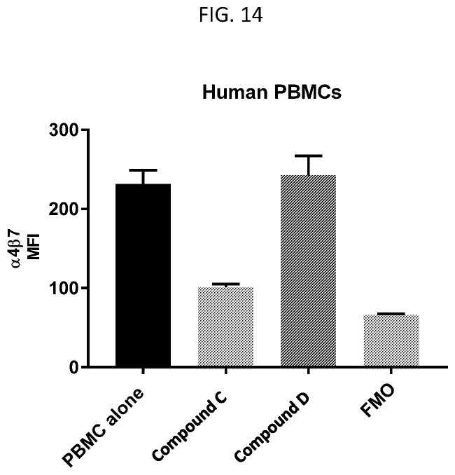

Human PBMCs were incubated with 100 nM Compound B, Compound C, or no peptide and stained for α4β7 expression. Incubation with Compound B, but not Compound C or the no-peptide control caused internalization of α4β7 (FIG. 14). These data indicate internalization is dependent on peptide binding to α4β7.

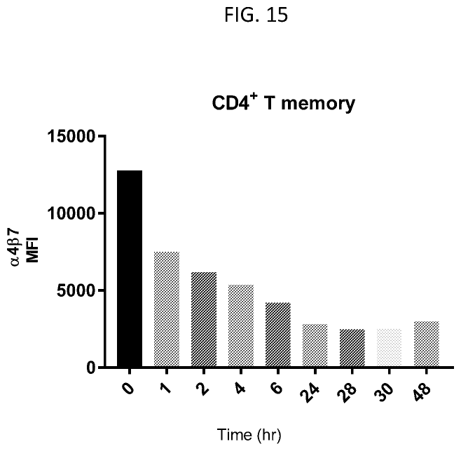

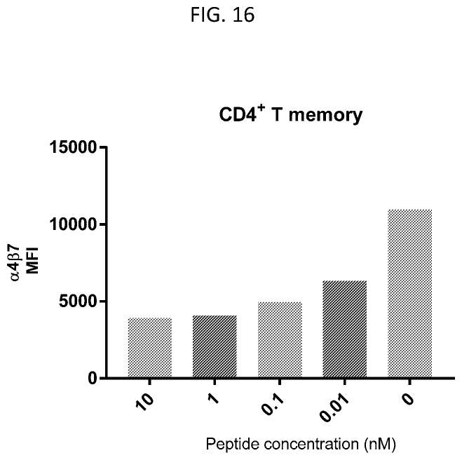

Purified human CD4⁺ T memory cells were incubated with 10 nM Compound A for a range of times (0 to 48 h) or Compound A concentrations (0-10 nM) for 24 h, and stained for α4β7 expression. The results show that Compound A induced internalization of α4β7 is time (FIG. 15) and concentration (FIG. 16) dependent, respectively. Similar results were obtained by using PBMCs (data not shown).

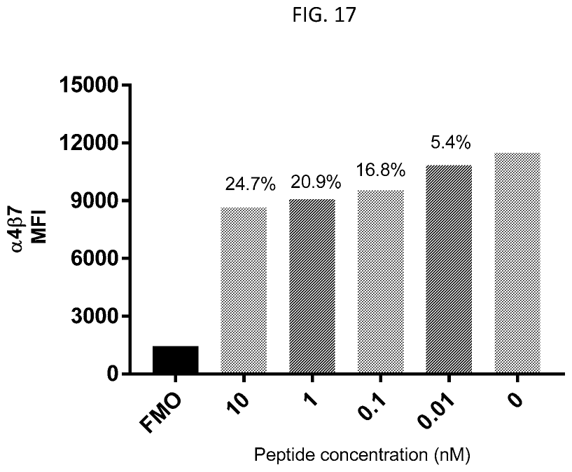

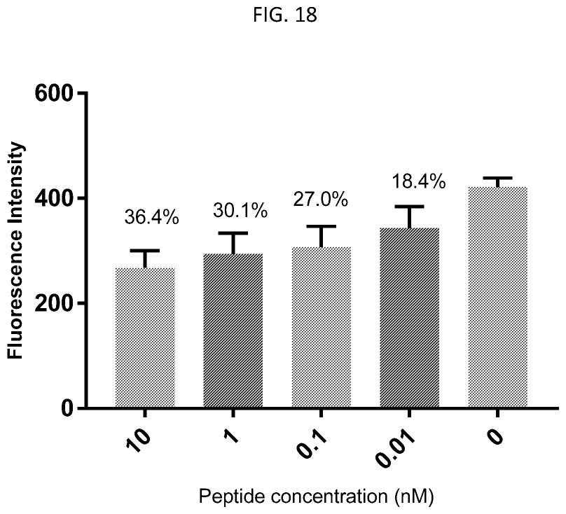

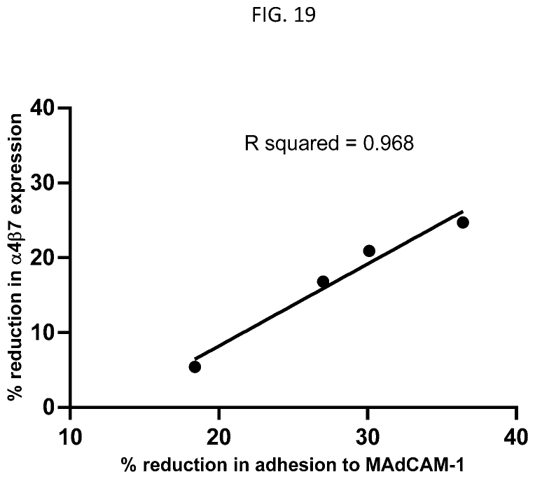

Purified human CD4⁺ T memory cells were incubated with various concentrations of Compound A, then washed to remove excess peptide. Separate aliquots from each reaction were stained for α4β7 expression and tested for MAdCAM1 adhesion, and the values normalized to the respective “no peptide treatment” controls for each assay. The data revealed that Compound A reduced expression of α4β7 (ranging from 5.4 to 24.7% decrease normalized to no peptide control) and adhesion to MAdCAM1 (ranging from 18.4% to 36.4% decrease relative to no peptide control) (FIG. 17 and FIG. 18, respectively). These effects were strongly correlated, exhibiting a R-squared value of 0.968 (FIG. 19). A similar correlation was obtained when the assays were repeated using purified human CD4⁺ T memory cells from a second donor (R squared 0.940, complete data not shown). The data from the two donors yielded a mean maximal reduction of 39% in α4β7 expression and a mean maximal decrease of 37% in adhesion to MAdCAM1, as shown in Table 1.

TABLE 14
Maximal reduction of α4β7 expression
and adhesion to MAdCAM1

| | Maximal | Maximal |
|---|---|---|
| Donor | α4β7 expression reduction (%)* | adhesion reduction (%)* |
| 1 | 53 | 38 |
| 2 | 25 | 36 |
| Mean | 39 | 37 |

*normalized to percentage of no-peptide control.

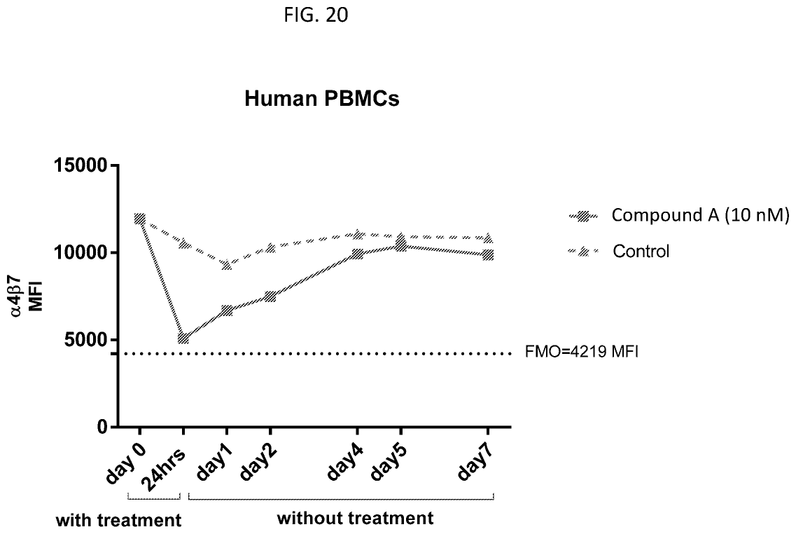

Human PBMCs were incubated with 10 nM Compound A or no peptide in the culture medium without MnCl₂ for 24 h, then washed to remove excess peptide, and resuspended in fresh medium without MnCl₂. At 24 h, α4β7 expression as measured by MFI (5090 MFI) was only 20.6% different compared to the FMO control (4219 MFI). After removal of Compound A, incubation was continued, and aliquots removed and stained for α4β7 expression at day 1, 2, 4, 5, and 7. The results showed the downregulation of α4β7 expression in the presence of peptide nearly recovered to control levels after 4-5 days of additional incubation (FIG. 20).

### Conclusions

This study showed that Compound A specifically causes internalization of α4β7 in human primary cells in a time- and dose-dependent manner. The reduction of α4β7 expression by Compound A was highly correlated with reduced adhesion to MAdCAM1 by CD4⁺ T memory cells. Internalization of α4β7 on cells by Compound A required 4-5 days of additional incubation to fully recovery to control levels of expression.

### Example 7

### Randomized, Double-Blind, Placebo-Controlled Study of Single and Multiple Ascending Doses of Compound A in Normal Healthy Volunteers

Ulcerative colitis is a chronic inflammatory bowel disease with a remitting and relapsing course, characterized by bloody diarrhea, abdominal cramps, and fatigue. The pathogenesis is thought to result from inappropriate immune response to gastrointestinal antigens and environmental triggers in genetically susceptible individuals.

The α4β7 integrin, present on the cell surface of circulating memory T- and B-lymphocytes, is primarily involved in the recruitment of leukocytes to the gastrointestinal mucosa and associated lymphoid tissues. The major ligand for α4β7, mucosal addressin cell adhesion molecule (MAdCAM1), is selectively expressed on the endothelium of the gastrointestinal vasculature and is present in increased concentrations in inflamed tissues.

Vedolizumab is an intravenously administered humanized IgG monoclonal antibody directed against α4β7 that has been approved for the treatment of moderate to severe ulcerative colitis and Crohn's disease in adult patients who are not responding to one or more conventional treatments, such as steroids, immunosuppressive agents, or tumor necrosing factor (TNF) inhibitors. Due to the inconvenience and potential systemic risks of injectable treatments, an oral, GI-restricted therapeutic that selectively targets the α4β7 integrin may provide a significant benefit to patients with ulcerative colitis. Compound A is an orally stable peptide that binds specifically to the α4β7 integrin on leukocytes and shows minimal systemic absorption (<1%) in animal studies. This study investigated the safety, tolerability, pharmacokinetics and pharmacodynamics of oral Compound A in healthy male subjects.

Two pharmacokinetic/pharmacodynamic studies were conducted in healthy volunteers. Study 1 was a first-in-human study with 40 males receiving Compound A, 100- to 1400 mg or placebo, as single doses and 57 males receiving Compound A, 100- to 1000 mg or placebo, as multiple doses. Study 2 was a randomized, crossover study comparing multiple doses of 450 mg Compound A twice daily as a liquid solution and as an immediate-release tablet in 10 subjects.

No subjects discontinued due to treatment-emergent adverse events. Consistent with the gastrointestinal-restricted nature of the peptide, systemic exposure was minimal; there was an approximate dose proportional increase in AUC. There was minimal accumulation with once-daily dosing and an absence of time-dependent changes in pharmacokinetics. Administration of Compound A after a high fat meal reduced peak plasma concentration and AUC. There was minimal (<0.1%) urinary excretion of intact drug and there was a dose-related increase in fecal excretion of intact Compound A. Dose-dependent increases in blood receptor occupancy and reduction in blood receptor expression were observed, supporting target engagement. Twice daily dosing resulted in sustained receptor occupancy with low plasma fluctuations (143%).

Compound A was generally well tolerated following single and multiple oral doses with low systemic exposure. Twice daily dosing resulted in sustained pharmacokinetics and pharmacodynamics, supporting further investigation in efficacy studies.

### Methods

### Study Design

Two studies were conducted at a single clinical center.

Study 1 was a three-part first in human study in healthy male volunteers to assess the safety, tolerability, pharmacokinetics, and pharmacodynamic of a liquid solution formulation of COMPOUND A.

Part 1 was a randomized, placebo-controlled, double-blind study of single ascending doses of COMPOUND A in 40 males divided into 4 equal cohorts. Dose escalation proceeded from 100 mg, 300 mg, 1000 mg, 1400 mg. Subjects in the 300 mg dose cohort received treatment in the fasted state on one occasion and following a high fat meal on a second occasion in a crossover fashion. The high fat meal consisted of two eggs fried in butter, two strips of bacon, two slices of toast with butter, four ounces of hash brown potatoes and 240 ml of whole milk. During Part 1, subjects refrained from food and drink except water for 10 hours before and for four hours after dosing with the exception of subjects in the 300 mg dose cohort during the fed treatment.

Part 2 was a randomized, placebo-controlled, double-blind multiple ascending dose study in 50 male subjects divided equally into 5 cohorts. Subjects received once-daily dosing of COMPOUND A or placebo for 14 days. Doses evaluated in Part 2 included 100 mg, 300 mg and 1000 mg. During Part 2, two cohorts of subjects (100 mg and 300 mg) received food approximately 30 minutes prior to each dose and another two cohorts of subjects (300 mg and 100 mg) refrained from food for 10 hours before and for 1 hour after dosing. An additional cohort of 9 subjects in Part 2 received 300 mg COMPOUND A in a crossover fashion to evaluate the effect of meal timing on the pharmacokinetics and pharmacodynamics of COMPOUND A. Subjects in this cohort received a meal 30, 60 or 90 minutes following COMPOUND A dosing.

Part 3 was an open-label, randomized, crossover multiple-dose comparison of 900 mg once-daily and 450 mg twice-daily dosing of COMPOUND A as a liquid solution for five days. Subjects in Part 3 refrained from food for 10 hours before and for 1 hour after dosing of COMPOUND A.

The second study was a 5-day multiple-dose pharmacokinetic and pharmacodynamic study comparing the liquid formulation and a tablet formulation administered as 450 mg COMPOUND A twice daily in healthy males and females. Subjects held food for 10 hours before and for 1 hour after the morning dose and for one hour before and after the evening dose of each day.

The study protocols, subject information and informed consent form were reviewed and approved by independent human research ethics committees. The studies were conducted in accordance with the Declaration of Helsinki on biomedical research involving human subjects and International Conference on Harmonization Good Clinical Practice guidelines and all study procedures were conducted by scientifically and medically qualified personnel. Written informed consent explaining the nature, purpose and potential risks and benefits of the study was provided by subjects prior to any study-related activities.

### Study Subjects

Both studies used similar procedures for screening and enrollment. Subjects were screening within 21 days of enrollment. Eligible subjects were aged 18 to 55 years inclusive with a body mass index (BMI) between 18-30 kg/m², who were in good general health, with no significant medical history or clinically significant abnormalities on physical examination. The first-in-human study (Study 1) enrolled only males while the study evaluating the tablet formulation (Study 2) enrolled men and women who agreed to use highly effective methods of contraception based on the Clinical Trials Facilitation and Coordination Group for the duration of the study and for 90 days after the last dose.

Subjects were excluded if they had a history of clinically significant endocrine, gastrointestinal cardiovascular, hematologic, hepatic, immunologic, renal respiratory, or genitourinary abnormalities or diseases, or had clinically significant laboratory abnormalities, including impaired renal function (serum creatinine >106 umol/L or estimated creatinine clearance <80 mL/min) or alanine aminotransferase or aspartate aminotransferase values >1.2 times the upper normal limits.

### Procedures

Study 1: The single and multiple ascending dose phase of the study consisted of sequential dose escalations in 10 subject per dose cohort. Participants were randomized to receive COMPOUND A or matching placebo as a 60 mL oral solution in a ratio of 8:2. Dose solutions were formulated in 50 mM phosphate buffer pH 7.4 and were prepared weekly by a qualified pharmacist. Dosing solutions over the anticipated concentration range were demonstrated to be stable for 3 months when stored at 2-8° C.

Blood samples for pharmacokinetics were collected predose and for 48 hours postdose following single doses. In the multiple ascending dose phase, blood samples were obtained on Days 1-3 and 14-16; on Days 8 samples were obtained predose, 4, and 12 hours. On Day 10 of the MAD, subjects were required to collect all urine for the 0-6, 6-12, 12-18, and 18-24 hour intervals postdose and on Day 11 subjects were required to collect fecal samples.

The decision to proceed to the next dose level was made by the investigator and the safety monitoring committee based on acceptable safety and tolerability of the lower dose.

Study 2: This study was a randomized, open-label, two treatment, two period, multiple dose study to determine the safety, tolerability, pharmacokinetics and pharmacodynamics of an immediate-release (IR) tablet and a liquid solution of COMPOUND A. The study allowed comparison of a solid dose formulation to the liquid formulation that had investigated in the first-in-human study. Subjects received 450 mg COMPOUND A twice daily (BID) for 5 days as one 300 mg and one 150 mg dosage strength IR tablet administered every 12 hours and 450 mg COMPOUND A BID for 5 days as a liquid solution administered every 12 hours in a randomized fashion.

### Dosing

The starting dose in the first in human single and multiple dose study was based on consideration of the no observed effect level (NOEL) from 28-day toxicology studies in rats and cynomolgus monkeys, and the receptor occupancy noted in cynomolgus monkeys. The NOEL determined in rats and monkeys translated to a human equivalent dose of approximately 145 mg using standard allometric scaling and a 10-fold safety margin. A starting dose of 100 mg was selected with initial stepwise escalations of approximately 3-fold.

The dose selected for Study 2, comparing a tablet and the oral solution formulation, was based on the pharmacokinetic and pharmacodynamic profile from Part 3 of Study 1 and the anticipated dose planned in an efficacy study in patients with moderate to severe ulcerative colitis.

### Analytical Methods

Concentrations of COMPOUND A in plasma, urine and fecal samples from Study 1 and plasma and urine samples from Study 2 were assayed using a validated high-performance liquid chromatography tandem mass spectrometry (LC/MS/MS) method. The drug and internal standard were extracted from the matrix by a protein precipitation procedure. The limit of quantitation was 0.2 ng/mL, 20 ng/mL, and 100 ng/mL in plasma, urine, and feces, respectively. Sample stability was demonstrated for at least 100 days and for 4 freeze-thaw cycles for all matrices. Coefficient of determinations for the calibration curves were at least 0.99 for all matrices. The interassay accuracy (% bias) ranged from −2.2% to 1.0% for plasma, −3.8% to 9.0% for urine and −5.0 to 5.2% for feces. Interassay precision (% CV) ranged from 3.7% to 7.7% for plasma, 2.8% to 7.0% for urine, and 1.2% to 5.2% for feces. Reanalysis of incurred samples indicated >88% of samples with valid reanalyses met acceptable criteria indicating that the analytical methods were acceptable.

### Study Endpoints

The primary endpoint is the first-in-human study was the safety and tolerability assessments following single and multiple dosing with COMPOUND A. Secondary objectives were to characterize the pharmacokinetics and pharmacodynamics, evaluate the effect of a high-fat meal on COMPOUND A pharmacokinetics, and compare twice-daily and once-daily dosing. Safety assessments, adverse events, and laboratory assessments are summarized descriptively for the placebo and each COMPOUND A dose.

The endpoints for the second study comparing the oral solution and the tablet formulation were pharmacokinetics and pharmacodynamics.

### Pharmacokinetic Analyses

Pharmacokinetic parameters were estimated by noncompartmental methods using Phoenix WinNonlin (Certara, Princeton N.J.). Peak plasma concentration (Cₘₐₓ) and time to peak plasma concentration (Tₘₐₓ) were observed values. The elimination rate was estimated from the slope of the least-squares regression on the terminal log-linear phase. Area under the plasma concentration-time curve from time zero to the last quantifiable concentration (AUCₜ) was estimated by a linear trapezoidal method and was extrapolated to infinity (AUC_{∞}) by dividing the last quantifiable concentration b the elimination rate. Fluctuation in the steady-state plasma concentration was calculated as

$$\frac{{C}_{max} - {C}_{min}}{{C}_{average}} .$$

### Pharmacodynamic Assays for α4β7 Receptor Occupancy and Receptor Expression

Translational biomarkers such as receptor occupancy have been validated as pharmacodynamic markers through use in preclinical studies and in clinical trials with vedolizumab.²⁷⁻²⁸ In this study, a flow cytometry-based assay was designed to quantify the amount of α4β7 integrin on the cell surface that is occupied by COMPOUND A or the amount of α4β7 expression on the cell surface of circulating lymphocytes in response to engagement by COMPOUND A. Briefly, in this assay, each heparinized whole blood sample is first treated with saturating amount of an unlabeled competing peptide serving as the “blocked” control for 100% receptor occupancy, or no peptide serving as the “unblocked” sample to measure the level of blocking by orally administered COMPOUND A. After incubation, the blood is stained with a sub-saturating concentration of Alexa647-labelled peptide, followed by staining with the cell surface marker panel (CD45, CD3, CD4, CD45RA, CD19, IgD and the anti-α4β7 antibody vedolizumab). After staining is completed, the samples are treated with a red blood cell lysis and fixation buffer, washed and acquired on a flow cytometer. To quantify receptor occupancy on α4β7 expressing memory CD4 T cells, the medium fluorescence intensity (MFI) of Alexa647-labelled peptide within the vedolizumab+ memory CD4+ T cells was used. Receptor occupancy was calculated according to the following formula: [Percent RO]=(1−([Unblocked]−[Blocked])/([Baseline Unblocked]−[Baseline Blocked]))×100.

Expression of α4β7 is defined by MFI of vedolizumab within the memory CD4+ T cells from the unblocked samples. Receptor expression (RE) was calculated as percent change of MFI from baseline for the vedolizumab stain.

### Statistical Analyses

No formal sample size estimations were performed. Eight subjects received oral COMPOUND A and 2 subjects received placebo in each dose cohort in the single- and multiple-ascending dose study. Ten subjects were enrolled in the second study comparing the immediate-release tablet formulation to the oral solution. The enrollment in each study was considered adequate to assess the tolerability and safety and to allow characterization of the pharmacokinetics and pharmacodynamics of COMPOUND A.

### Results

### Subject Characteristics and Disposition

A total of 97 healthy male subjects were enrolled in Study 1 with 40 subjects enrolled in the single dose phase and 57 subjects in the multiple dose phase. 95 subjects completed their dosing with COMPOUND A or placebo as planned. Two subjects withdrew consent for personal reasons unrelated to safety; one subject did not want to remain in the clinical unit and a second subject was uncomfortable with the venous cannula. The average age was 28.7 years in the single dose phase and 30.9 years in the multiple dose phase.

Ten subjects were enrolled in Study 2 and nine subjects completed both treatments. One subject discontinued the study on Day 1 following the oral solution treatment due to an adverse event of acute tonsillitis that was considered unrelated to study drug.

### Safety and Tolerability

A total of 23 TEAEs were reported by 14 subjects during the single ascending dose phase. Of the 13 subjects who experienced TEAEs, 12 received COMPOUND A (21 events) and 2 received placebo (2 events). All TEAEs were mild or moderate except for a severe headache in a subject treated with 100 mg COMPOUND A that was not considered related to treatment. All subjects recovered from the AEs and no subjects were withdrawn due to AEs. No clinically relevant changes were observed in respiratory rate or vital signs, clinical laboratory parameters (hematology, coagulation, serum chemistry, or urinalysis), or in the interpretation of electrocardiograms or QTc interval.

### Safety and Tolerability

Thirty subjects in the group receiving multiple doses of COMPOUND A reported a total of 68 AEs. All but two occurrences were mild in severity. One report of upper respiratory tract infection was characterized as moderate and one report of influenza that occurred after release from the clinical unit was categorized as severe and considered a serious adverse event. Four subjects receiving placebo reported a total of 6 mild TEAEs, primarily gastrointestinal disorders. Treatment-emergent adverse events reported in 2 or more subjects in the multiple ascending dose phase included abdominal discomfort, flatulence, upper respiratory tract infection, back pain, dizziness, and headache. Nervous system disorders, particularly headache, were the most commonly reported TEAEs. No clinically relevant changes were observed in respiratory rate, vital signs, clinical laboratory parameters, or on the electrocardiograms.

### Safety and Tolerability

Of the 10 subjects enrolled in Study 2 comparing dosing with immediate-release tablets to an oral solution, nine subjects completed both treatments. One subject experienced an adverse event of moderate tonsillitis unrelated to the treatment that let to discontinuation from the study. The incidence of treatment-emergent adverse events was similar across both treatments. The most common adverse event was headache with all other adverse events being reported in only one subject.

### Safety and Tolerability

### Pharmacokinetics

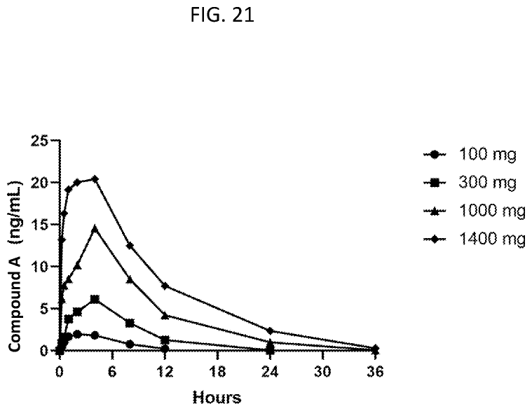

The mean plasma concentration-time profiles following single doses of COMPOUND A are presented in FIG. 21. The single dose pharmacokinetics of COMPOUND A are summarized in Table 15.

TABLE 15
Single-Dose Pharmacokinetics of Compound A (Mean ± SD)

| | | 300 mg | 300 mg | | |
|---|---|---|---|---|---|
| | 100 mg | Fasted | High Fat Meal | 1000 mg | 1400 mg |
| | (N = 8) | (N = 8) | (N = 8) | (N = 8) | (N = 8) |
| Cmax (ng/mL) | 2.11 ± 1.15 | 6.55 ± 3.38 | 1.58 ± 0.71 | 15.3 ± 4.11 | 23.5 ± 19.0 |
| Tmax (h)^{a} | 2.0 (1.0, 4.0) | 3.0 (1.0, 8.0) | 4.0 (2.0, 4.0) | 4.0 (0.5, 4.0) | 4.0 (1.0, 12.0) |
| AUCt (ng · h/mL) | 12.9 ± 7.27 | 44.3 ± 21.5 | 11.5 ± 4.62 | 138 ± 33.7 | 257 ± 173 |
| AUCinf (ng · h/mL) | 16.5 ± 8.70^{b} | 57.6 ± 23.6^{b} | —^{c} | 151 ± 31.7^{d} | 260 ± 176 |
| t½ (h) | 3.05 ± 0.71^{b} | 4.02 ± 1.37^{b} | —^{c} | 5.26 ± 0.91^{d} | 5.74 ± 1.35 |

^{a}Median (Min, Max)
^{b}N = 4
^{c}Not reported due to insufficient data
^{d}N = 7

Median time to peak plasma concentration was 2 to 4 hours. Mean peak COMPOUND A plasma concentration (Cₘₐₓ) increased from 2.11 mg/mL to 23.5 ng/mL and AUC_{inf} increased from 16.5 ng·h/mL to 260 ng·h/mL as COMPOUND A doses increasing from 100 mg to 1400 mg. There was a dose-proportional increase in AUC_{inf} and a slightly less than dose-proportional increase in Cₘₐₓ over the dose range of 100 mg to 1400 mg COMPOUND A. The mean elimination half-life at the lower doses (100 and 300 mg) was 3.1 to 4.0 hours and at higher doses (1000 and 1400 mg) was 5.3 to 5.7 hours.

### Safety and Tolerability

The pharmacokinetics of COMPOUND A following multiple doses is summarized in Table 16.

TABLE 16
Multiple-Dose Pharmacokinetics of Compound A (Mean ± SD)

| | 100 mg Fed | | 300 mg Fed | | 300 mg Fasted | | 1000 mg Fasted | |
|---|---|---|---|---|---|---|---|---|
| | (N = 8) | | (N = 8) | | (N = 8) | | (N = 8) | |
| | Day 1 | Day 14 | Day 1 | Day 14 | Day 1 | Day 14 | Day 1 | Day 14 |
| Cmax | 0.745 ± 0.409 | 0.898 ± 0.454 | 2.32 ± 1.34 | 2.80 ± 1.66 | 7.23 ± 4.29 | 4.72 ± 1.85 | 13.8 ± 3.86 | 17.1 ± 6.05 |
| (ng/mL) | | | | | | | | |
| Tmax (h)^{a} | 4.0 (2.0, 8.0) | 4.0 (2.0, 4.0) | 4.0 (1.0, 4.0) | 4.0 (4.0.8.0) | 2.0 (0.5, 2.0) | 2.0 (1.0, 4.0) | 2.0 (0.25, 4.0) | 2.0 (1.0, 4.0) |
| AUCt | 4.58 ± 3.12 | 5.87 ± 3.38 | 16.4 ± 10.8 | 21.0 ± 9.99 | 42.9 ± 17.9 | 39.5 ± 11.5 | 141 ± 43.6 | 184 ± 81.8 |
| (ng · h/mL) | | | | | | | | |
| AUCinf | 9.81^{b} | 8.33^{b} | 39.7^{b} | —^{c} | 46.3 ± 19.2 | 43.9 ± 13.0^{d} | 157 ± 55.7 | 207 ± 125 |
| (ng · h/mL) | | | | | | | | |
| t½ (h) | 3.85^{b} | 7.40^{b} | 7.96^{b} | —^{c} | 5.16 ± 1.92 | 7.31 ± 3.86^{d} | 6.60 ± 1.99 | 7.70 ± 3.69 |

^{a}Median (Min, Max)
^{b}N = 1
^{c}Not reported due to insufficient data
^{d}N = 7

There was an approximate dose-proportional increase in Cₘₐₓ and in AUC_{inf} for the 100 and 300 mg dose groups in the fed condition on Day 14 and between the 300 mg and 1000 mg dose groups in the fasted condition on Day 14. Median time to peak plasma concentration ranged from 2 to 4 hours. The mean elimination half-life was 5.2 to 7.7 hours. Consistent with the half-life, a comparison of the Cₘₐₓ and the AUCₜ values for individual subjects on Day 1 and on Day 14 at 300 mg and 1000 mg suggested minimal (≤30%) accumulation with once daily dosing. Comparison of the AUCinf values on Day 1 and AUCₜ values on Day 14 indicated the absence of time-dependent changes in the pharmacokinetics of COMPOUND A.

During the multiple ascending dose phase, 24-hour collection of urine and feces as undertaken in the 300 mg and 1000 mg dose groups. Only a small fraction of COMPOUND A was recovered intact in urine over 24 hours, with recoveries of 0.028%, 0.056%, and 0.056% in the 300 mg fasted, 300 mg fed, and 1000 mg dose groups, respectively. There was a dose-related increase in the 24-hour fecal recovery of Compound A with 0.73%, 1.78%, and 16.8% COMPOUND A recovered intact in the 300 mg fasted, 300 mg fed, and 1000 mg dose groups, respectively.

### Effect of Food

The effect of a high-fat meal on the pharmacokinetics of COMPOUND A was evaluated in a crossover fashion at 300 mg during the single ascending dose portion of Study 1.

Administration of COMPOUND A within 30 minutes of consuming a high-fat meal reduced the peak concentration and exposure compared to the fasted state (Table 13). Mean COMPOUND A peak plasma concentrations were 6.55 ng/mL in the fasted state and 1.58 ng/mL in the fed state. Median time to peak concentration was delayed by one hour following a high fat meal.

The effect of the interval between COMPOUND A dosing and consumption of a meal was examined in Study 1. Subjects received a meal 30, 60, or 90 minutes following a single dose of 300 mg COMPOUND A. The median time to peak COMPOUND A plasma concentrations was 1, 2, and 4 hours for the 30, 60, and 90-minute treatment groups. There was a small increase in the Cₘₐₓ and AUCₜ values when food was delayed 60 or 90 minutes compared to 30 minutes following COMPOUND A with minor differences noted between the 60- and 90-minute delay. Based on the more favorable Cₘₐₓ and AUCₜ values noted for the 60 minute delay in food compared to the 30 minute delay, dosing for additional cohorts in the multiple ascending dose incorporated a one hour fasting interval before and after dosing of COMPOUND A.

Table 16 presents a comparison of the pharmacokinetics of 300 mg COMPOUND A following an overnight fast compared to refraining from a meal within 1 hour of dosing COMPOUND A as part of the multiple ascending dose phase of Study 1. The median time to peak concentration was 4 hours following an overnight fast whereas it was 2 hours when food was consumed 1 hour following COMPOUND A. Peak plasma concentrations were lower when COMPOUND A was administered following an overnight fast compared to when food was administered 1 hour following dosing (Day 1 Cₘₐₓ were 7.23 ng/mL and 2.32 ng/mL for the fasted and fed 1 hour post dose case, respectively).

### Pharmacokinetics of Once-Daily and Twice-Daily Dosing

The effect of dosing regimen was evaluated following 900 mg once daily for 5 days and 450 mg twice daily for 5 days in a randomized crossover fashion in Part 3 of Study 1.

FIG. 25 presents the mean plasma concentration-time profiles following 900 mg once daily and 450 mg twice daily dosing of COMPOUND A. A summary of the pharmacokinetic comparing once-daily and twice daily dosing is presented in Table 17.

TABLE 17
Pharmacokinetics of Compound A following Once-
Daily and Twice-Daily Dosing (Mean ± SD)

| | 900 mg Once Daily (N = 7) | | 450 mg Twice Daily (N = 8) | |
|---|---|---|---|---|
| | Day 1 | Day 5 | Day 1 | Day 5 |
| Cmax (ng/mL) | 22.8 ± 16.9 | 14.2 ± 4.84 | 5.98 ± 1.61 | 9.96 ± 5.82 |
| Tmax (h)^{a} | 2.0 (1.0, 8.0) | 2.0 (1.0, 4.0) | 2.0 (2.0, 4.0) | 2.0 (1.0, 4.0) |
| AUCt (ng · h/mL) | 159 ± 70.5 | 140 ± 74.4 | 77.6 ± 37.1 | 132 ± 106 |
| AUCinf (ng · h/mL) | 176 ± 98.5 | 160 ± 96.5 | —^{b} | — |
| AUCtau (ng · h/mL) | 159 ± 70.5 | 140 ± 74.4 | 41.5 ± 10.7 | 72.1 ± 54.7 |
| t½ (h) | 5.27 ± 2.40 | 6.24 ± 2.97 | — | — |
| C_{trough} (ng/mL) | — | 1.78 ± 2.3 | — | 3.25 ± 3.5 |
| Accumulation Cmax | — | 0.87 ± 0.5 | — | 1.56 ± 0.6 |
| Accumulation AUCt | — | 0.88 ± 0.3 | — | 1.66 ± 0.9 |
| Fluctuation (%) | — | 245 ± 81 | — | 143 ± 42 |

^{a}Median (min, max)
^{b}Not reported due to insufficient data

Peak concentrations were noted at a median of 2 h for both dosing regimens on Day 1 and on Day 5. The steady-state peak concentrations were 14.2 ng/mL for once daily dosing and 9.96 ng/mL for twice daily dosing. Dose-adjusted area under the curve over the dosing interval was comparable for the two treatment regimens. Consistent with the half-life of COMPOUND A, there was minimal accumulation with once daily dosing and the accumulation was approximately 1.6- to 1.7-fold with twice-daily dosing. Twice daily dosing of 450 mg COMPOUND A as a liquid solution resulted in sustained plasma concentrations as reflected by the lower peak-to-trough fluctuation (143% versus 245%) and higher trough concentrations (3.25 ng/mL versus 1.78 ng/mL) compared to 900 mg once daily (Table 18).

### Pharmacokinetics of a Liquid Solution and an Immediate-Release Tablet Formulation of COMPOUND A

The steady-state pharmacokinetics of an immediate-release tablet of COMPOUND A administered as 450 mg twice daily for 5 days compared to the liquid solution used in the first-in-human study is summarized in Table 18.

TABLE 18
Steady-State Pharmacokinetics of Compound A following
Oral Dosing of 450 mg Twice-Daily as a Liquid
Solution and as an IR Tablet (Mean ± SD)

| | Liquid Solution | IR Tablet |
|---|---|---|
| | 450 mg BID (N = 9) | 450 mg BID (N = 9) |
| Cₘₐₓ (ng/mL) | 9.36 ± 4.81 | 7.67 ± 2.97 |
| Tₘₐₓ (h)^{a} | 2.0 (1.0, 8.0) | 2.0 (2.0, 4.0) |
| AUC₀₋₂₄ (ng · h/mL) | 106 ± 34.7 | 86.3 ± 30.9 |
| AUC₀₋₁₂ (ng · h/mL) | 65.6 ± 38.6 | 53.8 ± 20.9 |
| AUC₁₂₋₂₄ (ng · h/mL) | 39.3 ± 15.2 | 32.4 ± 11.4 |
| Bioavailability (%) | — | 85.3 ± 36.2 |
| Accumulation Cₘₐₓ | 1.25 ± 0.46 | 2.19 ± 1.0 |
| Accumulation AUC | 1.36 ± 0.33 | 1.57 ± 0.43 |
| C_{trough} (ng/mL) | 1.98 ± 0.83 | 1.86 ± 0.97 |

^{a}Median (Min, Max)

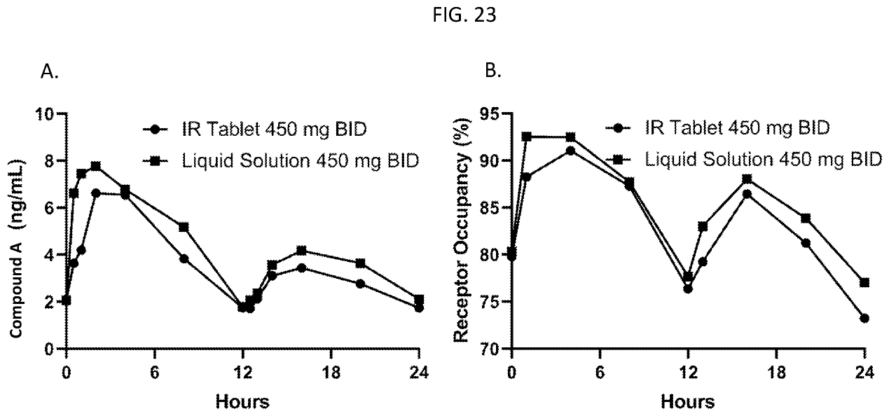

FIG. 23A presents the mean steady-state plasma concentration-time profile for the two formulations. Both formulations had a similar median time to peak concentration (2 hours) while the peak concentration was approximately 20% lower for the IR tablet compared to the liquid solution. The IR tablet formulation had bioavailability of approximately 85% relative to the liquid solution. Twice daily dosing of the tablet formulation resulted in an accumulation of approximately 2-fold based on Cₘₐₓ and 1.6-fold based on AUC. Steady-state trough concentrations of COMPOUND A were comparable for the IR tablet and the liquid solution (1.86 ng/mL and 1.98 ng/mL, respectively).

### Pharmacodynamics

The mean pharmacodynamics of α₄β₇⁺ memory CD4⁺ T cell as measured by mean percent receptor occupancy and mean receptor expression following single doses of COMPOUND A is summarized in Table 19.

TABLE 19
Pharmacodynamics following Single Dose of Compound A (Mean ± SD).

| | | | | |
|---|---|---|---|---|
| Placebo | 100 mg | 300 mg | 1000 mg | 1400 mg |
| (N = 8) | (N = 8) | (N = 8) | (N = 8) | (N = 8) |

Receptor Occupancy

| | | | | | |
|---|---|---|---|---|---|
| ROₘₐₓ (%) | 8.6 ± 7.0 | 61.8 ± 10.8 | 83.4 ± 7.92 | 93.6 ± 2.04 | 94.8 ± 3.55 |
| Tₘₐₓ for RO (h) | 12 ± 11 | 3.8 ± 2.2 | 4.6 ± 2.3 | 4.1 ± 1.9 | 4.6 ± 3.2 |
| Average RO (%) | 1.7 ± 4.9 | 38.9 ± 12.5 | 64.3 ± 6.6 | 81.0 ± 3.5 | 86.0 ± 6.8 |

Receptor Expression

| | | | | | |
|---|---|---|---|---|---|
| REₘₐₓ (%) | −5.06 ± 11.7 | −28.2 ± 7.97 | −43.6 ± 3.69 | −45.4 ± 3.70 | −49.0 ± 8.20 |
| Tₘₐₓ for RE (h) | 18 ± 9.1 | 11 ± 6.0 | 12 ± 0 | 12 ± 0 | 12 ± 0 |
| Average RE (%) | −1.7 ± 4.9 | −21.6 ± 7.1 | −32.4 ± 3.0 | −36.3 ± 2.9 | −40.3 ± 6.9 |

ROₘₐₓ maximum receptor occupancy, REₘₐₓ maximum receptor expression

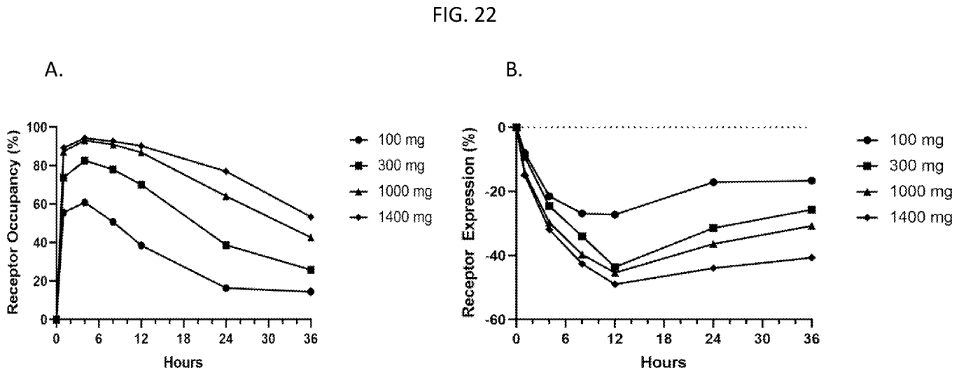

The time course of the mean percent receptor occupancy and mean receptor expression following single doses is presented in FIGS. 22A-B. The mean time to peak α₄β₇ memory CD4⁺ T cell receptor occupancy was approximately 4 hours. Mean peak receptor occupancy increased in a dose-related manner, ranging from 61.8% at 100 mg to 94.8% at 1400 mg. Peak receptor occupancy for the 1000 mg and 1400 mg dose cohorts was similar, indicating that receptor occupancy saturation was achieved by single doses of approximately 1000 mg COMPOUND A. Mean change in receptor expression (REₘₐₓ) increased with dose, ranging from −28.2% for 100 mg COMPOUND A to −49.0% for the 1400 mg dose group (Table 19).

α₄β₇⁺ memory CD4⁺ T cell percent receptor occupancy following multiple doses of COMPOUND A is presented in Table 20.

TABLE 20
Multiple Dose Pharmacodynamics of Compound A (Mean ± SD)

| Placebo | | 100 mg Fed | | 300 mg Fed | |
|---|---|---|---|---|---|
| (N = 8) | | (N = 8) | | (N = 8) | |
| Day 1 | Day 14 | Day 1 | Day 14 | Day 1 | Day 14 |

Receptor Occupancy

| | | | | | | |
|---|---|---|---|---|---|---|
| ROₘₐₓ (%) | 10.6 ± 15.0 | 16.3 ± 21.2 | 46.8 ± 11.2 | 55.5 ± 8.82 | 70.4 ± 7.39 | 78.9 ± 5.89 |
| Tₘₐₓ for | 9.5 ± 4.8 | 21 ± 17 | 6.0 ± 3.7 | 5.0 ± 2.8 | 4.0 ± 0 | 4.0 ± 0 |
| RO (h) | | | | | | |
| Average | 5.0 ± 13.0 | 8.0 ± 15.8 | 30.0 ± 7.7 | 40.5 ± 7.5 | 47.2 ± 7.5 | 57.8 ± 3.9 |
| RO (%) | | | | | | |

Receptor Expression

| | | | | | | |
|---|---|---|---|---|---|---|
| REₘₐₓ (%) | −0.85 ± 12.7 | −9.8 ± 19.8 | −17.4 ± 7.5 | −29.1 ± 7.2 | −28.4 ± 4.4 | −37.2 ± 4.7 |
| Tₘₐₓ for | 12.0 ± 8.3 | 9.5 ± 8.0 | 14.0 ± 4.2 | 12.0 ± 0 | 12.0 ± 0 | 12.0 ± 0 |
| RE (h) | | | | | | |
| Average | 0.06 ± 7.8 | −5.9 ± 11.0 | −12.3 ± 6.6 | −23.8 ± 6.9 | −20.1 ± 3.4 | −31.7 ± 4.0 |
| RE (%) | | | | | | |

| | 300 mg Fasted | | 1000 mg Fasted | |
|---|---|---|---|---|
| | (N = 8) | | (N = 8) | |
| | Day 1 | Day 14 | Day 1 | Day 14 |
| Receptor Occupancy | | | | |
| ROₘₐₓ (%) | 77.8 ± 5.13 | 79.7 ± 5.40 | 91.3 ± 2.93 | 95.6 ± 0.97 |
| Tₘₐₓ for | 4.0 ± 0 | 4.0 ± 0 | 7.0 ± 4.1 | 4.0 ± 0 |
| RO (h) | | | | |
| Average | 53.9 ± 6.5 | 61.1 ± 6.0 | 77.1 ± 3.7 | 81.5 ± 15.5 |
| RO (%) | | | | |
| Receptor Expression | | | | |
| REₘₐₓ (%) | −33.1 ± 5.2 | −41.5 ± 5.9 | −43.1 ± 4.0 | −57.3 ± 2.9 |
| Tₘₐₓ for | 12.0 ± 0 | 6.0 ± 3.7 | 12.0 ± 0 | 4.0 ± 0 |
| RE (h) | | | | |
| Average | −25.1 ± 4.4 | −37.7 ± 5.2 | −33.9 ± 3.4 | −51.3 ± 3.1 |
| RE (%) | | | | |

ROₘₐₓ maximum receptor occupancy,
REₘₐₓ maximum receptor expression

Mean peak memory T cell receptor occupancy following multiple doses of COMPOUND A achieved a peak at approximately 4 hours. Mean percent receptor occupancy on Day 1 of the multiple dose cohorts were comparable to the corresponding doses in the single dose cohorts. On Day 1, the mean peak receptor occupancy following 300 mg and 1000 mg was 77.8% and 91.3%, respectively. There was a small increase in the peak percent receptor occupancy with continued daily dosing over 14 days. On Day 14, the mean peak receptor occupancy in following 300 mg and 1000 mg was 79.7% and 95.6%, respectively.

Administration of COMPOUND A within 30 minutes of consuming a high fat meal reduced the pharmacodynamic effect, consistent with the effect of a high fat meal on the pharmacokinetics. Peak percent receptor occupancy following 300 mg COMPOUND A in the fasted state was 83.4% compared to 61.4% when COMPOUND A was administered within 30 minutes following a high fat meal. Delaying food for 60 minutes following COMPOUND A administration improved the pharmacodynamic profile compared to consuming a meal within 30 minutes following dosing or dosing COMPOUND A following a high fat meal. There was relatively little difference in the steady-state (Day 14) pharmacodynamic effects when COMPOUND A was taken in the fasted state or when food was administered 60 minutes following COMPOUND A dosing (Table 15).

The pharmacodynamic effect of 900 mg once-daily and 450 mg twice-daily was examined as part of the multiple ascending dose phase of Study 1. A summary of the pharmacodynamic effect on receptor occupancy by dosing regimen is presented in Table 21.

TABLE 21
Summary of Receptor Occupancy Pharmacodynamics
Once Daily and Twice Daily (Mean ± SD).

| 900 mg QD (N = 7) | | 450 mg BID (N = 8) | |
|---|---|---|---|
| Day 1 | Day 5 | Day 1 | Day 5^{a} |

Receptor Occupancy

| | | | | |
|---|---|---|---|---|
| ROₘₐₓ (%) | 94.5 ± 2.61 | 94.9 ± 1.97 | 86.5 ± 4.11 | 91.9 ± 3.42 |
| Tₘₐₓ for RO (h) | 5.4 ± 3.9 | 3.3 ± 6.2 | 16 ± 6.2 | 2.3 ± 1.6 |
| Average RO (%) | 80.7 ± 6.7 | 79.2 ± 8.7 | 76.4 ± 8.4 | 85.3 ± 7.3 |

Receptor Expression

| | | | | |
|---|---|---|---|---|
| REₘₐₓ (%) | −47.3 ± 5.0 | −50.1 ± 5.3 | −42.6 ± 4.6 | −47.3 ± 4.3 |
| Tₘₐₓ for RE (h) | 13 ± 1.4 | 11 ± 3.9 | 19 ± 4.7 | 5.6 ± 5.1 |
| Average RE (%) | −38.3 ± 4.9 | −44.6 ± 6.3 | −33.4 ± 4.4 | −44.3 ± 4.2 |

^{a}N = 7

On Day 1, the 900 mg once-daily regimen had a mean peak receptor occupancy of 94.5% compared to 86.5% for the 450 mg twice-daily regimen. While both treatment regimens resulted in a similar peak receptor occupancy on Day 5 (94.9% and 91.9% for 900 mg QD and 450 mg BID, respectively), the twice daily regimen provided a more sustained pharmacodynamic effect. Notably, the AUEC on Day 5 was higher for the twice daily regimen compared to the once daily regimen. The average receptor occupancy based on the 24-hour area under the effect curve (AUEC) on Day 5 was 85.3% for the twice daily regimen and 79.2% for the once daily regimen. The BID regimen also provided a sustained effect as noted by the minimal difference in the peak and trough receptor occupancy. In addition, the intersubject variability in receptor occupancy at trough for the 450 mg BID treatment was 11.3%-15.2% on Day 5 compared to 26.3%-33.6% for the 900 mg QD treatment, suggesting a more consistent effect for the BID regimen.

The steady-state CD4⁺ α4β7 memory T cell percent receptor occupancy pharmacodynamics following twice daily COMPOUND A as the IR tablet or as the liquid solution is presented in FIG. 23B. The steady-state receptor occupancy pharmacodynamics is summarized in Table 22.

TABLE 22
Steady-State Pharmacodynamics of Compound A following
Oral Dosing of 450 mg Twice-Daily as a Liquid
Solution and as an IR Tablet (Mean ± SD)

| | Liquid Solution | IR Tablet 450 mg |
|---|---|---|
| | 450 mg BID (N = 9) | BID (N = 9) |
| ROₘₐₓ (%) | 93.8 ± 2.82 | 91.9 ± 4.56 |
| Tₘₐₓ for RO (h) | 4.0 (1.0, 8.0) | 4.0 (1.0, 8.0) |
| Average RO₀₋₂₄ (% · h) | 85.8 ± 4.3 | 83.6 ± 5.6 |
| Average RO₀₋₁₂ (% · h) | 89.7 ± 5.6 | 86.4 ± 5.2 |
| Average RO₁₂₋₂₄ (% · h) | 83.5 ± 6.6 | 80.8 ± 6.0 |

ROₘₐₓ maximum receptor occupancy, REₘₐₓ maximum receptor expression

Peak receptor occupancy was noted at 4 hours for both formulations. Mean steady-state peak receptor occupancy for the IR tablet was 91.9% with an average 24-hour receptor occupancy of 83.6% compared to a peak receptor occupancy of 93.8% and an average 24-hour receptor occupancy of 85.8% for the liquid solution.

### Pharmacokinetic-Pharmacodynamic Correlation

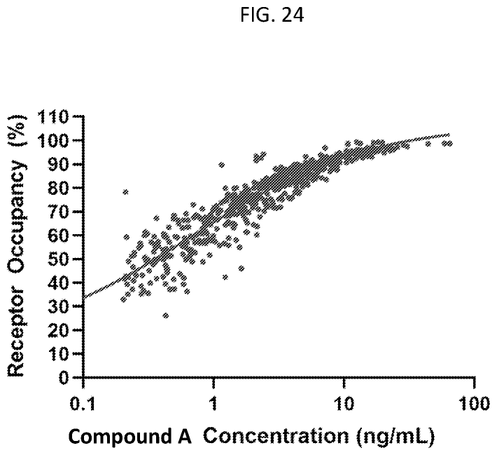

The in vivo COMPOUND A plasma concentration-receptor occupancy relationship was characterized using a sigmoid Emax (Hill) model (FIG. 24). The estimated IC₅₀ and IC₈₀ for receptor occupancy were 0.69 ng/mL and 5.9 ng/mL, respectively.

### Discussion

COMPOUND A is an oral intestinally restricted peptide that binds specifically to the α4β7 integrin on leukocytes that is being developed in a Phase 2 study as a potential oral therapy for patients with ulcerative colitis. The GI-restricted nature of the peptide and enhanced gastrointestinal stability allow local effect and has the potential to enhance efficacy while minimizing the potential for adverse events associated with systemic exposure.

The primary objective of these studies was to assess the safety/tolerability of COMPOUND A after single and multiple dosing. The secondary objectives were to evaluate the pharmacokinetic and pharmacodynamic profile of COMPOUND A after single and multiple ascending oral dose administration; to assess the effects of a food on the pharmacokinetics and pharmacokinetics; to compare once-daily and twice-a-day dosing; and to describe the pharmacokinetics and pharmacodynamics of an immediate-release formulation of COMPOUND A.

COMPOUND A was well tolerated following single doses of up to 1400 mg and multiple doses of up to 1400 mg once daily for 14 days in the first-in-human study. TEAEs were all mild except for 1 report of severe headache following a single administration of the lowest dose of COMPOUND A (100 mg) and a report of influenza reported following 900 mg once daily. None of the TEAEs led to subject withdrawal from the study. Treatment-emergent adverse events noted in two or more subject following repeated dosing included abdominal discomfort, flatulence, upper respiratory tract infection, back pain, dizziness, and headache with headaches being the most frequently reported TEAEs. Treatment with COMPOUND A did not result in any safety findings with regards to clinically meaningful changes in vital signs, clinical laboratory values and no evidence of QTc prolongation was observed. There was no difference in the treatment-emergent adverse event profile following dosing of COMPOUND A twice daily as an IR tablet or as a liquid solution.

Following single oral doses, COMPOUND A had a moderate rate of absorption with maximum plasma concentrations noted at approximately 4 hours. The increase in COMPOUND A AUC was approximately dose-proportional whereas the increase in Cₘₐₓ was slightly less than dose proportional. COMPOUND A demonstrated low systemic exposure following single and multiple dosing. The terminal half-life was 3.1 to 5.7 hours in the fasted state and 5.2 to 7.7 hours in the fed state. Consistent with the terminal half-life, when administered once daily and twice-daily, the accumulation of COMPOUND A was approximately 0.9 and 1.6-fold, respectively. There was an absence of time-dependent pharmacokinetics as evidenced by the similar AUC_{inf} Day 1 and AUCₜ on Day 14 (Supplementary Table 4).

There was a dose dependent increase in α4β7 receptor occupancy following COMPOUND A administration, reaching a mean peak receptor occupancy greater than 90% with doses of 900 mg. Trough receptor occupancy following once-daily dosing of 100 mg and 1000 mg COMPOUND A was approximately 25.4% and 78.6%, respectively, and 79.2% following 450 mg COMPOUND A twice daily. These receptor occupancy data indicate that COMPOUND A concentrations remain at a sufficient level to allow once- or twice-daily dosing. PK/PD correlation showed concentration-dependent receptor occupancy with an asymptote at complete receptor occupancy with an estimated IC₅₀ of 0.69 ng/mL and an IC₈₀ of 5.9 ng/mL. The estimated IC₅₀ for receptor occupancy noted in humans (0.69 ng/mL) compares very favorably with the potency of COMPOUND A against memory CD4+ T cells expressing α4β7 isolated from human peripheral blood mononuclear cells to recombinant MAdCAM1 (0.73 ng/mL).

Systemic concentrations of COMPOUND A following oral administration were generally low, consistent with the intestinally restricted nature of the drug and the very low oral bioavailability (<1%) that has been noted in mice and cynomolgus monkeys. There was a dose-dependent increase in fecal recovery of COMPOUND A following oral administrations, ranging from approximately 1-2% at 300 mg to 16.8% at 1000 mg. COMPOUND A is a small disulfide-containing cyclic peptide. Orally administered peptides encounter a harsh environment along the gastrointestinal tract, including pH conditions ranging from pH<2 stomach to pH 8 in the duodenum, as well as proteolytic enzymes such as gastric hydrolases (pepsins), pancreatic hydrolases (trypsin, chymotrypsin, elastase, aminopeptidases, and carboxypeptidase A and B), and intestinal brush-border membrane bound enzymes (carboxypeptidases, endopeptidases, and aminopeptidases).²⁹ The highly acidic environment in the stomach results in degradation of peptide drugs through destabilization of the three-dimensional structure. Peptide and protein stability in the gastrointestinal tract is an inherent problem associated with oral administration, whether for local action or for systemic delivery. Numerous studies have indicated that various factors such as amino acid sequence, molecular size, exposure of the gastrointestinal environment, including pH and enzymatic action play a key role in determining peptide stability and potential for oral absorption. Cyclization though sulfide bond linkage, and N-methylation provide some resistance to enzymatic degradation and may also improve oral absorption of Compound A.

The presence of low detectable intact concentrations in the plasma following oral administration indicates that COMPOUND A is able to traverse the gastrointestinal wall. In addition, approximately 0.03% to 0.06% of the drug was detected intact in the urine. Orally administered peptides typically have a low oral bioavailability. While the systemic concentrations of COMPOUND A are low, they were sufficient to achieve and maintain greater than 80% receptor occupancy at trough following once-daily or twice-daily dosing.

Administration of COMPOUND A within 30 minutes of a high fat meal reduced the oral absorption of COMPOUND A. While there was not a direct correlation between systemic exposure and fecal recovery, directionally, the data indicated that corresponding with the reduction in absorption following the high fat meal, there was an increase in fecal recovery

The steady-state pharmacokinetic and pharmacodynamic profile of the immediate-release tablet formulation of COMPOUND A was generally similar to the liquid formulation used in the first-in-human study. Twice-daily dosing of 450 mg COMPOUND A as IR tablets resulted in sustained pharmacokinetics and an average receptor occupancy of ˜84%.

### Conclusions

Compound A was dosed in 97 healthy male volunteers. In Part 1 single-ascending doses of Compound A, up to a maximum daily dose of 1400 mg, and the effect of food was studied; in Part 2 multiple ascending doses up to 1000 mg were tested as once daily dosing for up to 14 days. Additionally, 900 mg Compound A once daily for 5 days was compared to 450 mg Compound A twice daily for 5 days. The study drug was well-tolerated; there were no dose-limiting toxicities observed. With one exception, all adverse events were of mild to moderate severity. One serious adverse event of influenza characterized as severe was reported as being possibly related to study drug in a subject approximately 36 hours after receiving Compound A. The diagnosis was Influenza A confirmed by testing of flu swabs. The subject had an uneventful recovery.

The maximally tolerated dose for both single and multiple dosing was the highest dose tested, 1400 mg as a single dose and 1000 mg multiple dose. Minimal plasma exposure for both single and multiple dosing was observed confirming that the drug was largely GI-restricted. Dose-dependent increases in blood receptor occupancy and reduction in receptor expression were observed, thus supporting target engagement and pharmacologic activity of Compound A in healthy volunteers.

To support use of a tablet formulation, a multiple-dose crossover pharmacokinetic and pharmacodynamic study was conducted in 10 healthy subjects following dosing of 450 mg as an oral solution administered twice daily for 5 days or as immediate-release tablets administered twice daily for 5 days. On average, the IR tablet had a slightly lower peak Compound A plasma concentration and AUC values (˜15-18%) than the solution on Day 5, a difference that is not considered clinically meaningful. Mean steady-state peak receptor occupancy was >90% for both formulations and the average receptor occupancy based on the 24-hour area under the effect curve (AUEC) on Day 5 was comparable for the 2 formulations.

Following single and multiple ascending doses, COMPOUND A was safe and well tolerated when given orally to healthy subjects over a wide dose range. Consistent with a GI-restricted peptide, COMPOUND A had low systemic exposure with a pharmacokinetic profile that supports once or twice daily dosing. Twice daily dosing of COMPOUND A resulted in a sustained receptor occupancy. The safety, tolerability and PK/PD profile of COMPOUND A in healthy subjects supports the continued clinical evaluation of this novel gastrointestinal-restricted targeted treatment for inflammatory bowel diseases.

### Example 8

### Randomized, Double-Blind, Placebo-Controlled Study to Evaluate Safety and Efficacy of Oral Compound A in Subjects with Moderate to Severe Active Ulcerative Colitis

A phase 2 randomized, double-blind, placebo-controlled clinical study in human patients with moderated to severe ulcerative colitis is conducted to demonstrate safety, tolerability, and efficacy of treatment with oral Compound A. The study also evaluates the pharmacokinetic (PK) and pharmacodynamics (PD) and biomarker responses to treatment with oral Compound A.

### Study Design

This is a two-part study: Part 1 is a randomized, double-blind, placebo-controlled, parallel design 12-week induction treatment period in patients with moderate to severe active UC; and Part 2 is an extended treatment period of 40 weeks that will include subjects who successfully complete Part 1. Subjects who complete the Week 12 visit for Part 1 will be eligible to enter Part 2.

Part 1: Induction Treatment Period (ITP):

Part 1 is a 12-week randomized, double-blind, placebo-controlled, parallel design study in adult subjects with moderate to severe active UC. Eligible subjects are randomized 1:1:1 to Compound A 450 mg twice daily (BID), Compound A 150 mg BID, or placebo BID. Subjects must have a biopsy-confirmed diagnosis of UC. To satisfy inclusion criteria, eligible subjects must have had a prior inadequate initial response, loss of response or intolerance to an older conventional therapy for UC (i.e., a corticosteroid, aminosalicylate or immunomodulator) or prior inadequate initial response, loss of response or intolerance to a newer biologic therapy (i.e., a TNFα antagonist or an IL12/23 antagonist). Subjects with a history of prior vedolizumab treatment will be excluded. Randomization will be stratified by prior failure to a TNFα antagonist or an IL-12/23 antagonist.

Eligible subjects satisfy the following inclusion criteria:
Male and female subjects age 18 (or the minimum country specific age of consent if>18) to 75 years;
Subject understands the study procedures and agrees to participate in the study by giving written informed consent;
Diagnosis of UC supported by appropriate documentation of biopsy results consistent with UC
Moderate to severe active UC; and
Demonstrated inadequate response, loss of response, or intolerance of at least 1 of oral aminosalicylates (5-ASAs), corticosteroids, immunomodulators, or a biologic (excluding vedolizumab),
   and eligible subjects do not meet the following exclusion criteria:
Subject with a current diagnosis of Crohn's disease (CD), indeterminate colitis (IC), microscopic colitis, ischemic colitis, radiation colitis;
History of colonic dysplasia other than completely removed low-grade dysplastic lesion;
History of active bacterial, viral, fungal or mycobacterial infection requiring hospitalization or IV antibiotic/anti-infective treatment within 4 weeks of screening or oral antibiotics/anti-infectives within 2 weeks of screening;
Prior treatment with vedolizumab, natalizumab, or any agent targeting the α4β7 or β1 integrin or planned during the study;
Positive stool test for C. difficile;
Chronic recurrent or serious infection;
Known primary or secondary immunodeficiency;
Pregnant or lactating female or considering becoming pregnant during the study or within 30 days after the last dose of study medication; and
History of any major neurological disorders.

Eligible subjects are randomized 1:1:1 to Compound A 450 mg twice daily (BID), Compound A 450 150 mg BID, or placebo BID.

Part 2: Extended Treatment Period (ETP):

Subjects who complete the Week 12 visit for Part 1, including components of Adapted Mayo Score, will be eligible to enter Part 2. All Part 1 completers will be eligible to enter into Part 2, the extended treatment period, at the discretion of the investigator. Subjects will be assigned to the appropriate extended treatment arm in a blinded fashion. All subjects continuing into Part 2 will receive Compound A.

### Test Product(s), Dose and Mode of Administration:

Compound A (300 mg and 150 mg) and matching placebo tablets will be administered orally. Both Compound A strengths and placebo will have the same appearance.

### Outcome Analysis:

Primary outcome measures include the proportion of subjects achieving clinical remission at Week 12 compared to placebo. Clinical remission is determined using the Adapted Mayo score (sum of 3 subscores from the Mayo score):
Stool frequency subscore (SFS)
Rectal bleeding subscore (RBS)
Endoscopic subscore (ESS)

Secondary outcome measures include a comparison between Compound A high-dose and low-dose individually to placebo:
Proportion of subjects with endoscopic improvement.
Proportion of subjects achieving endoscopic remission.
Proportion of subjects with histological improvement.
Proportion of subjects achieving histological remission.
Proportion of subjects with mucosal healing.

Other outcome measures include the proportion of subjects achieving clinical remission at Week 52. Clinical remission is determined using the Adapted Mayo score (sum of 3 subscores from the Mayo score):
Stool frequency subscore (SFS)
Rectal bleeding subscore (RBS)
Endoscopic subscore (ESS).

### Assessment of Efficacy

Efficacy is assessed, at least in part, based on the Mayo score. The Mayo score includes 4 components: Stool Frequency Subscore (SFS), Rectal Bleeding Subscore (RBS), Endoscopic Subscore (ESS) and Physician's Global Assessment (PGA). Each of the individual scores range from 0 to 3 with higher number indicating higher severity):
Complete Mayo Score is a sum of all 4 subscores (SFS, RBS, ESS and PGA) and ranges from 0 to 12 points.
Adapted Mayo Score is a sum of 3 subscores (SFS, RBS and ESS). The Adapted Mayo score ranges from 0 to 9 points.
Partial Mayo Score is a sum of 3 subscores (SFS, RBS and PGA) ranging from 0 to 9 points.
Endoscopic subscore (ESS)≥1 (modified so that a score of 1 does not include friability).

### Results

It is expected that the treatment with any of the dosages of Compound A will be safe, and that treatment with either 450 mg BID or 150 mg BID will show statistically significant improvement in Complete Mayo Score, Adapted Mayo Score, and/or Partial Mayo Score as compared to treatment with placebo, thus demonstrating the effectiveness of these dosages of Compound A for treating ulcerative colitis.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

What is claimed is:

1. A method of treating an inflammatory bowel disease (IBD) in a subject in need thereof, comprising administering to the subject an α4β7 integrin antagonist, wherein the antagonist is administered to the patient orally at a dose of about 100 mg to about 500 mg, once or twice daily, wherein the antagonist is a peptide dimer compound comprising two peptides, or a pharmaceutically acceptable salt thereof; wherein each of the two peptides comprises or consists of any of the sequences:
(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH;
(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;
(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;
(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;
(SEQ ID NO: 5)
2-methylbenzoyl-(N-Me-Arg)-Ser-Asp-Thr-Leu-Pen-
Phe(4-tBu)-(β-homo-Glu)-(D-Lys)-OH;
(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂;
(SEQ ID NO: 6)
Pen-(N-Me-Arg)-Ser-Asp-Thr-Leu-Pen-Phe(4-tBu)-
(β-homo-Glu)-(D-Lys)-OH;
(SEQ ID NO: 6)
Pen-(N-Me-Arg)-Ser-Asp-Thr-Leu-Pen-Phe(4-tBu)-
(β-homo-Glu)-(D-Lys)-NH2;
(SEQ ID NO: 7)
Pen-(N-Me-Arg)-Ser-Asp-Thr-Leu-Pen-Phe(4-tBu)-
(β-homo-Glu)-Pro-(D-Lys)-OH;
(SEQ ID NO: 7)
Pen-(N-Me-Arg)-Ser-Asp-Thr-Leu-Pen-Phe(4-tBu)-
(β-homo-Glu)-Pro-(D-Lys)-NH2;
(SEQ ID NO: 8)
Pen-(N-Me-Arg)-Ser-Asp-Thr-Leu-Pen-Phe(4-tBu)-
(β-homo-Glu)-(D-Pro)-(D-Lys)-OH;
or
(SEQ ID NO: 8)
Pen-(N-Me-Arg)-Ser-Asp-Thr-Leu-Pen-Phe(4-tBu)-
(β-homo-Glu)-(D-Pro)-(D-Lys)-NH2;
wherein each of the two peptides comprises a thioether bond between the 2-methylbenzoyl and the Pen or a disulfide bond between the two Pens, wherein the two peptides are linked by a linker moiety bound to the D-Lys amino acids of the two peptides, and wherein the linker moiety is diglycolic acid (DIG).

2. The method of claim 1, wherein each of the two peptides consists of the sequence:
(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

3. The method of claim 1, wherein each of the two peptides consists of the sequence:
(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

4. The method of claim 1, wherein each of the two peptides consists of the sequence:
(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

5. The method of claim 1, wherein each of the two peptides consists of the sequence:
(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

6. The method of claim 1, wherein each of the two peptides consists of the sequence:
(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

7. The method of claim 1, wherein each of the two peptides consists of the sequence:
(SEQ ID NO: 5)
2-methylbenzoyl-(N-Me-Arg)-Ser-Asp-Thr-Leu-Pen-
Phe(4-tBu)-(β-homo-Glu)-(D-Lys)-OH,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

8. The method of claim 1, wherein the peptide dimer compound or pharmaceutically acceptable salt thereof is: 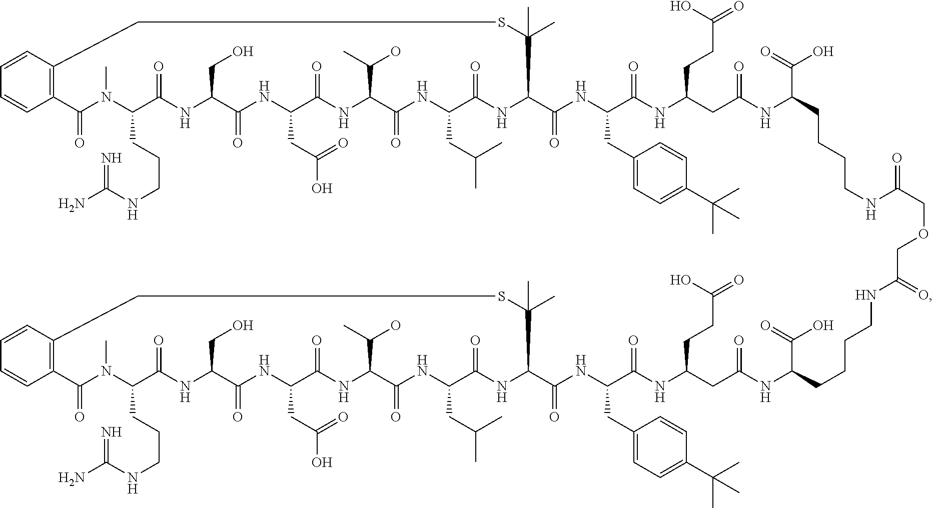 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]
or a pharmaceutically acceptable salt thereof.

9. The method of claim 1, wherein the peptide dimer compound or pharmaceutically acceptable salt thereof is: 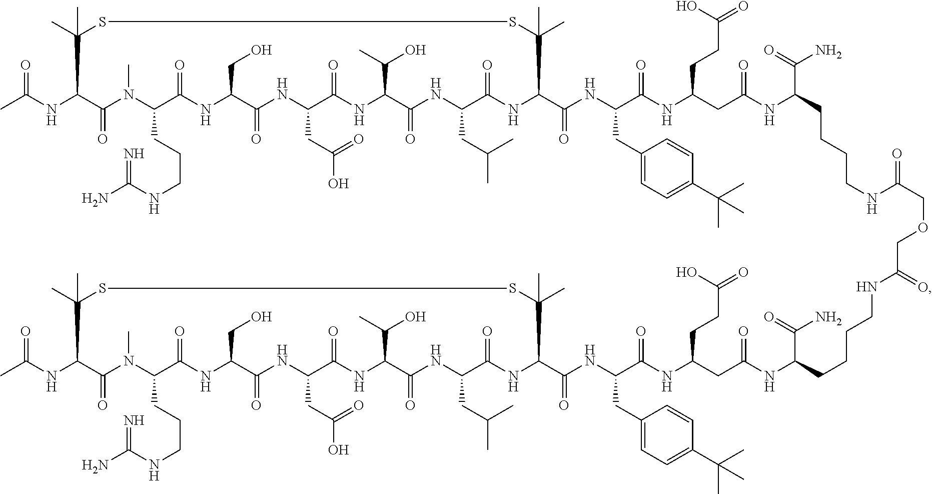 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)[C@@H](NC(C)=O)C(C)(C)SSC2(C)C)C(N)=O)C(N)=O)C(C)(C)SSC1(C)C]
or a pharmaceutically acceptable salt thereof.

10. The method of claim 1, wherein each of the two peptides consists of the sequence:
(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

11. The method of claim 1, wherein each of the two peptides consists of the sequence:
(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

20. The method of claim 9, wherein each of the two peptides consists of the sequence:
(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

12. The method of claim 1, wherein each of the two peptides consists of the sequence:
(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

13. The method of claim 1, wherein each of the two peptides consists of the sequence:
(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

14. The method of claim 1, wherein each of the two peptides consists of the sequence:
(SEQ ID NO: 5)
2-methylbenzoyl-(N-Me-Arg)-Ser-Asp-Thr-Leu-Pen-
Phe(4-tBu)-(β-homo-Glu)-(D-Lys)-OH,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

15. The method of claim 1, wherein the peptide dimer compound or pharmaceutically acceptable salt thereof is: 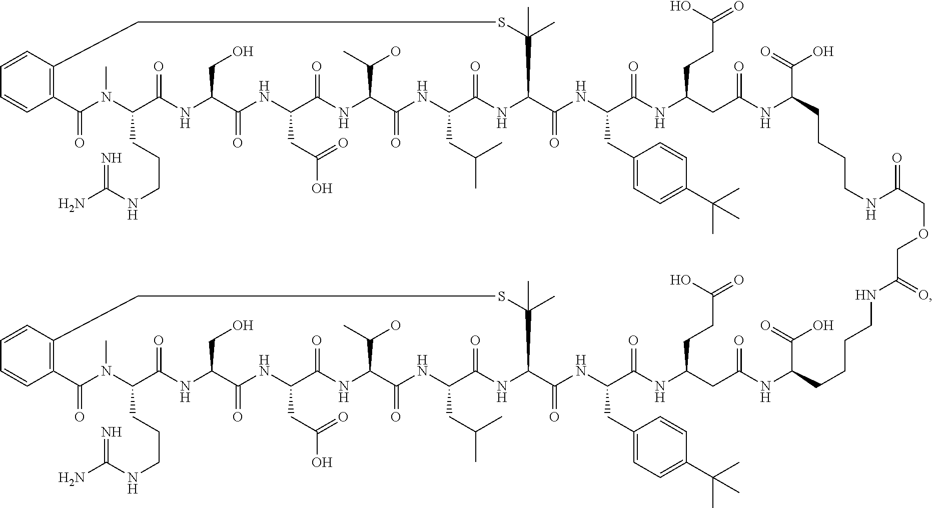 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]
or a pharmaceutically acceptable salt thereof.

16. The method of claim 1, wherein the peptide dimer compound or pharmaceutically acceptable salt thereof is: 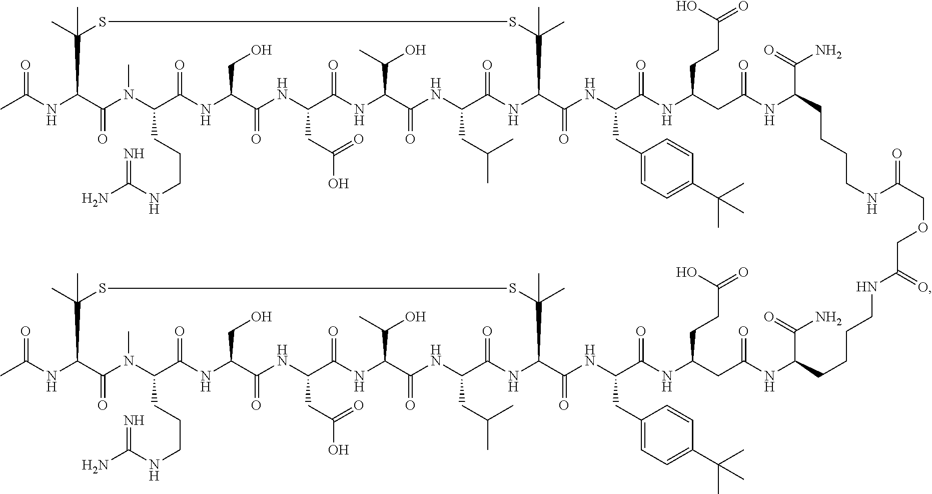 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)[C@@H](NC(C)=O)C(C)(C)SSC2(C)C)C(N)=O)C(N)=O)C(C)(C)SSC1(C)C]
or a pharmaceutically acceptable salt thereof.

17. The method according to any one of claims 1-16, wherein the peptide dimer compound or pharmaceutically acceptable salt thereof is administered to the subject at a dose of about 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, or 500.0 mg.

18. The method according to claim 17, wherein the peptide dimer compound or pharmaceutically acceptable salt thereof is administered to the subject at a dose of about 150 mg.

19. The method according to claim 17, wherein the peptide dimer compound or pharmaceutically acceptable salt thereof is administered to the subject at a dose of about 450 mg.

20. The method according to claim 18 or claim 19, wherein the dose is administered to the subject twice daily.

21. The method according to any one of claims 1-20, wherein the pharmaceutically acceptable salt of the peptide dimer compound is an acetate salt.

22. The method according to any one of claims 1-21, wherein the dosage administered results in a non-saturating blood receptor occupancy (% RO), optionally when measured at peak blood or serum levels of the antagonist.

23. The method according to claim 22, wherein the dosage administered results in less than 90% RO, less than 80% RO, less than 70% RO, less than 60% RO, or less than 50% RO, optionally when measured at peak blood or serum levels of the antagonist.

24. The method according to any one of claims 1-21, wherein the method inhibits MadCAM1-mediated T cell proliferation in the gastrointestinal tract.

25. The method according to any one of claims 1-21, wherein the method reduces cell surface expression of β7 on CD4+ T cells in the gastrointestinal tract.

26. The method according to any one of claims 1-21, wherein the method:
i) induces internalization of α4β7 integrin on CD4+ T memory cells;
ii) causes reduced adhesion of CD4+ T memory cells to MAdCAM1 in the gastrointestinal tract; and/or
iii) inhibits homing of T cells to the gastrointestinal tract, optionally to the ileal lamina propia, Peyer's Patches, mesenteric lymph nodes, small intestine, and/or colon.

27. The method of any one of claims 1-26, wherein the IBD is ulcerative colitis.

28. The method of any one of claims 1-26, wherein the IBD is Crohn's disease.

29. The method of any one of claims, wherein the method results in one or more of the following pharmacokinetic parameters in plasma of the subject:
Cmax (ng/mL) of 1-25;
Tmax (h) of 1-5;
AUCₜ (ng·h/mL) of 10-250 AUC_{inf} (ng·h/mL) of 10-300;
t_{1/2} (h) of 3-10;
AUCₜₐᵤ (ng·h/mL) of 30-130;
Ctrough (ng/mL) of 1-5;
accumulation Cmax (ng·mL) of 0.5-2.5; and
accumulation AUCₜ (ng·h/mL) of 0.5-3.0.

30. The method of any one of claims, wherein the method results in one or more of the following pharmacodynamic parameters in plasma of the subject:
ROmax (%) of 50-100;
change in receptor expressionₘₐₓ (%) of −20 to −60;
average change in receptor expression (%) of −10 to −55;
steady state ROmax (%) of 80-100;
average RO₀₋₂₄ (% h) of 50-95;
average RO₀₋₁₂ (% h) of 80-95; and
average RO₁₂₋₂₄ (% h) of 70-90.

31. A method of treating an inflammatory disease or disorder in a subject in need thereof, comprising administering to the subject an α4β7 integrin antagonist, wherein the antagonist is administered at a dosage that results in a non-saturating blood receptor occupancy (% RO), optionally when measured at peak blood or serum levels of the antagonist.

32. The method of claim 11, wherein the antagonist is administered at a dosage that results in less than 90% blood RO, less than 80% blood RO, less than 70% blood RO, less than 60% blood RO, or less than 50% blood RO, optionally when measured at peak blood or serum levels of the antagonist.

33. The method of claim 31 or claim 32, wherein the antagonist is present in a pharmaceutical composition formulated for a route of administration selected from oral administration, parenteral administration, subcutaneous administration, buccal administration, nasal administration, administration by inhalation, topical administration, and rectal administration.

34. The method of any one of claims 31-33, wherein the antagonist is administered orally or rectally.

35. The method of any one of claims 31-34, wherein the disease or disorder is selected from the group consisting of: Inflammatory Bowel Disease (IBD), adult IBD, pediatric IBD, adolescent IBD, ulcerative colitis, Crohn's disease, Celiac disease (nontropicalSprue), enteropathy associated with seronegative arthropathies, microscopic colitis, collagenous colitis, eosinophilic gastroenteritis, radiotherapy, chemotherapy, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, primary sclerosing cholangitis, human immunodeficiency virus (HIV) infection in the GI tract, eosinophilic asthma, eosinophilic esophagitis, gastritis, colitis, microscopic colitis, and graft versus host disease (GVDH).

36. The method of any one of claims 31-35, wherein the disease or disorder is an IBD.

37. The method of claim 36, wherein the IBD is ulcerative colitis.

38. The method of claim 36, wherein the IBD is Crohn's disease.

39. The method of any one of claims 31-38, wherein the antagonist is a peptide dimer compound comprising two peptides, or a pharmaceutically acceptable salt thereof; wherein each of the two peptides comprises or consists of any of the sequences:
(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH;
(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;
(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;
(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;
(SEQ ID NO: 5)
2-methylbenzoyl-(N-Me-Arg)-Ser-Asp-Thr-Leu-Pen-
Phe(4-tBu)-(β-homo-Glu)-(D-Lys)-OH;
(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂;
(SEQ ID NO: 6)
Pen-(N-Me-Arg)-Ser-Asp-Thr-Leu-Pen-Phe(4-tBu)-
(β-homo-Glu)-(D-Lys)-OH;
(SEQ ID NO: 6)
Pen-(N-Me-Arg)-Ser-Asp-Thr-Leu-Pen-Phe(4-tBu)-
(β-homo-Glu)-(D-Lys)-NH2;
(SEQ ID NO: 7)
Pen-(N-Me-Arg)-Ser-Asp-Thr-Leu-Pen-Phe(4-tBu)-
(β-homo-Glu)-Pro-(D-Lys)-OH;
(SEQ ID NO: 7)
Pen-(N-Me-Arg)-Ser-Asp-Thr-Leu-Pen-Phe(4-tBu)-
(β-homo-Glu)-Pro-(D-Lys)-NH2;
(SEQ ID NO: 8)
Pen-(N-Me-Arg)-Ser-Asp-Thr-Leu-Pen-Phe(4-tBu)-
(β-homo-Glu)-(D-Pro)-(D-Lys)-OH;
or
(SEQ ID NO: 8)
Pen-(N-Me-Arg)-Ser-Asp-Thr-Leu-Pen-Phe(4-tBu)-
(β-homo-Glu)-(D-Pro)-(D-Lys)-NH2;
wherein each of the two peptides comprises a thioether bond between the 2-methylbenzoyl and the Pen; or a disulfide between the two Pens; wherein the two peptides are linked by a linker moiety bound to the D-Lys amino acids of the two peptides, and wherein the linker moiety is diglycolic acid (DIG).

40. The method of claim 39, wherein each of the two peptides consists of the sequence:
(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

41. The method of claim 39, wherein each of the two peptides consists of the sequence:
(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

42. The method of claim 39, wherein each of the two peptides consists of the sequence:
(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

43. The method of claim 39, wherein each of the two peptides consists of the sequence:
(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

44. The method of claim 39, wherein each of the two peptides consists of the sequence:
(SEQ ID NO: 5)
2-methylbenzoyl-(N-Me-Arg)-Ser-Asp-Thr-Leu-Pen-
Phe(4-tBu)-(β-homo-Glu)-(D-Lys)-NH₂,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

45. The method of claim 39, wherein each of the two peptides consists of the sequence:
(SEQ ID NO: 5)
2-methylbenzoyl-(N-Me-Arg)-Ser-Asp-Thr-Leu-Pen-
Phe(4-tBu)-(β-homo-Glu)-(D-Lys)-OH,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

46. The method of claim 39, wherein the peptide dimer compound or pharmaceutically acceptable salt thereof is: 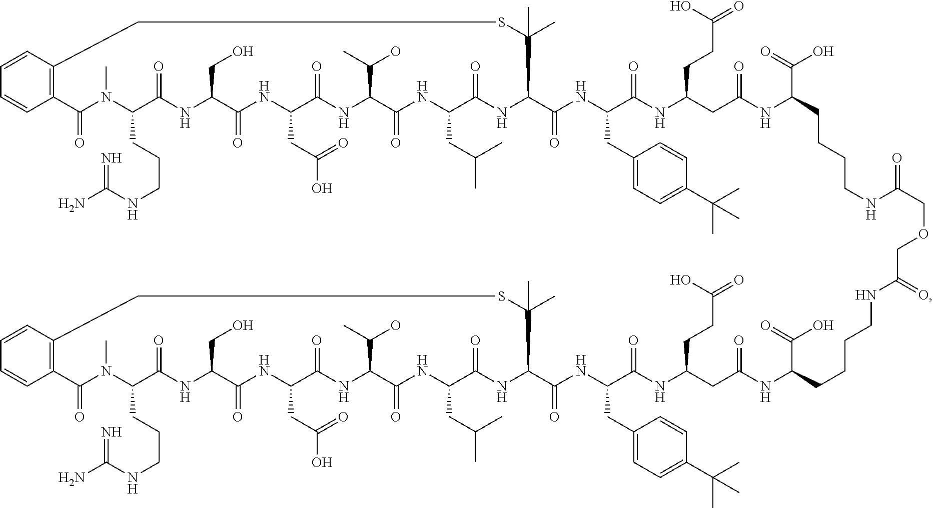 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]
or a pharmaceutically acceptable salt thereof.

47. The method of claim 39, wherein the peptide dimer compound or pharmaceutically acceptable salt thereof is: 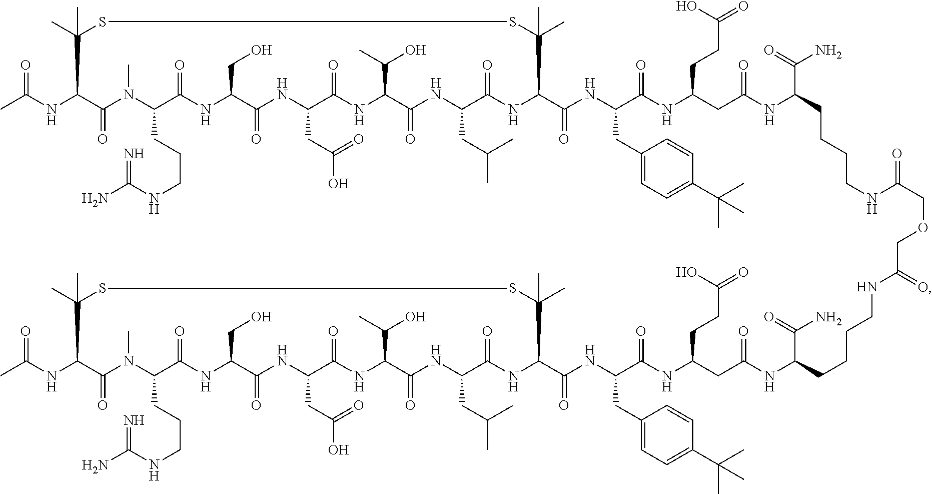 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)[C@@H](NC(C)=O)C(C)(C)SSC2(C)C)C(N)=O)C(N)=O)C(C)(C)SSC1(C)C]
or a pharmaceutically acceptable salt thereof.

48. The method of claim 39, wherein each of the two peptides consists of the sequence:
(SEQ ID NO: 1)
2-methylbenzoyl-(N-Me-Arg)-Ser-Asp-Thr-Leu-Pen-
Phe(4-tBu)-(β-homo-Glu)-(D-Glu)-(D-Lys)-OH,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

49. The method of claim 39, wherein each of the two peptides consists of the sequence:
(SEQ ID NO: 2)
2-methylbenzoyl-(N-Me-Arg)-Ser-Asp-Thr-Leu-Pen-
Phe(4-tBu)-(β-homo-Glu)-Gly-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

50. The method of claim 39, wherein each of the two peptides consists of the sequence:
(SEQ ID NO: 3)
2-methylbenzoyl-(N-Me-Arg)-Ser-Asp-Thr-Leu-Pen-
Phe(4-tBu)-(β-homo-Glu)-Pro-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

51. The method of claim 39, wherein each of the two peptides consists of the sequence:
(SEQ ID NO: 4)
2-methylbenzoyl-(N-Me-Arg)-Ser-Asp-Thr-Leu-Pen-
Phe(4-tBu)-(β-homo-Glu)-(D-Pro)-(D-Lys)-OH;
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

52. The method of claim 39, wherein each of the two peptides consists of the sequence:
(SEQ ID NO: 5)
2-methylbenzoyl-(N-Me-Arg)-Ser-Asp-Thr-Leu-Pen-
Phe(4-tBu)-(β-homo-Glu)-(D-Lys)-NH2,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

53. The method of claim 39, wherein each of the two peptides consists of the sequence:
(SEQ ID NO: 5)
2-methylbenzoyl-(N-Me-Arg)-Ser-Asp-Thr-Leu-Pen-
Phe(4-tBu)-(β-homo-Glu)-(D-Lys)-OH,
wherein each of the two peptides comprises a thioether bond between the 2-methylbenzoyl and the Pen, wherein the two peptides are linked by a linker moiety bound to the D-Lys amino acids of the two peptides, and wherein the linker moiety is diglycolic acid (DIG).

54. The method of claim 39, wherein the peptide dimer compound or pharmaceutically acceptable salt thereof is: 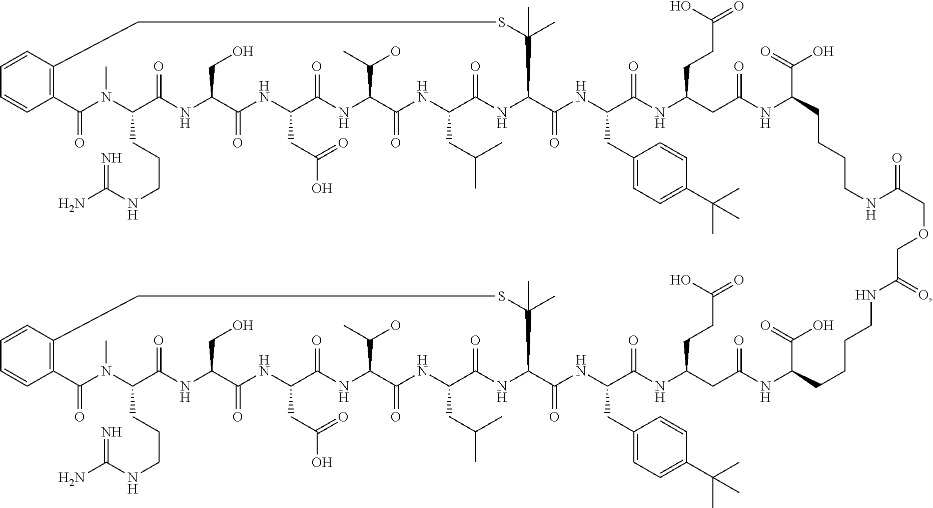 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]
or a pharmaceutically acceptable salt thereof.

55. The method of claim 39, wherein the peptide dimer compound or pharmaceutically acceptable salt thereof is: 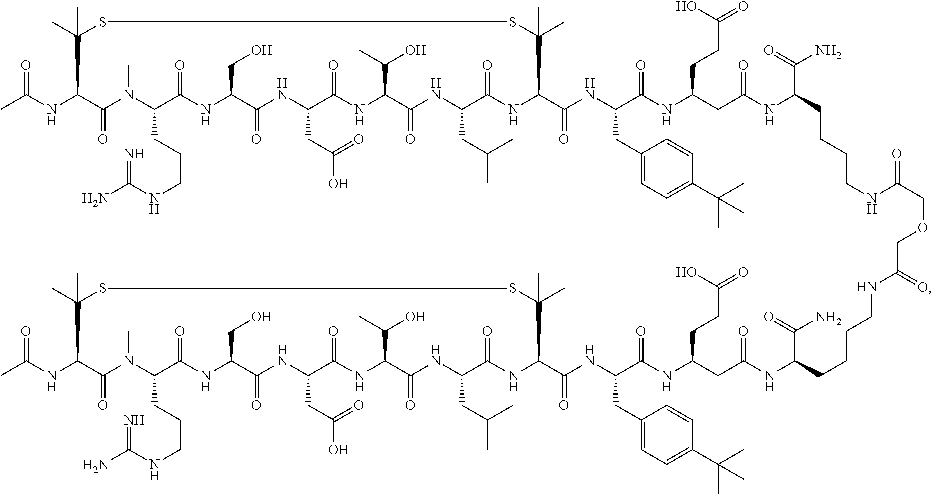 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)[C@@H](NC(C)=O)C(C)(C)SSC2(C)C)C(N)=O)C(N)=O)C(C)(C)SSC1(C)C]
or a pharmaceutically acceptable salt thereof.

56. The method according to any one of claims 39-55, wherein the peptide dimer compound or pharmaceutically acceptable salt thereof is administered to the subject at a dose of about 5, 6, 7, 8, 9, 10, 12.5, 25.0, 37.5, 50.0, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, or 500.0 mg.

57. The method of claim 56, wherein the dose is administered to the subject once a day or twice a day.

58. The method according to any one of claims 39-57, wherein the pharmaceutically acceptable salt of the peptide dimer compound is an acetate salt.

59. A pharmaceutical composition comprising a peptide dimer compound or pharmaceutically acceptable salt thereof disclosed in any one of claims 39-58.

60. The pharmaceutical composition of claim 59, wherein the composition is formulated for oral delivery, optionally wherein the composition comprises an enteric coating.

61. The method of any one of claims 39-58, wherein the method comprises administering to the subject the pharmaceutical composition of any one of claims 32-34.

62. The method of any one of claims 31-58 or 61, wherein the antagonist or pharmaceutically acceptable salt thereof inhibits binding of α4β7 integrin to MAdCAM1.

63. The method of any one of claims 31-58 or 61-62, wherein the antagonist or pharmaceutically acceptable salt thereof or the pharmaceutical composition is provided to the subject in need thereof at an interval sufficient to improve or ameliorate the condition.

64. The method of claim 63, wherein the interval is selected from the group consisting of: around the clock, hourly, every four hours, once daily, twice daily, three times daily, four times daily, every other day, weekly, bi-weekly, and monthly.

65. The method of claim 63 or claim 64, wherein the antagonist or pharmaceutically acceptable salt thereof or pharmaceutical composition is provided as an initial does followed by one or more subsequent doses, and the minimum interval between any two doses is a period of less than 1 day, and wherein each of the doses comprises an effective amount of the antagonist.

66. The method of claim 65, wherein the effective amount of the antagonist or pharmaceutically acceptable salt thereof or the pharmaceutical composition is sufficient to achieve at least one of the following:
a) about 50% or greater saturation of MAdCAM1 binding sites on α4β7 integrin molecules;
b) about 50% or greater inhibition of α4β7 integrin expression on the cell surface; and
c) about 50% or greater saturation of MAdCAM1 binding sites on α4β7 molecules and about 50% or greater inhibition of α4β7 integrin expression on the cell surface, wherein i) the saturation is maintained for a period consistent with a dosing frequency of no more than twice daily; ii) the inhibition is maintained for a period consistent with a dosing frequency of no more than twice daily; or iii) the saturation and the inhibition are each maintained for a period consistent with a dosing frequency of no more than twice daily.


SEQUENCE LISTING (8 sequences)

SEQ ID NO: 1 - protein, 10 aa, Artificial Sequence
  Made in lab - peptide antagonist
  MOD_RES (1)..(1): Xaa is N-Methyl-Arginine
  MOD_RES (1)..(1): N-Methyl-Arginine modified with 2-methylbenzoyl
  MOD_RES (6)..(6): Xaa is penicillamine
  MOD_RES (7)..(7): Xaa is (S)-2-amino-3-(4-tert-butyl- phenyl)propionic acid
  MOD_RES (8)..(8): Xaa is beta-homoglutamic acid
  MOD_RES (9)..(9): D form Glu
  MOD_RES (10)..(10): D form Lys
XSDTLXXXEK

SEQ ID NO: 2 - protein, 10 aa, Artificial Sequence
  Made in lab - peptide antagonist
  MOD_RES (1)..(1): Xaa is N-Methyl-Arginine
  MOD_RES (1)..(1): N-Methyl-Arginine modified with 2-methylbenzoyl
  MOD_RES (6)..(6): Xaa is penicillamine
  MOD_RES (7)..(7): Xaa is (S)-2-amino-3-(4-tert-butyl- phenyl)propionic acid
  MOD_RES (8)..(8): Xaa is beta-homoglutamic acid
  MOD_RES (10)..(10): D form Lys
XSDTLXXXGK

SEQ ID NO: 3 - protein, 10 aa, Artificial Sequence
  Made in lab - peptide antagonist
  MOD_RES (1)..(1): Xaa is N-Methyl-Arginine
  MOD_RES (1)..(1): N-Methyl-Arginine modified with 2-methylbenzoyl
  MOD_RES (6)..(6): Xaa is penicillamine
  MOD_RES (7)..(7): Xaa is (S)-2-amino-3-(4-tert-butyl- phenyl)propionic acid
  MOD_RES (8)..(8): Xaa is beta-homoglutamic acid
  MOD_RES (10)..(10): D form Lys
XSDTLXXXPK

SEQ ID NO: 4 - protein, 10 aa, Artificial Sequence
  Made in lab - peptide antagonist
  MOD_RES (1)..(1): Xaa is N-Methyl-Arginine
  MOD_RES (1)..(1): N-Methyl-Arginine modified with 2-methylbenzoyl
  MOD_RES (6)..(6): Xaa is penicillamine
  MOD_RES (7)..(7): Xaa is (S)-2-amino-3-(4-tert-butyl- phenyl)propionic acid
  MOD_RES (8)..(8): Xaa is beta-homoglutamic acid
  MOD_RES (9)..(9): D form Pro
  MOD_RES (10)..(10): D form Lys
XSDTLXXXPK

SEQ ID NO: 5 - protein, 9 aa, Artificial Sequence
  Made in lab - peptide antagonist
  MOD_RES (1)..(1): Xaa is N-Methyl-Arginine
  MOD_RES (1)..(1): N-Methyl-Arginine modified with 2-methylbenzoyl
  MOD_RES (6)..(6): Xaa is penicillamine
  MOD_RES (7)..(7): Xaa is (S)-2-amino-3-(4-tert-butyl- phenyl)propionic acid
  MOD_RES (8)..(8): Xaa is beta-homoglutamic acid
  MOD_RES (9)..(9): D form Lys
XSDTLXXXK

SEQ ID NO: 6 - protein, 10 aa, Artificial Sequence
  Made in lab - peptide antagonist
  MOD_RES (1)..(1): Xaa is Penicillamine
  MOD_RES (2)..(2): Xaa is N-Methyl-Arginine
  MOD_RES (7)..(7): Xaa is penicillamine
  MOD_RES (8)..(8): Xaa is (S)-2-amino-3-(4-tert-butyl- phenyl)propionic acid
  MOD_RES (9)..(9): Xaa is beta-homoglutamic acid
  MOD_RES (10)..(10): D form Lys
XXSDTLXXXK

SEQ ID NO: 7 - protein, 11 aa, Artificial Sequence
  Made in lab - peptide antagonist
  MOD_RES (1)..(1): Xaa is Penicillamine
  MOD_RES (2)..(2): Xaa is N-Methyl-Arginine
  MOD_RES (7)..(7): Xaa is penicillamine
  MOD_RES (8)..(8): Xaa is (S)-2-amino-3-(4-tert-butyl- phenyl)propionic acid
  MOD_RES (9)..(9): Xaa is beta-homoglutamic acid
  MOD_RES (11)..(11): D form Lys
XXSDTLXXXPK

SEQ ID NO: 8 - protein, 11 aa, Artificial Sequence
  Made in lab - peptide antagonist
  MOD_RES (1)..(1): Xaa is Penicillamine
  MOD_RES (2)..(2): Xaa is N-Methyl-Arginine
  MOD_RES (7)..(7): Xaa is penicillamine
  MOD_RES (8)..(8): Xaa is (S)-2-amino-3-(4-tert-butyl- phenyl)propionic acid
  MOD_RES (9)..(9): Xaa is beta-homoglutamic acid
  MOD_RES (10)..(10): D form Pro
  MOD_RES (11)..(11): D form Lys
XXSDTLXXXPK